(19) United States
(12) Patent Application Publication
(10) Pub. No.: US 2024/0287063 A1
(43) Pub. Date: Aug. 29, 2024

(54) METHIONINE ADENOSYLTRANSFERASE 2A INHIBITOR

(71) Applicant: NANJING CHIA TAI TIANQING PHARMACEUTICAL CO., LTD., Nanjing (CN)

(72) Inventors: Changyou MA, Nanjing (CN); Dandan HUANG, Nanjing (CN); Jincai SU, Nanjing (CN); Li MA, Nanjing (CN); Yebin Wu, Nanjing (CN); Qingyu DAI, Nanjing (CN); Huan ZHANG, Nanjing (CN); Rui Zuo, Nanjing (CN); Qiuhua ZHOU, Nanjing (CN); Jian WU, Nanjing (CN); Lei MIAO, Nanjing (CN); Dan XU, Nanjing (CN); Chunxia ZHU, Nanjing (CN); Zhoushan TIAN, Nanjing (CN)

(73) Assignee: NANJING CHIA TAI TIANQING PHARMACEUTICAL CO., LTD., Nanjing (CN)

(21) Appl. No.: 18/566,584

(22) PCT Filed: May 31, 2022
(86) PCT No.: PCT/CN2022/096370

§ 371 Date: Dec. 1, 2023

(30) Foreign Application Priority Data
Jun. 2, 2021 (CN) 20210612811.7

(51) Int. Cl. C07D 471/04; A61K 31/4375; A61K 31/519; C07D 455/02; C07D 519/00

(52) U.S. Cl. CPC C07D 471/04; A61K 31/4375; A61K 31/519; C07D 455/02; C07D 519/00

(57) ABSTRACT

The present invention provides a methionine adenosyltransferase 2A inhibitor. The structure of the methionine adenosyltransferase 2A inhibitor is as represented by general formula (I), and the definitions of substituents are as described in the specification. The present invention further provides a preparation method therefor. A compound represented by formula I provided by the present invention has significant methionine adenosyltransferase 2A inhibitory activity, and can be used in the treatment of diseases mediated by overexpression of methionine adenosyltransferase 2A.

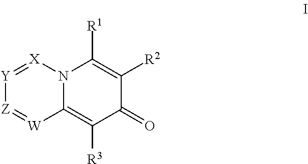

### TECHNICAL FIELD

The present invention relates to a methionine adenosyltransferase 2A inhibitor, which can be used for treating some types of cancers.

### BACKGROUND

Methionine adenosyltransferase (MAT) (also known as S-adenosylmethionine synthetase) is a cellular enzyme that catalyzes methionine and ATP to synthesize S-adenosylmethionine (SAM or AdoMet), and is considered a rate limiting step in a methionine cycle. SAM is a propylamino donor in polyamine biosynthesis and a main methyl donor used for DNA methylation, and is involved in gene transcription, cell proliferation, and generation of secondary metabolites.

Methionine adenosyltransferase 2A (MAT2A) is an enzyme that employs methionine (Met) and adenosine triphosphate (ATP) to generate s-adenosylmethionine (SAM). SAM is a main methyl donor in cells and is used for methylation of many substrates including DNA, RNA, and proteins. MTAP (methylthioadenosine phosphorylase) is an enzyme widely expressed in normal tissues, which catalyzes conversions of methylthioadenosine (MTA) to adenine and 5-methylthioribose-1-phosphate, adenine to adenosine monophosphate, and 5-methylthioribose-1-phosphate to methionine and formate. When purine synthesis is blocked, such as by antimetabolites, MTA can be used as an alternative purine source.

A gene encoding MTAP is located on a site of chromosome 9 and is often deleted in cancer patients from cells in the central nervous system, pancreas, esophagus, bladder, and lungs. Compared to cells expressing MTAP, the loss of MTAP leads to accumulation of MTA, making MTAP deficient cells more dependent on the generation of SAM and therefore more dependent on the activity of MAT2A. In screening of approximately 400 cancer cell lines, MAT2A knockdown resulted in a greater percentage loss of vitality in cells lacking MTAP compared with cells that normally expressed MTAP. Moreover, inducible knockdown of MAT2A protein reduced the growth of tumor in vivo. These results suggest that MAT2A inhibitors may provide a novel treatment approach for patients with tumors including those with MTAP deletion.

At present, Chinese patent application CN109890822A discloses a pyrazolopyrimidinone MAT2A inhibitor, WO2020123395A1 discloses a 2-oxoquinazoline derivative as a MAT2A inhibitor, and the present invention provides a new MAT2A inhibitor.

### SUMMARY OF THE INVENTION

In one aspect, the present invention provides a compound of formula I or a pharmaceutically acceptable salt thereof:

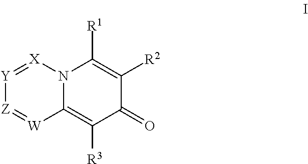

wherein X is selected from CR⁴ or N; Y is selected from CR⁵ or N; Z is selected from CR⁶ or N; W is selected from CR⁷ or N.
wherein R¹, R⁴, R⁵, R⁶, and R⁷ are independently selected from hydrogen, cyano, C2-C6 alkynyl, C8-C10 cycloalkynyl, halogen, hydroxyl, NH₂, (C1-C6 alkyl)-NR⁸—, (C1-C6 alkyl)-O—, (C1-C6 alkyl)-S—, C1-C6 alkyl, C3-C6 cycloalkyl, 6-10-membered aryl, C2-C6 alkenyl, or C3-C6 cycloalkenyl; the C1-C6 alkyl, (C1-C6 alkyl)-NR⁸—, (C1-C6 alkyl)-O—, C2-C6 alkenyl, C3-C6 cycloalkyl, or C3-C6 cycloalkenyl is optionally substituted by halogen, cyano, hydroxyl, —NR⁸R⁹, C1-C3 alkyl, C1-C3 alkoxy, C2-C6 alkenyl, or C2-C6 alkynyl; the 6-10-membered aryl is optionally substituted by halogen, hydroxyl, cyano, —NR⁸R⁹, NO₂, C1-C3 alkyl, C1-C3 alkoxy, C2-C6 alkenyl, or C2-C6 alkynyl; or the 6-10-membered aryl is optionally substituted by halogen, hydroxyl, cyano, —NR⁸R⁹, or NO₂ substituted C1-C3 alkyl, C1-C3 alkoxy, C2-C6 alkenyl, or C2-C6 alkynyl.

In some embodiments, R¹, R⁴, R⁵, R⁶, and R⁷ are independently selected from hydrogen, cyano, C2-C6 alkynyl, halogen, hydroxyl, NH₂, (C1-C6 alkyl)-NR⁸—, (C1-C6 alkyl)-O—, (C1-C6 alkyl)-S—, C1-C6 alkyl, C3-C6 cycloalkyl, 6-10-membered aryl, C2-C6 alkenyl, or C3-C6 cycloalkenyl; the C1-C6 alkyl, C2-C6 alkenyl, C3-C6 cycloalkyl, or C3-C6 cycloalkenyl is optionally substituted by halogen, cyano, hydroxyl, —NR⁸R⁹, C1-C3 alkyl, C1-C3 alkoxy, C2-C6 alkenyl, or C2-C6 alkynyl; the 6-10-membered aryl is optionally substituted by halogen, hydroxyl, cyano, —NR⁸R⁹, NO₂, C1-C3 alkyl, C1-C3 alkoxy, C2-C6 alkenyl, or C2-C6 alkynyl; or the 6-10-membered aryl is optionally substituted by halogen, hydroxyl, cyano, —NR⁸R⁹, or NO₂ substituted C1-C3 alkyl, C1-C3 alkoxy, C2-C6 alkenyl, or C2-C6 alkynyl.

R² and R³ are independently selected from 6-10-membered aryl or 9-18-membered benzoheterocyclyl; the 6-10-membered aryl or 9-18-membered benzoheterocyclyl is optionally substituted by halogen, hydroxyl, cyano, —NR⁸R⁹, NO₂, —NR¹⁰C(O)R¹¹, C1-C6 alkyl, (C1-C6 alkyl)-O—, —C(O)N¹⁰R¹¹, or 5-7-membered heteroaryl; and the C1-C6 alkyl, (C1-C6 alkyl)-O—, or 5-7-membered heteroaryl is optionally substituted by halogen, cyano, hydroxyl, C1-C3 alkyl, (C1-C3 alkyl)-O—, or —NR⁸R⁹.

In some embodiments, R² and R³ are independently selected from 6-10-membered aryl or 9-18-membered benzoheterocyclyl; the 6-10-membered aryl or 9-18-membered benzoheterocyclyl is optionally substituted by halogen, hydroxyl, cyano, —NR⁸R⁹, NO₂, —NR¹⁰C(O)R¹¹, C1-C6 alkyl, (C1-C6 alkyl)-O—, —C(O)NR¹⁰R¹¹, or 5-7-membered heteroaryl; and the C1-C6 alkyl or 5-7-membered heteroaryl is optionally substituted by halogen, cyano, hydroxyl, C1-C3 alkyl, (C1-C3 alkyl)-O—, or —NR⁸R⁹.

R⁸, R⁹, R¹⁰, and R¹¹ are independently selected from H or C1-C6 alkyl.

Provided that at most 2 of W, X, Y, and Z are simultaneously N.

In some embodiments, X is selected from CR⁴.

In some embodiments, Y is selected from CR⁵.

In some embodiments, Z is selected from CR⁶.

In some embodiments, W is N.

In some embodiments, R¹, R⁴, R⁵, R⁶, and R⁷ are independently selected from hydrogen, halogen, hydroxyl, NH₂, (C1-C6 alkyl)-NR⁸—, (C1-C6 alkyl)-O—, C1-C6 alkyl, C3-C6 cycloalkyl, or 6-10-membered aryl, wherein the C1-C6 alkyl, (C1-C6 alkyl)-NR⁸—, (C1-C6 alkyl)-O—, or C3-C6 cycloalkyl is optionally substituted by halogen, cyano, hydroxyl, or —NR⁸R⁹; and the 6-10-membered aryl is optionally substituted by halogen substituted C1-C3 alkoxy.

In some embodiments, R¹, R⁴, R⁵, R⁶, and R⁷ are independently selected from hydrogen, halogen, hydroxyl, NH₂, (C1-C6 alkyl)-NR⁸—, (C1-C6 alkyl)-O—, C1-C6 alkyl, C3-C6 cycloalkyl, or 6-10-membered aryl, wherein the C1-C6 alkyl or C3-C6 cycloalkyl is optionally substituted by halogen, cyano, hydroxyl, or —NR⁸R⁹; and the 6-10-membered aryl is optionally substituted by halogen substituted C1-C3 alkoxy.

In some embodiments, R¹, R⁴, R⁵, R⁶, and R⁷ are independently selected from hydrogen, halogen, hydroxyl, NH₂, (C1-C6 alkyl)-NR⁸—, (C1-C6 alkyl)-O—, C1-C6 alkyl, C3-C6 cycloalkyl, or 6-10-membered aryl, wherein the C1-C6 alkyl, (C1-C6 alkyl)-NR⁸—, (C1-C6 alkyl)-O—, or C3-C6 cycloalkyl is optionally substituted by halogen; and the 6-10-membered aryl is optionally substituted by halogen substituted C1-C3 alkoxy.

In some typical embodiments, R¹, R⁴, R⁵, R⁶, and R⁷ are independently selected from hydrogen, halogen, hydroxyl, NH₂, (C1-C6 alkyl)-NR⁸—, (C1-C6 alkyl)-O—, C1-C6 alkyl, C3-C6 cycloalkyl, or 6-10-membered aryl, wherein the C1-C6 alkyl or C3-C6 cycloalkyl is optionally substituted by halogen; and the 6-10-membered aryl is optionally substituted by halogen substituted C1-C3 alkoxy.

In some embodiments, R¹, R⁴, R⁵, R⁶, and R⁷ are independently selected from hydrogen, halogen, hydroxyl, NH₂, (C1-C6 alkyl)-NR⁸—, (C1-C6 alkyl)-O—, C1-C6 alkyl, C3-C6 cycloalkyl, or 6-10-membered aryl, wherein the C1-C6 alkyl, (C1-C6 alkyl)-NR⁸—, (C1-C6 alkyl)-O—, or C3-C6 cycloalkyl is optionally substituted by fluorine; and the 6-10-membered aryl is optionally substituted by fluorine substituted C1-C3 alkoxy.

In some typical embodiments, R¹, R⁴, R⁵, R⁶, and R⁷ are independently selected from hydrogen, halogen, hydroxyl, NH₂, (C1-C6 alkyl)-NR⁸—, (C1-C6 alkyl)-O—, C1-C6 alkyl, C3-C6 cycloalkyl, or 6-10-membered aryl, wherein the C1-C6 alkyl or C3-C6 cycloalkyl is optionally substituted by fluorine; and the 6-10-membered aryl is optionally substituted by fluorine substituted C1-C3 alkoxy.

In some more typical embodiments, R¹ is hydrogen.

In some more typical embodiments, R⁴ is selected from hydrogen, C1-C6 alkyl, or C3-C6 cycloalkyl; preferably, R⁴ is selected from hydrogen, cyclopropyl, or C1-C6 alkyl; and preferably, R⁴ is selected from hydrogen or C1-C6 alkyl.

In some more typical embodiments, R⁴ is selected from hydrogen, methyl, or cyclopropyl; and preferably, R⁴ is selected from hydrogen or methyl.

In some more typical embodiments, R⁵ is selected from hydrogen or C1-C6 alkyl.

In some more typical embodiments, R⁵ is selected from hydrogen or methyl; and preferably, R⁵ is hydrogen.

In some more typical embodiments, R⁶ is selected from hydrogen, chlorine, hydroxyl, cyclopropyl, CF₃CH₂O—, CHF₂O—, CF₃CH₂NH—, 4-difluoromethoxylphenyl, or CH₃CH₂O—; preferably, R⁶ is selected from cyclopropyl, CF₃CH₂O—, CF₃CH₂NH—, or CH₃CH₂O—; and more preferably, R⁶ is selected from cyclopropyl, CF₃CH₂O—, or CF₃CH₂NH—.

In some more typical embodiments, R⁷ is hydrogen.

In some embodiments, R² and R³ are independently selected from phenyl, naphthyl,

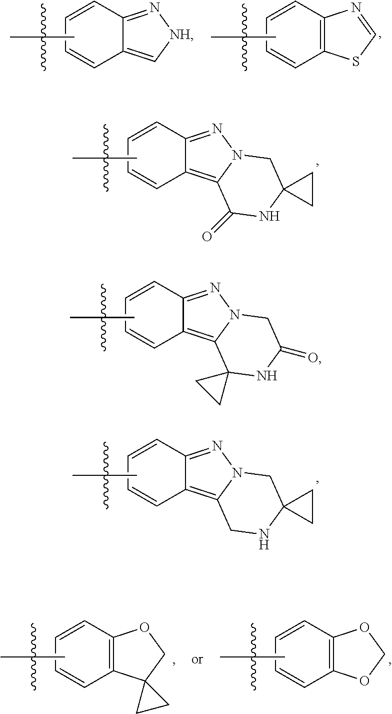

wherein the group is optionally substituted by halogen, hydroxyl, cyano, —NR⁸R⁹, NO₂, —NR¹⁰C(O)R¹¹, C1-C6 alkyl, (C1-C6 alkyl)-O—, —C(O)NR¹⁰R¹¹, or 5-7-membered heteroaryl; and the C1-C6 alkyl, (C1-C6 alkyl)-O—, or 5-7-membered heteroaryl is optionally substituted by halogen, cyano, hydroxyl, C1-C3 alkyl, (C1-C3 alkyl)-O—, or —NR⁸R⁹.

In some embodiments, R² and R³ are independently selected from phenyl, naphthyl,

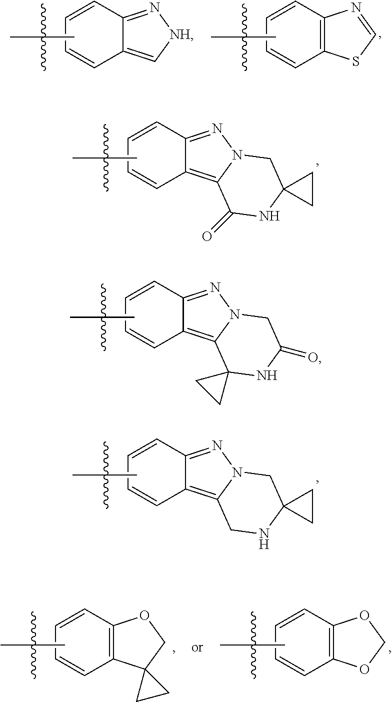

wherein the group is optionally substituted by halogen, hydroxyl, cyano, —NR⁸R⁹, NO₂, —NR¹⁰C(O)R¹¹, C1-C6 alkyl, (C1-C6 alkyl)-O—, —C(O)NR¹⁰R¹¹, or 5-7-membered heteroaryl; and the C1-C6 alkyl or 5-7-membered heteroaryl is optionally substituted by halogen, cyano, hydroxyl, C1-C3 alkyl, (C1-C3 alkyl)-O—, or —NR⁸R⁹.

In some embodiments, R² and R³ are independently selected from phenyl, naphthyl

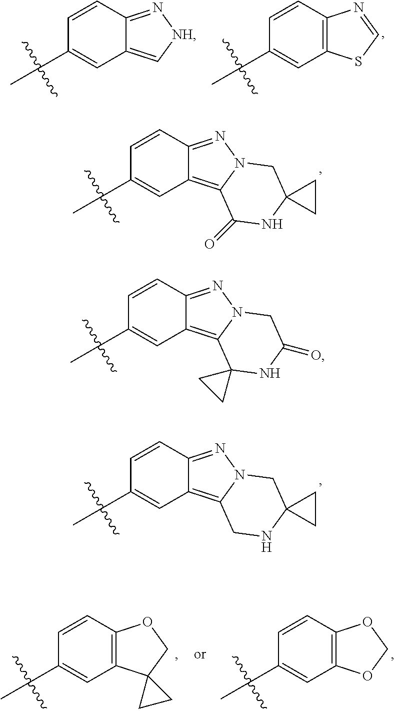

wherein the group is optionally substituted by halogen, hydroxyl, cyano, —NR⁸R⁹, NO₂, —NR¹⁰C(O)R¹¹, C1-C6 alkyl, (C1-C6 alkyl)-O—, —C(O)NR¹⁰R¹¹, or 5-7-membered heteroaryl; and the C1-C6 alkyl, (C1-C6 alkyl)-O—, or 5-7-membered heteroaryl is optionally substituted by halogen, cyano, hydroxyl, C1-C3 alkyl, (C1-C3 alkyl)-O—, or —NR⁸R⁹.

In some embodiments, R² and R³ are independently selected from phenyl, naphthyl,

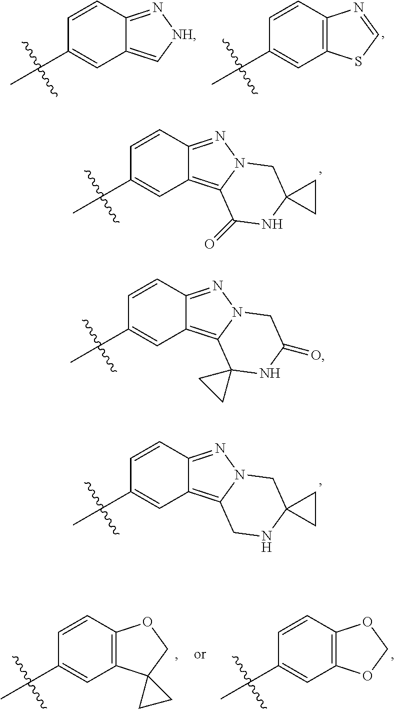

wherein the group is optionally substituted by halogen, hydroxyl, cyano, —NR⁸R⁹, NO₂, —NR¹⁰C(O)R¹¹, C1-C6 alkyl, (C1-C6 alkyl)-O—, —C(O)NR¹⁰R¹¹, or 5-7-membered heteroaryl; and the C1-C6 alkyl or 5-7-membered heteroaryl is optionally substituted by halogen, cyano, hydroxyl, C1-C3 alkyl, (C1-C3 alkyl)-O—, or —NR⁸R⁹.

In some embodiments, R² and R³ are independently selected from phenyl, naphthyl,

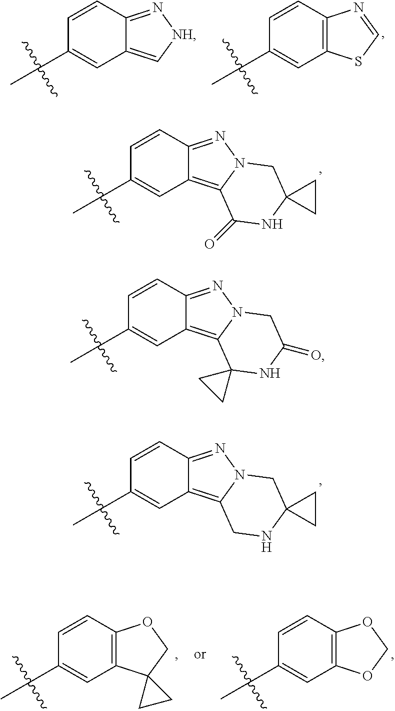

wherein the group is optionally substituted by halogen, cyano, —NR⁸R⁹, —NR¹⁰C(O)R¹¹, C1-C6 alkyl, (C1-C6 alkyl)-O—, or 5-7-membered heteroaryl; and the C1-C6 alkyl, (C1-C6 alkyl)-O—, or 5-7-membered heteroaryl is optionally substituted by methyl, halogen, or cyano.

In some embodiments, R² and R³ are independently selected from phenyl naphthyl,

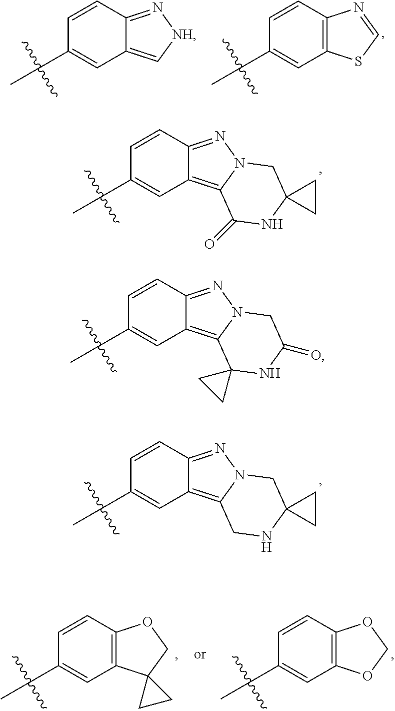

wherein the group is optionally substituted by halogen, cyano, —NR⁸R⁹, —NR¹⁰C(O)R¹¹, C1-C6 alkyl, (C1-C6 alkyl)-O—, or 5-7-membered heteroaryl; and the C1-C6 alkyl or 5-7-membered heteroaryl is optionally substituted by halogen or cyano.

In some embodiments, R⁸, R⁹, R¹⁰, and R¹¹ are independently selected from H or C1-C6 alkyl.

In some embodiments, R⁸, R⁹, and R¹⁰ are independently H.

In some embodiments, R¹¹ is selected from C1-C6 alkyl.

In some embodiments, R¹¹ is methyl.

In some embodiments, R² is selected from phenyl,

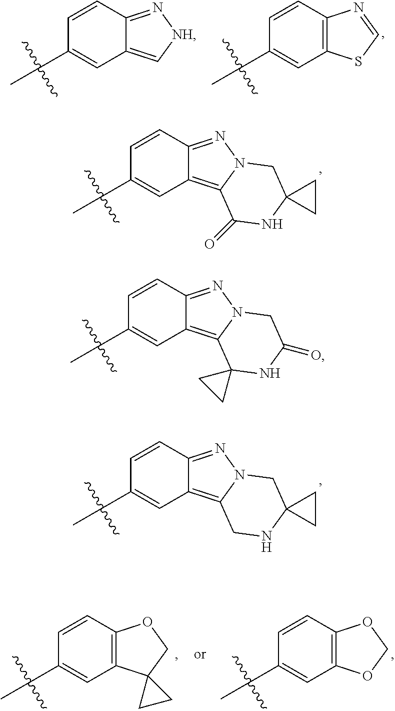

wherein the group is optionally substituted by difluoromethoxy, methyl, NH₂, methoxy, fluorine, cyanomethyl, CH₃CONH—, 1-methyl-1H-imidazol-4-yl, or 1-methyl-1H-pyrazol-5-yl.

In some embodiments, R² is selected from phenyl,

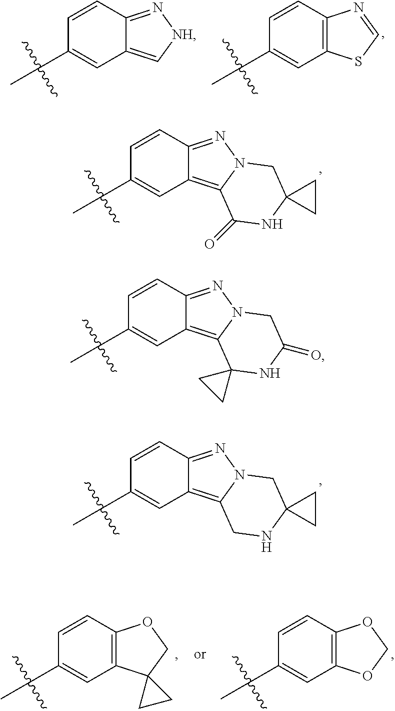

wherein the group is optionally substituted by difluoromethoxy, methyl, NH₂, methoxy, fluorine, cyanomethyl, or 1-methyl-1H-pyrazol-5-yl

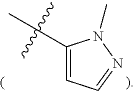 [SMILES: *c1ccnn1C]

In some typical embodiments, R² is selected from

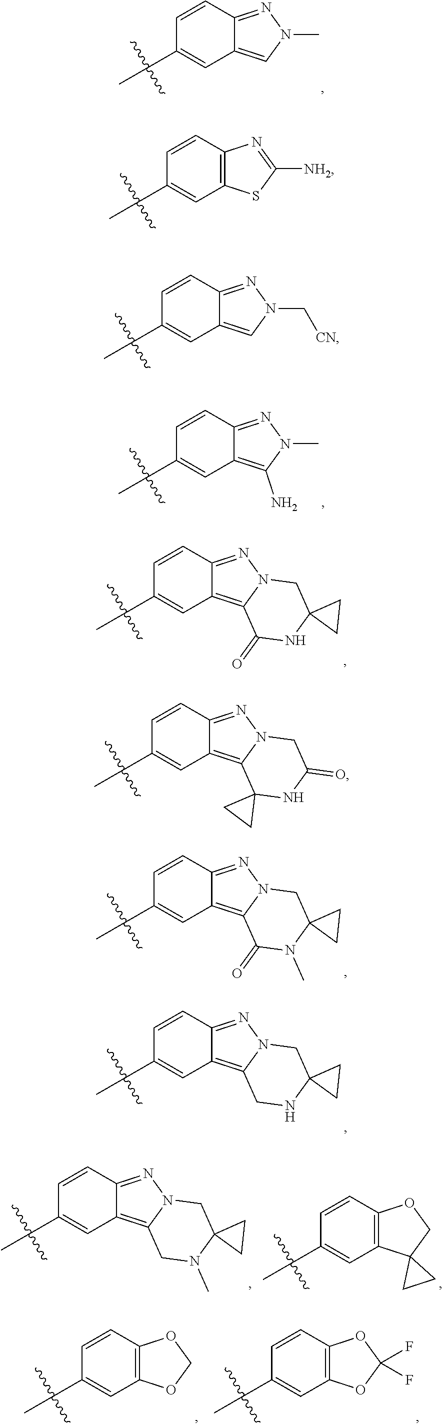

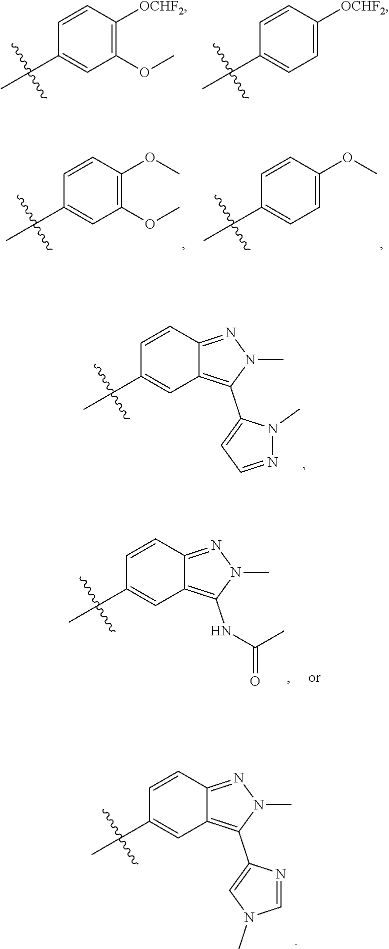

In some embodiments, R² is selected from

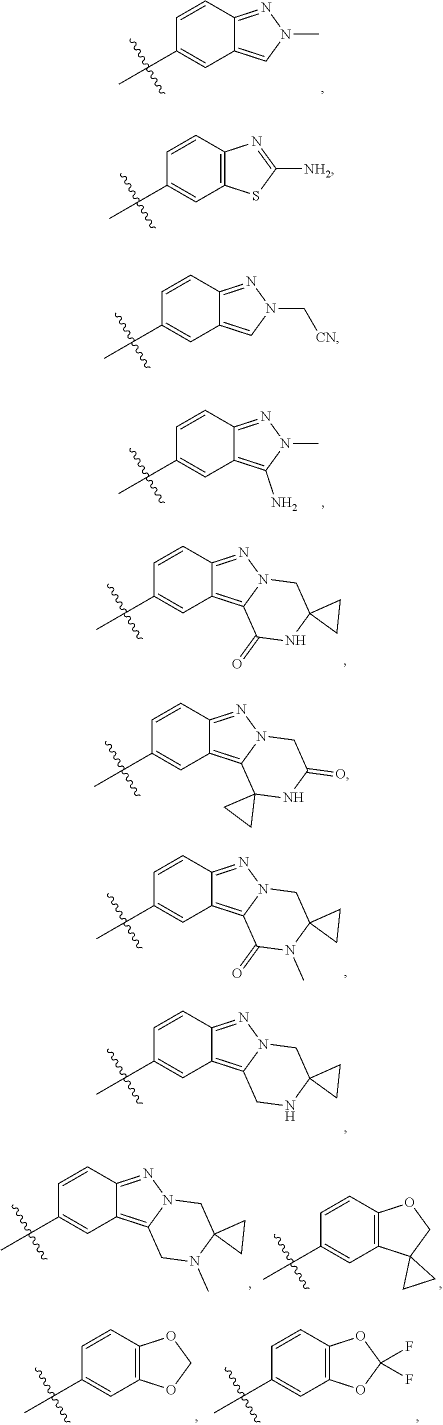

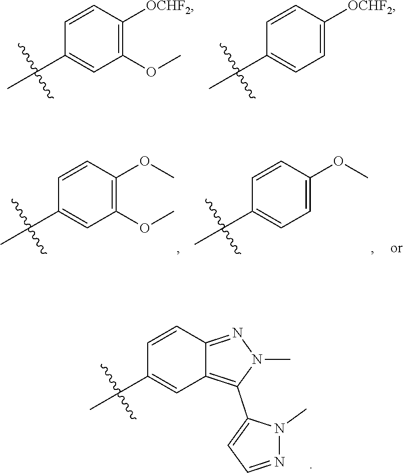

Preferably, R² is selected from

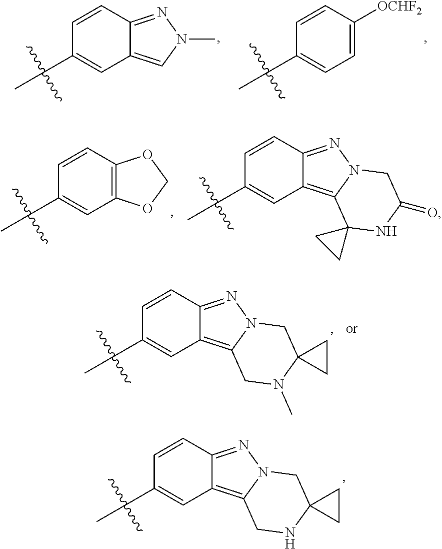 [SMILES: *c1ccc2nn(C)cc2c1 ; *c1ccc(OC(F)F)cc1 ; *c1ccc2c(c1)OCO2 ; *c1ccc2nn3c(c2c1)C1(CC1)NC(=O)C3 ; *c1ccc2nn3c(c2c1)CN(C)C1(CC1)C3 ; *c1ccc2nn3c(c2c1)CNC1(CC1)C3]

and more preferably, R² is selected from

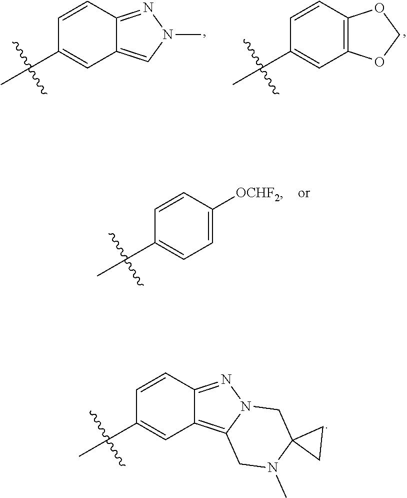 [SMILES: *c1ccc2nn(C)cc2c1 ; *c1ccc2c(c1)OCO2 ; *c1ccc(OC(F)F)cc1 ; *c1ccc2nn3c(c2c1)CN(C)C1(CC1)C3]

In some embodiments, R³ is selected from phenyl,

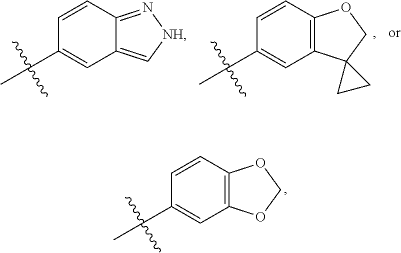 [SMILES: *c1ccc2n[nH]cc2c1 ; *c1ccc2c(c1)C1(CC1)CO2 ; *c1ccc2c(c1)OCO2]

wherein the group is optionally substituted by difluoromethoxy, methyl, NH₂, fluorine, methoxy, CH₃CONH—, 1-methyl-1H-imidazol-4-yl, or 1-methyl-1H-pyrazol-5-yl.

In some embodiments, R³ is selected from phenyl,

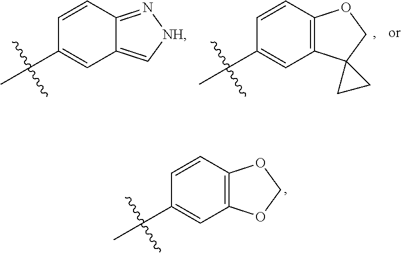 [SMILES: *c1ccc2n[nH]cc2c1 ; *c1ccc2c(c1)C1(CC1)CO2 ; *c1ccc2c(c1)OCO2]

wherein the group is optionally substituted by difluoromethoxy, methyl, NH₂, fluorine, methoxy, or 1-methyl-1H-pyrazol-5-yl.

In some embodiments, R³ is selected from

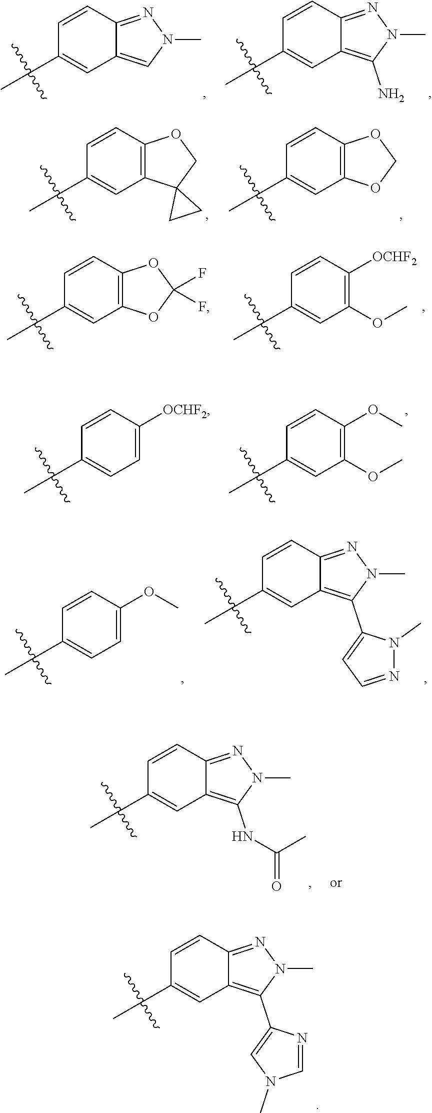

In some embodiments, R³ is selected from

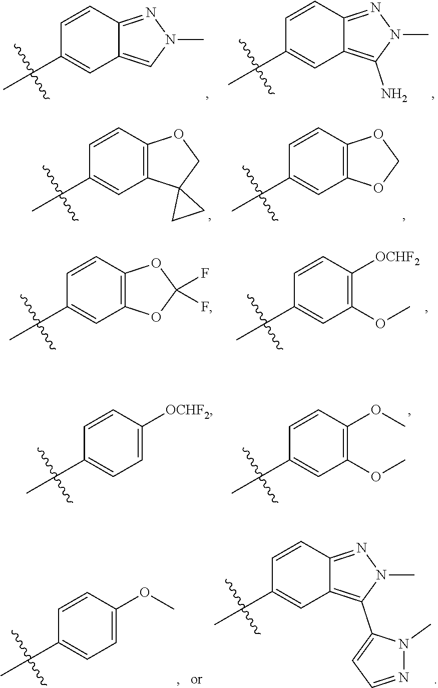

Preferably, R is selected from

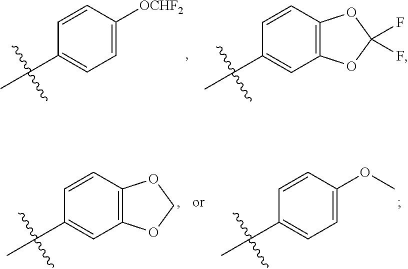

and more preferably, R³ is selected from

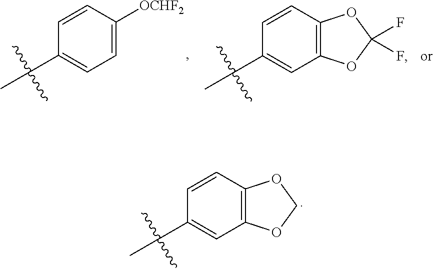 [SMILES: *c1ccc(OC(F)F)cc1 ; *c1ccc2c(c1)OC(F)(F)O2 ; *c1ccc2c(c1)OCO2]

In some embodiments, the foregoing compound of formula I has a structure of formula II,

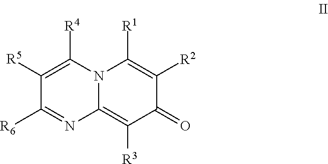

wherein R¹, R², R³, R⁴, R⁵, R⁶, R⁷, R⁸, R⁹, R¹⁰, and R¹¹ are defined as the compound of formula I.

In some embodiments, the foregoing compound of formula I has a structure of formula III,

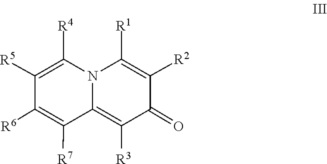

wherein R¹, R², R³, R⁴, R⁵, R⁶, R⁷, R⁸, R⁹, R¹⁰, and R¹¹ are defined as the compound of formula I.

In another aspect, the present invention provides the following compounds or a pharmaceutically acceptable salts thereof:

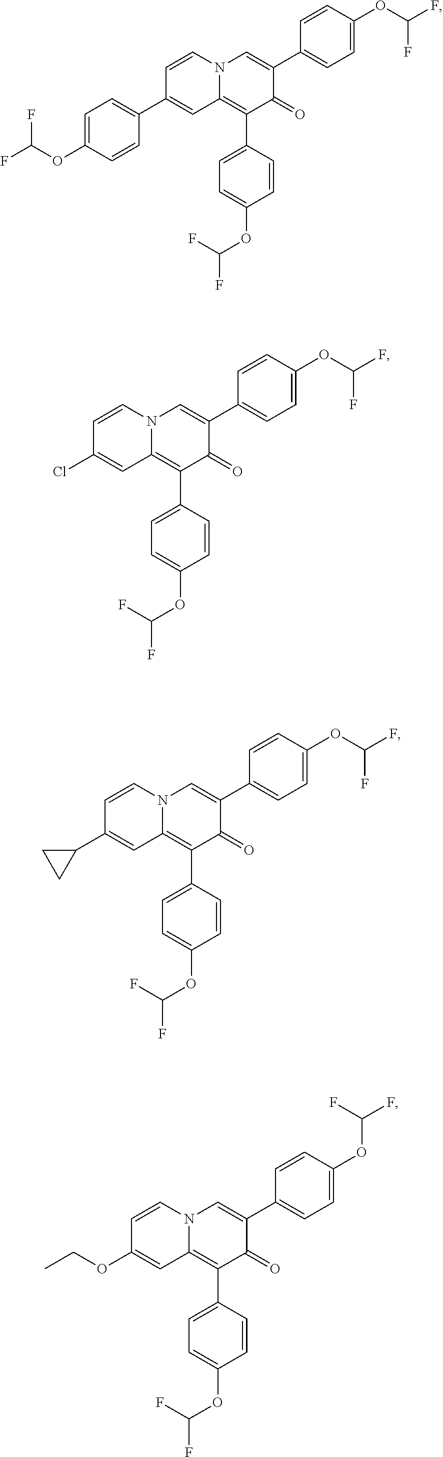 [SMILES: O=c1c(-c2ccc(OC(F)F)cc2)cn2ccc(-c3ccc(OC(F)F)cc3)cc2c1-c1ccc(OC(F)F)cc1 ; O=c1c(-c2ccc(OC(F)F)cc2)cn2ccc(Cl)cc2c1-c1ccc(OC(F)F)cc1 ; O=c1c(-c2ccc(OC(F)F)cc2)cn2ccc(C3CC3)cc2c1-c1ccc(OC(F)F)cc1 ; CCOc1ccn2cc(-c3ccc(OC(F)F)cc3)c(=O)c(-c3ccc(OC(F)F)cc3)c2c1]

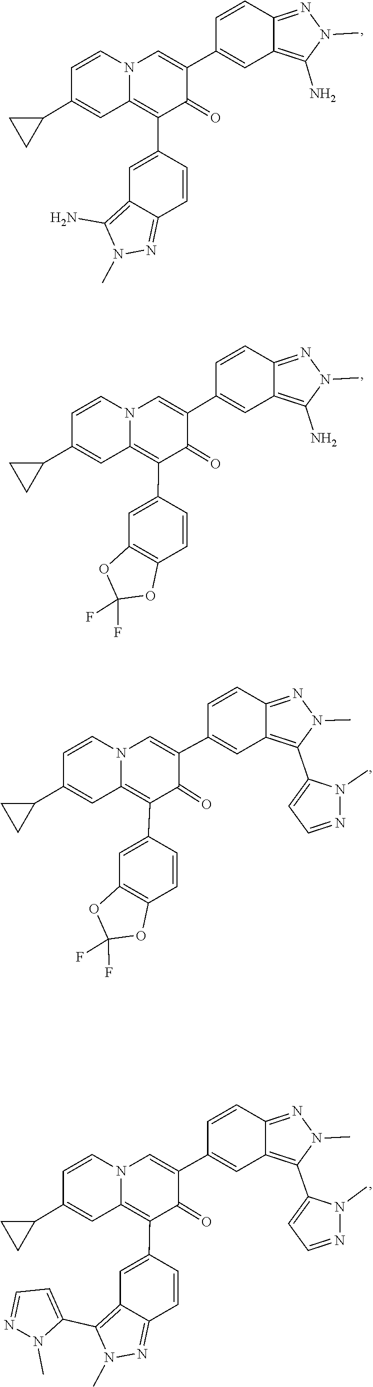 [SMILES: Cn1nc2ccc(-c3cn4ccc(C5CC5)cc4c(-c4ccc5nn(C)c(N)c5c4)c3=O)cc2c1N ; Cn1nc2ccc(-c3cn4ccc(C5CC5)cc4c(-c4ccc5c(c4)OC(F)(F)O5)c3=O)cc2c1N ; Cn1nccc1-c1c2cc(-c3cn4ccc(C5CC5)cc4c(-c4ccc5c(c4)OC(F)(F)O5)c3=O)ccc2nn1C ; Cn1nccc1-c1c2cc(-c3cn4ccc(C5CC5)cc4c(-c4ccc5nn(C)c(-c6ccnn6C)c5c4)c3=O)ccc2nn1C]

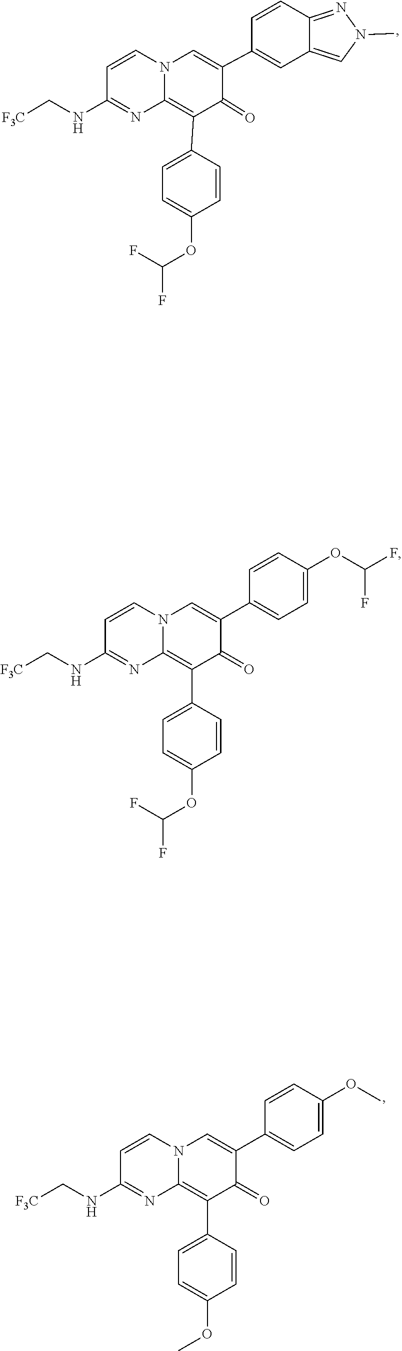 [SMILES: Cn1cc2cc(-c3cn4ccc(NCC(F)(F)F)nc4c(-c4ccc(OC(F)F)cc4)c3=O)ccc2n1 ; O=c1c(-c2ccc(OC(F)F)cc2)cn2ccc(NCC(F)(F)F)nc2c1-c1ccc(OC(F)F)cc1 ; COc1ccc(-c2cn3ccc(NCC(F)(F)F)nc3c(-c3ccc(OC)cc3)c2=O)cc1]

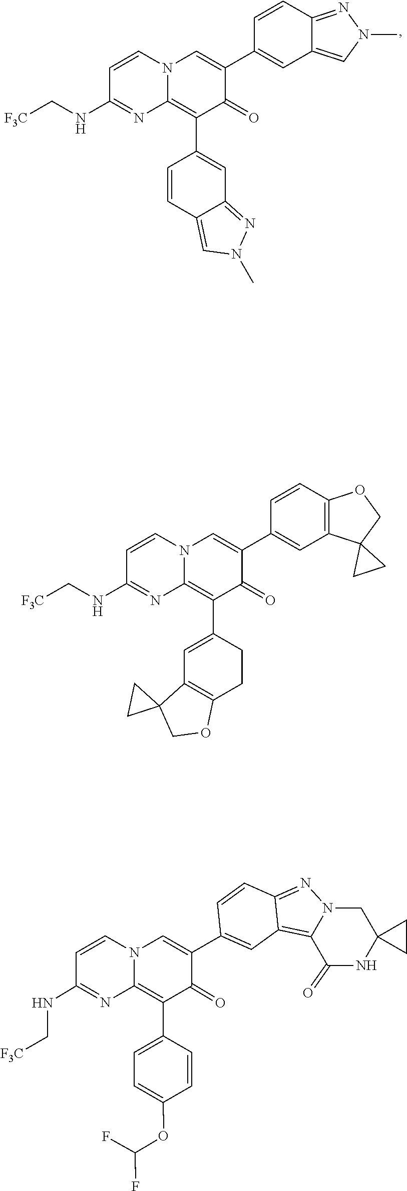 [SMILES: Cn1cc2cc(-c3cn4ccc(NCC(F)(F)F)nc4c(-c4ccc5cn(C)nc5c4)c3=O)ccc2n1 ; O=c1c(-c2ccc3c(c2)C2(CC2)CO3)cn2ccc(NCC(F)(F)F)nc2c1C1=CC2=C(CC1)OCC21CC1 ; O=C1NC2(CC2)Cn2nc3ccc(-c4cn5ccc(NCC(F)(F)F)nc5c(-c5ccc(OC(F)F)cc5)c4=O)cc3c21]

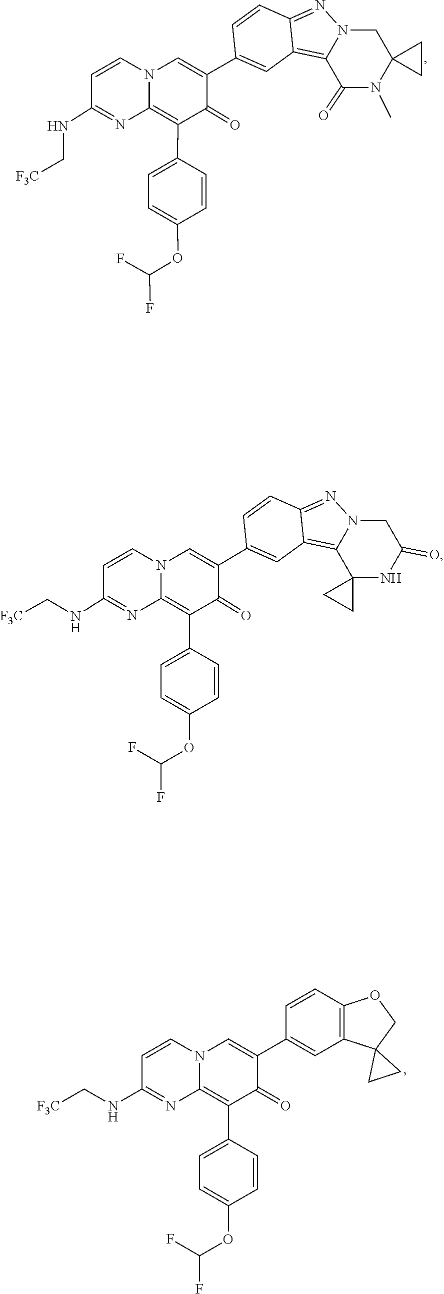 [SMILES: CN1C(=O)c2c3cc(-c4cn5ccc(NCC(F)(F)F)nc5c(-c5ccc(OC(F)F)cc5)c4=O)ccc3nn2CC12CC2 ; O=C1Cn2nc3ccc(-c4cn5ccc(NCC(F)(F)F)nc5c(-c5ccc(OC(F)F)cc5)c4=O)cc3c2C2(CC2)N1 ; O=c1c(-c2ccc3c(c2)C2(CC2)CO3)cn2ccc(NCC(F)(F)F)nc2c1-c1ccc(OC(F)F)cc1]

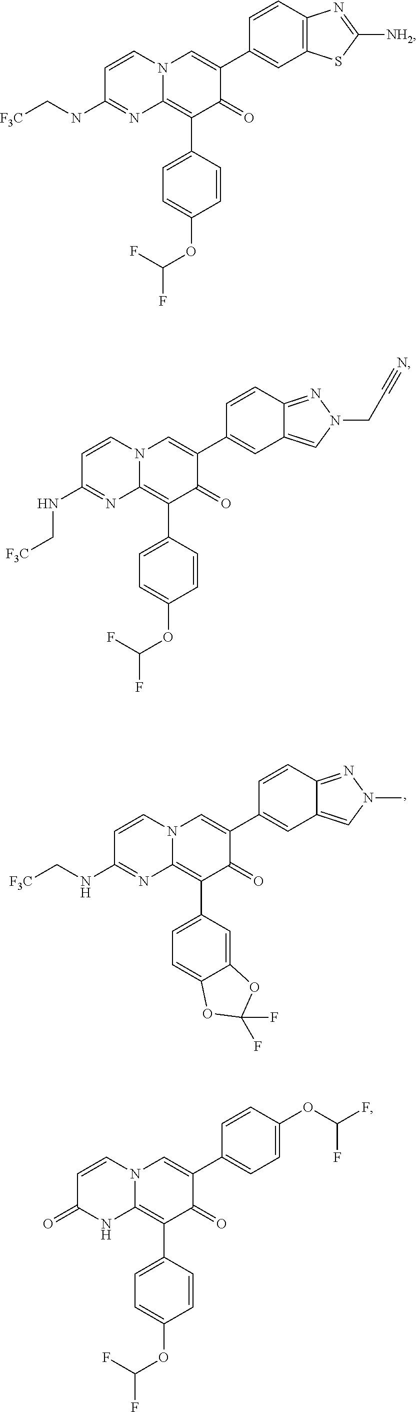

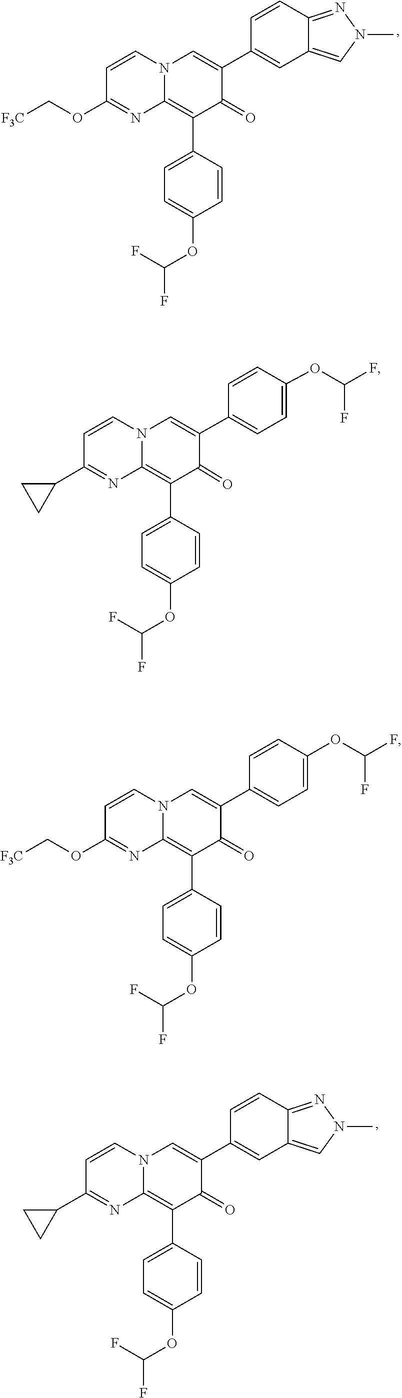 [SMILES: Cn1cc2cc(-c3cn4ccc(OCC(F)(F)F)nc4c(-c4ccc(OC(F)F)cc4)c3=O)ccc2n1 ; O=c1c(-c2ccc(OC(F)F)cc2)cn2ccc(C3CC3)nc2c1-c1ccc(OC(F)F)cc1 ; O=c1c(-c2ccc(OC(F)F)cc2)cn2ccc(OCC(F)(F)F)nc2c1-c1ccc(OC(F)F)cc1 ; Cn1cc2cc(-c3cn4ccc(C5CC5)nc4c(-c4ccc(OC(F)F)cc4)c3=O)ccc2n1]

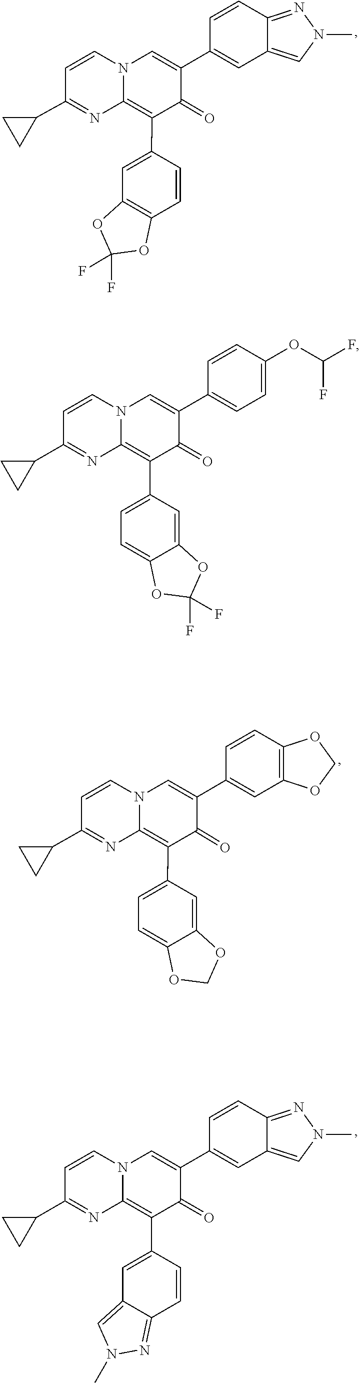 [SMILES: Cn1cc2cc(-c3cn4ccc(C5CC5)nc4c(-c4ccc5c(c4)OC(F)(F)O5)c3=O)ccc2n1 ; O=c1c(-c2ccc(OC(F)F)cc2)cn2ccc(C3CC3)nc2c1-c1ccc2c(c1)OC(F)(F)O2 ; O=c1c(-c2ccc3c(c2)OCO3)cn2ccc(C3CC3)nc2c1-c1ccc2c(c1)OCO2 ; Cn1cc2cc(-c3cn4ccc(C5CC5)nc4c(-c4ccc5nn(C)cc5c4)c3=O)ccc2n1]

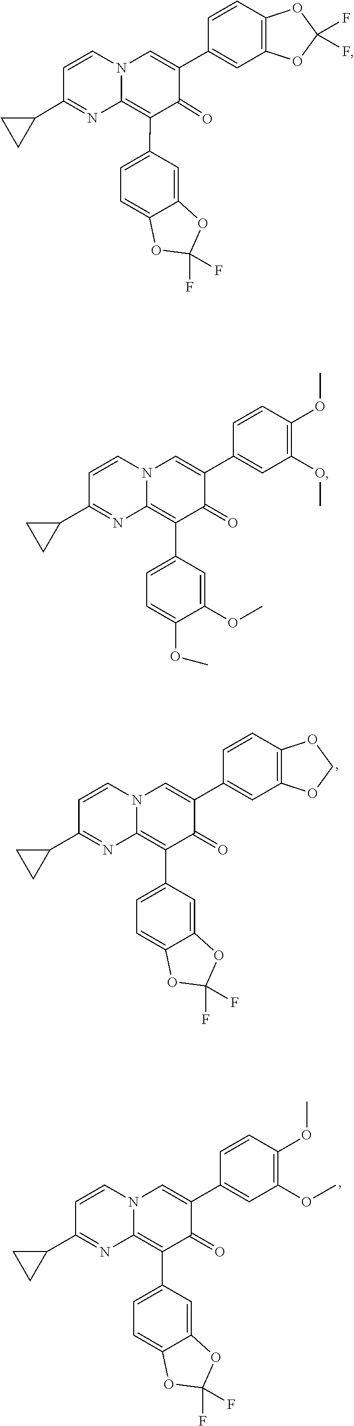 [SMILES: O=c1c(-c2ccc3c(c2)OC(F)(F)O3)cn2ccc(C3CC3)nc2c1-c1ccc2c(c1)OC(F)(F)O2 ; COc1ccc(-c2cn3ccc(C4CC4)nc3c(-c3ccc(OC)c(OC)c3)c2=O)cc1OC ; O=c1c(-c2ccc3c(c2)OCO3)cn2ccc(C3CC3)nc2c1-c1ccc2c(c1)OC(F)(F)O2 ; COc1ccc(-c2cn3ccc(C4CC4)nc3c(-c3ccc4c(c3)OC(F)(F)O4)c2=O)cc1OC]

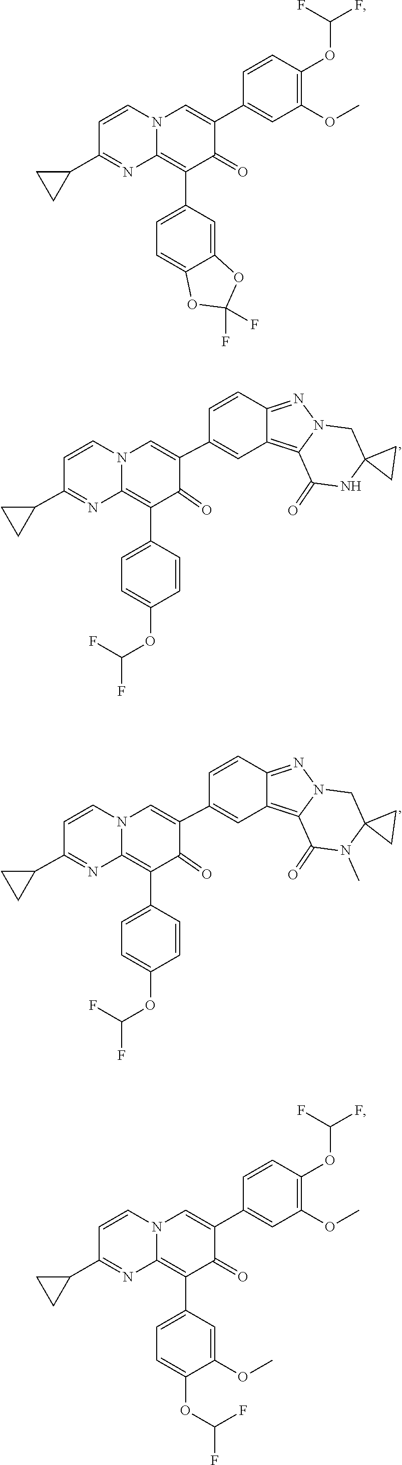 [SMILES: COc1cc(-c2cn3ccc(C4CC4)nc3c(-c3ccc4c(c3)OC(F)(F)O4)c2=O)ccc1OC(F)F ; O=C1NC2(CC2)Cn2nc3ccc(-c4cn5ccc(C6CC6)nc5c(-c5ccc(OC(F)F)cc5)c4=O)cc3c21 ; CN1C(=O)c2c3cc(-c4cn5ccc(C6CC6)nc5c(-c5ccc(OC(F)F)cc5)c4=O)ccc3nn2CC12CC2 ; COc1cc(-c2cn3ccc(C4CC4)nc3c(-c3ccc(OC(F)F)c(OC)c3)c2=O)ccc1OC(F)F]

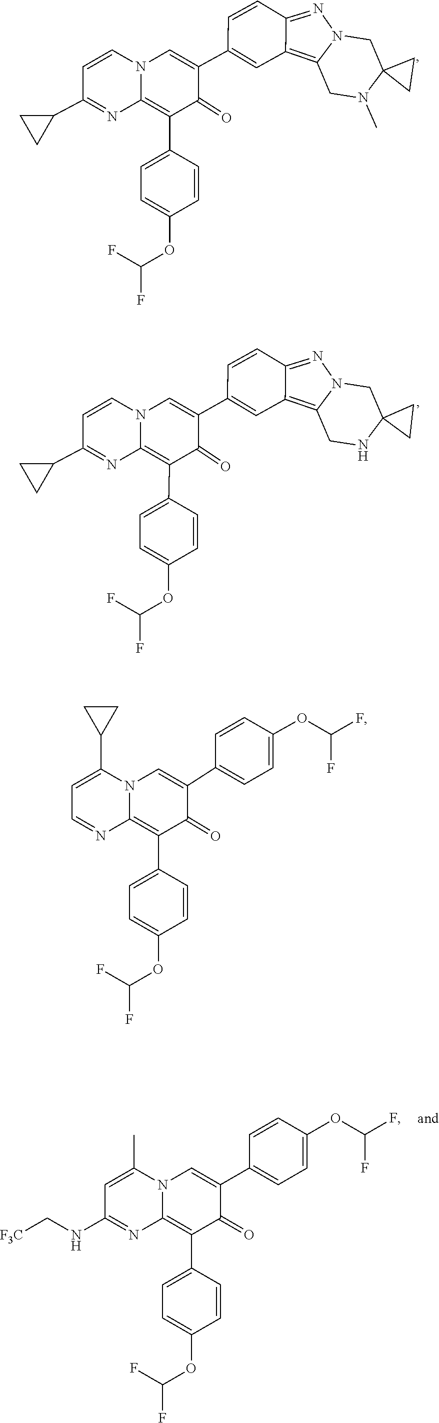 [SMILES: CN1Cc2c3cc(-c4cn5ccc(C6CC6)nc5c(-c5ccc(OC(F)F)cc5)c4=O)ccc3nn2CC12CC2 ; O=c1c(-c2ccc3nn4c(c3c2)CNC2(CC2)C4)cn2ccc(C3CC3)nc2c1-c1ccc(OC(F)F)cc1 ; O=c1c(-c2ccc(OC(F)F)cc2)cn2c(C3CC3)ccnc2c1-c1ccc(OC(F)F)cc1 ; Cc1cc(NCC(F)(F)F)nc2c(-c3ccc(OC(F)F)cc3)c(=O)c(-c3ccc(OC(F)F)cc3)cn12]

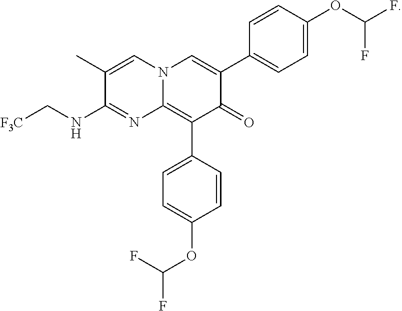 [SMILES: Cc1cn2cc(-c3ccc(OC(F)F)cc3)c(=O)c(-c3ccc(OC(F)F)cc3)c2nc1NCC(F)(F)F]

In some embodiments, the present invention provides a pharmaceutical composition, comprising a therapeutically effective amount of the compound of formula I, formula II, or formula III, or a pharmaceutically acceptable salt thereof, and a pharmaceutically acceptable carrier.

In some embodiments, the present invention provides a method for treating a disease or condition mediated by overexpression of MAT2A in mammals in need, comprising administering an effective amount of the compound of formula I, formula II, or formula III or a pharmaceutically acceptable salt thereof.

In some embodiments, the present invention provides a method for treating a MTAP-deleted (null) cancer in subjects, comprising administering an effective amount of a compound of formula I, formula II, or formula III or a pharmaceutically acceptable salt thereof.

In some embodiments, the present invention provides a method for inhibiting synthesis of S-adenosylmethionine (SAM) from methionine and ATP in cells through MAT2A, comprising contacting cells with an effective amount of the compound of formula I, formula II, or formula III or a pharmaceutically acceptable salt thereof.

In some embodiments, the present invention provides a method for treating a cancer in subjects with a cancer, wherein the cancer is characterized by a reduction or deletion of methylthioadenosine phosphorylase (MTAP) gene expression, a deletion of a MTAP gene, or a reduction of MTAP protein function, and the method comprises administering a therapeutically effective amount of a compound of formula I, formula II, or formula III or a pharmaceutically acceptable salt thereof to a subject.

In some embodiments, the present invention provides a compound of formula I, formula II, or formula III or a pharmaceutically acceptable salt thereof, for use in the inhibition of the synthesis of S-adenosylmethionine (SAM) from methionine and ATP by MAT2A in cells.

In some embodiments, the present invention provides a compound of formula I, formula II, or formula III or a pharmaceutically acceptable salt thereof, for use in the treatment of a subject suffering from a disease or condition, wherein the disease or condition mediated by overexpression of MAT2A.

In some embodiments, the present invention provides a compound of formula I, formula II, or formula III or a pharmaceutically acceptable salt thereof, for use in the treatment of cancer in a subject suffering from a cancer, wherein the cancer is a reduction or deletion of methylthioadenosine phosphorylase (MTAP) gene expression, a deletion of a MTAP gene, or a reduction of MTAP protein function.

In a further aspect, the present invention provides a method for preparing a compound of formula I, formula II, or formula III, comprising but not limited to the following synthesis schemes:

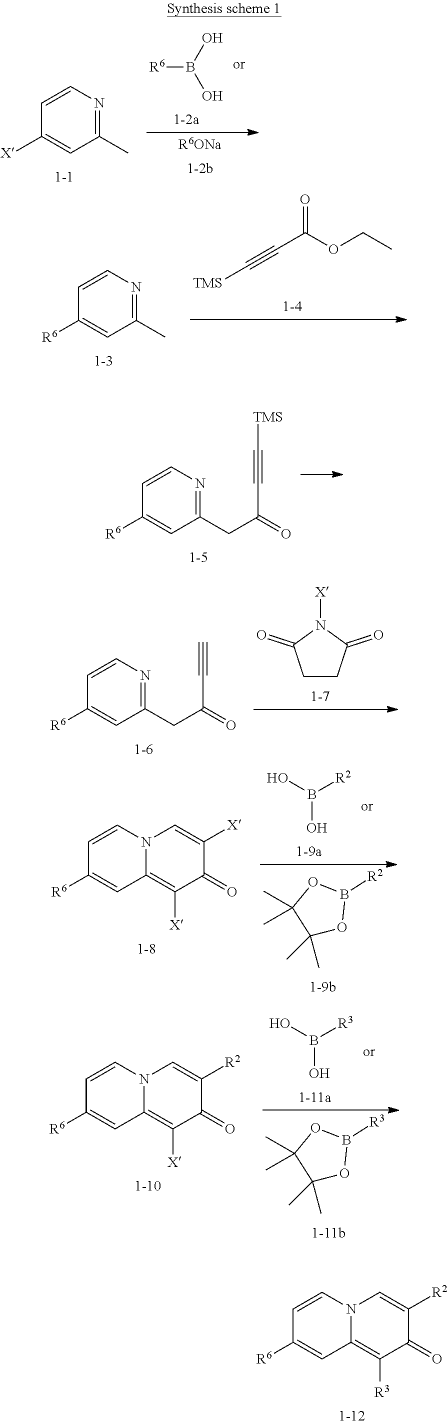

wherein X′ is selected from chlorine, bromine, or iodine; and R⁶, R², and R³ are defined as described in formula I above.

Specifically, a compound of formula 1-1 is condensed with a compound of formula 1-2a or 1-2b under alkaline conditions to obtain a compound of formula 1-3, the compound of formula 1-3 reacts with a compound of formula 1-4 (ethyl 3-(trimethylsilyl) propiolate) to obtain a compound of formula 1-5, trimethylsilyl is removed from the compound of formula 1-5 to obtain a compound of formula 1-6, the compound of formula 1-6 is halogenated under the action of a compound of formula 1-7 as a halogenation reagent to obtain a compound of formula 1-8, the compound of formula 1-8 is condensed with a compound of formula 1-9a or 1-9b under alkaline conditions to obtain a compound of formula 1-10, and the compound of formula 1-10 is condensed with a compound of formula 1-11a or 1-11b under alkaline conditions to obtain a compound of formula 1-12.

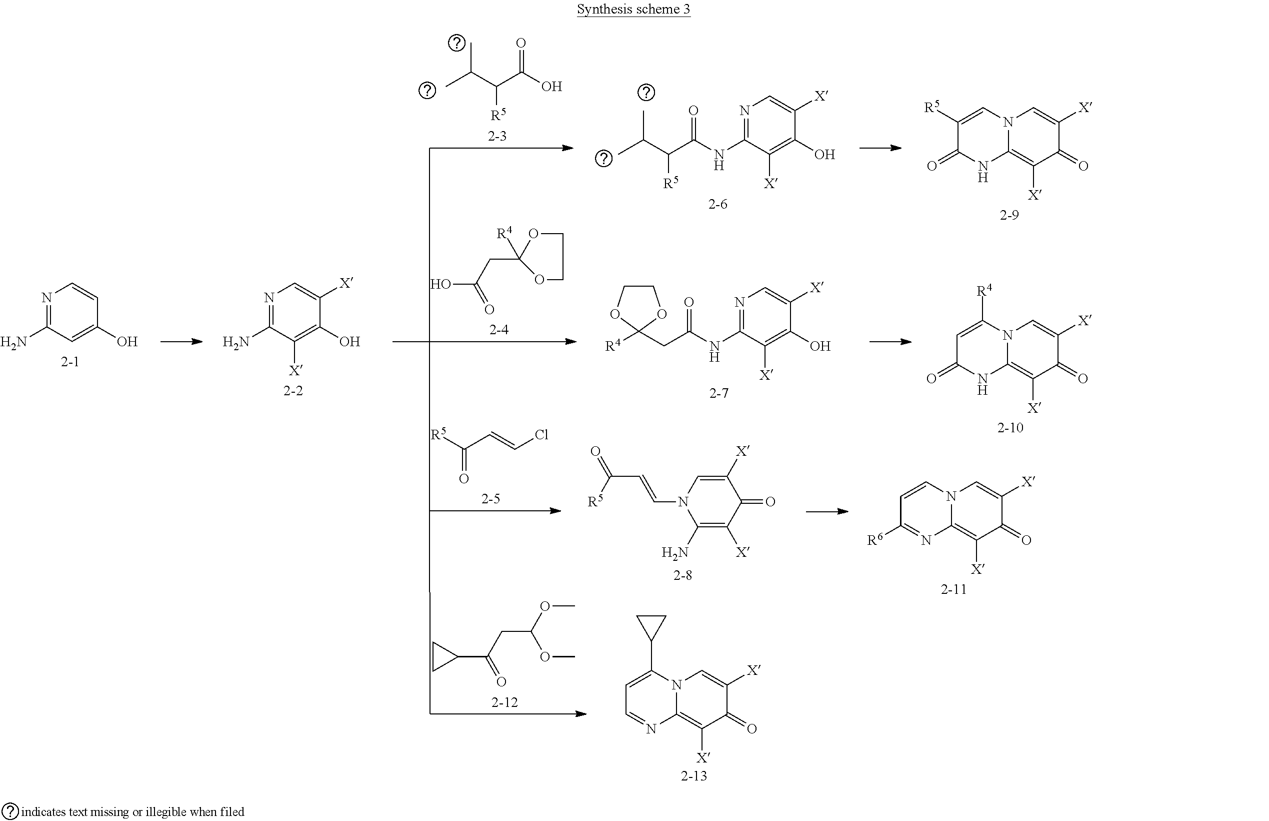

wherein X′ is selected from chlorine, bromine, or iodine; R^{a} is selected from C1-C3 alkyl; and R⁴, R⁵, R⁶, R², and R³ are defined as described in formula I above.

Specifically, a compound of formula 2-1 is halogenated under the action of a halogenation reagent to obtain a compound of formula 2-2, the compound of formula 2-2 is condensed with a compound of formula 2-3 to obtain a compound of formula 2-6, and a compound of formula 2-6 is cyclized under acidic conditions to obtain a compound of formula 2-9; or

A compound of formula 2-2 is condensed with a compound of formula 2-4 to obtain a compound of formula 2-7, the compound of formula 2-7 is cyclized under acidic conditions, and then hydroxyl is removed under acidic conditions to obtain a compound of formula 2-10; or

A compound of formula 2-2 is condensed with a compound of formula 2-5 under alkaline conditions to obtain a compound of formula 2-8, and the compound of formula 2-8 is cyclized under acidic conditions to obtain a compound of formula 2-11; or

A compound of formula 2-2 is cyclized with a compound of formula 2-12 to obtain a compound of formula 2-13.

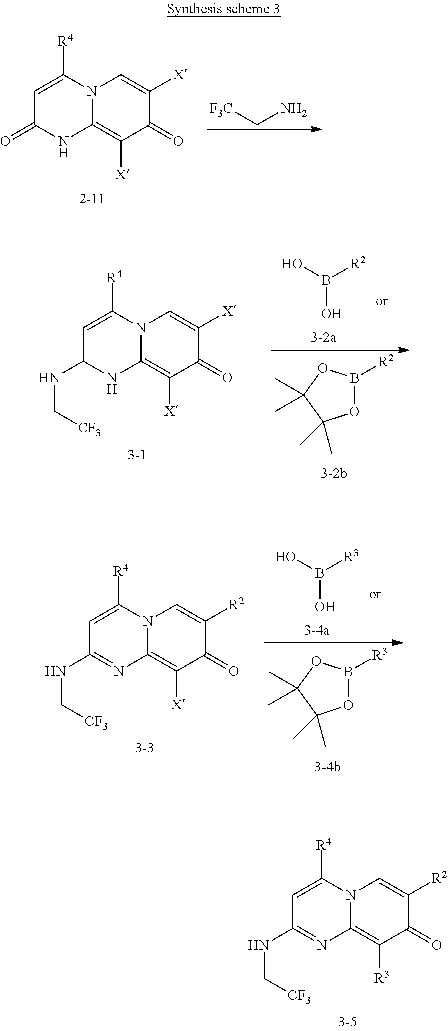

wherein X′ is selected from chlorine, bromine, or iodine; and R⁴, R², and R³ are defined as described in formula I above.

Specifically, a compound of formula 2-10 reacts with trifluoroethylamine to obtain a compound of formula 3-1, the compound of formula 3-1 is condensed with a compound of formula 3-2a or 3-2b under alkaline conditions to obtain a compound of formula 3-3, and the compound of formula 3-3 is condensed with a compound of formula 3-4a or 3-4b under alkaline conditions to obtain a compound of formula 3-5.

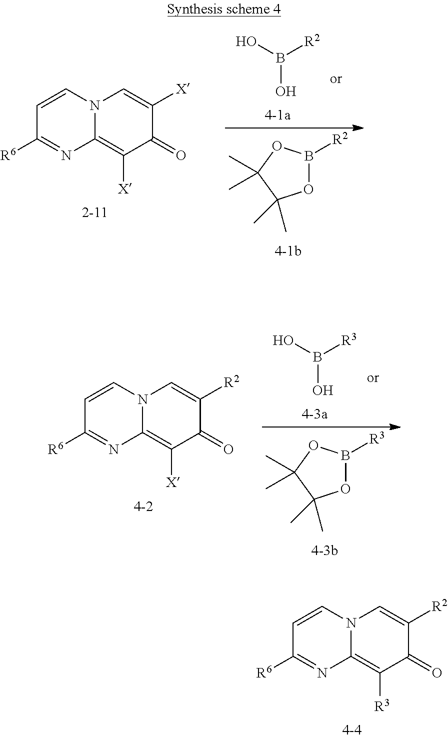

wherein X′ is selected from chlorine, bromine, or iodine; and R⁶, R², and R³ are defined as described in formula I above.

Specifically, a compound of formula 2-11 is condensed with a compound of formula 4-1a or 4-1b under alkaline conditions to obtain a compound of formula 4-2, and the compound of formula 4-2 is condensed with a compound of formula 4-3a or 3-3b under alkaline conditions to obtain a compound of formula 4-4.

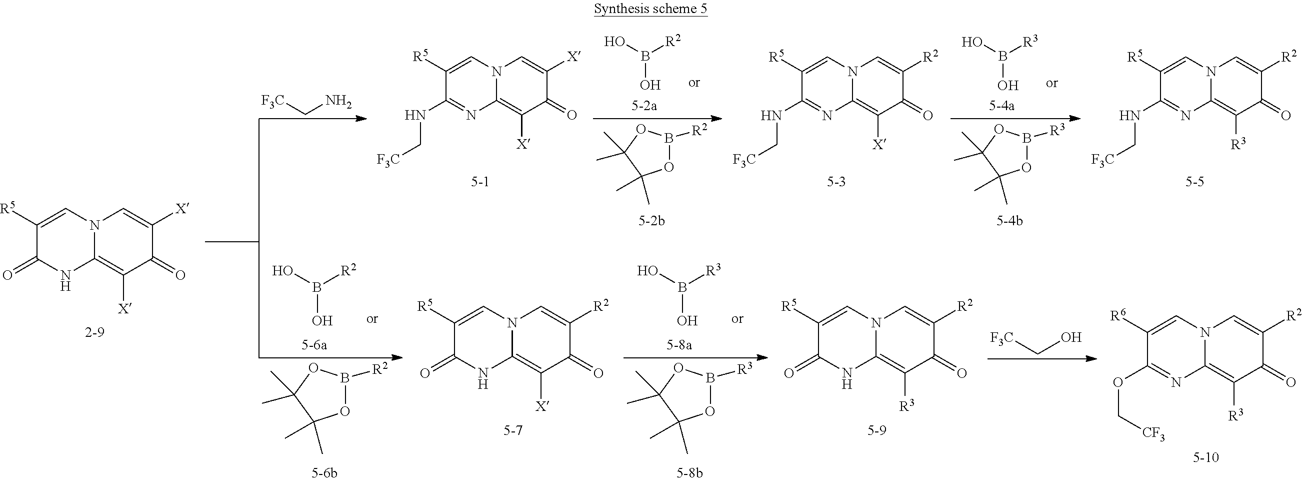

wherein X′ is selected from chlorine, bromine, or iodine; and R⁶, R², R³ and R³ are defined as described in formula I above.

Specifically, a compound of formula 2-9 reacts with trifluoroethylamine under the action of a condensing agent to obtain a compound of formula 5-1, the compound of formula 5-1 is condensed with a compound of formula 5-2a or 5-2b under alkaline conditions to obtain a compound of formula 5-3, and the compound of formula 5-3 is condensed with a compound of formula 5-4a or 5-4b under alkaline conditions to obtain a compound of formula 5-5; or

A compound of formula 2-9 is condensed with a compound of formula 5-6a or 5-6b under alkaline conditions to obtain a compound of formula 5-7, the compound of formula 5-7 is condensed with a compound of formula 5-8a or 5-8b under alkaline conditions to obtain a compound of formula 5-9, and the compound of formula 5-9 reacts with trifluoroethanol under the action of a condensing agent to obtain a compound of formula 5-10.

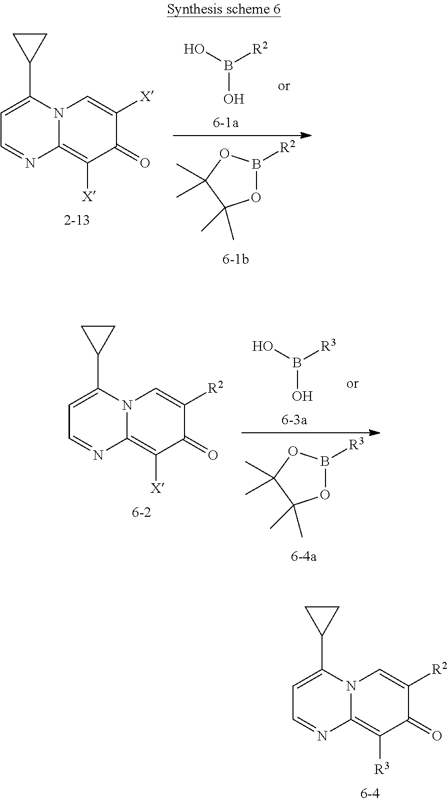

wherein X′ is selected from chlorine, bromine, or iodine; and R² and R³ are defined as described in formula I above.

Specifically, a compound of formula 2-13 is condensed with a compound of formula 6-1a or 6-1b under alkaline conditions to obtain a compound of formula 6-2, and the compound of formula 6-2 is condensed with a compound of formula 6-3a or 6-3b under alkaline conditions to obtain a compound of formula 6-4.

### BRIEF DESCRIPTION OF THE DRAWINGS

FIG. 1 is growth curve diagram of tumor volume in each group of Test Example 1;

FIG. 2 shows SAM test results in each group of tumors in Test Example 1.

### RELATED DEFINITIONS

Unless otherwise specified, the following terms used in the specification and claims have the following meanings:

The “compound” of the present invention may be asymmetric, for example, have one or more chiral centers. Unless otherwise specified, the “compound” of the present invention refers to any stereoisomer or a mixture of two or more stereoisomers. The stereoisomers include but are not limited to enantiomers and diastereoisomers. The compound containing asymmetric carbon atoms in the present invention may be separated in an optically active pure form or a form of a mixture of two or more stereoisomers. The optically active pure form may be split from a mixture of two or more stereoisomers, or synthesized by chiral raw materials or reagents.

The “compound” of the present invention further includes a tautomeric form. The tautomeric form originates from exchange of a single bond with adjacent double bonds accompanied by migration of a proton. For example,

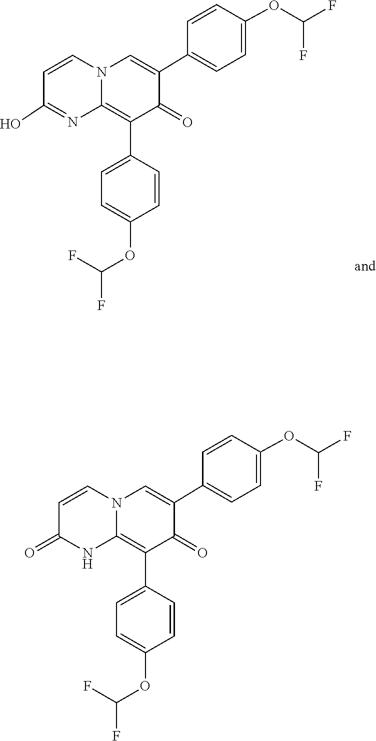 [SMILES: O=c1c(-c2ccc(OC(F)F)cc2)cn2ccc(O)nc2c1-c1ccc(OC(F)F)cc1 ; O=c1ccn2cc(-c3ccc(OC(F)F)cc3)c(=O)c(-c3ccc(OC(F)F)cc3)c2[nH]1]

are tautomerics of each other, and they can transform under certain conditions.

The term “optional” or “optionally” means that a subsequently described event or situation may or may not occur, including the occurrence and non-occurrence of the event or situation.

A numerical range herein refers to each integer in a given range. For example, “C1-C6” refers to a group may have 1 carbon atom, 2 carbon atoms, 3 carbon atoms, 4 carbon atoms, 5 carbon atoms, or 6 carbon atoms; and “C3-C6” refers to a group may have 3 carbon atoms, 4 carbon atoms, 5 carbon atoms, or 6 carbon atoms.

The term “membered” refers to the number of skeletal atoms or atomic groups that make up a ring. For example, “5-7-membered” refers to the number of skeletal atoms or atomic groups that make up a ring is 5, 6, or 7. Therefore, for example, pyridine, piperidine, piperazine, and benzene are six-membered ring, while thiophene and pyrrole are five-membered ring.

The term “substituted” refers to any one or more hydrogen atoms on a specific group are substituted by a substituent(s), as long as the valence state of the specific group is normal and the substituted compound is stable. For example, “substituted by halogen” refers to any one or more hydrogen atoms on a specific group are substituted by halogen, as long as the valence state of the specific group is normal and the substituted compound is stable.

The term “alkyl” refers to a saturated aliphatic hydrocarbon group, including straight or branched saturated hydrocarbon groups with indicated number of carbon atoms. For example, the term “C1-C6 alkyl” includes C1 alkyl, C2 alkyl, C3 alkyl, C4 alkyl, C5 alkyl, and C6 alkyl, and examples include, but are not limited to, methyl, ethyl, n-propyl, isopropyl, n-butyl, isobutyl, tert-butyl, n-pentyl, 2-pentyl, 3-pentyl, n-hexyl, 2-hexyl, 3-hexyl, and the like.

The term “alkoxy” refers to a group having an “alkyl-O—” structure, the alkyl is a straight or branched saturated monovalent hydrocarbon group. For example, “C1-C3 alkoxy” includes methoxy, ethoxy, n-propoxy, and isopropoxy.

The term “cycloalkyl” refers to a monocyclic saturated hydrocarbon system without heteroatoms and double bonds. Examples of the term “3-6-membered cycloalkyl” include, but are not limited to, cyclopropyl, cyclobutyl, cyclopentyl, and cyclohexyl.

The term “halogen” refers to fluorine, chlorine, bromine, and iodine.

The term “aryl” refers to an all-carbon monocylic or fused bicyclic aromatic ring group having a conjugated π electron system, which is obtained by removing one hydrogen atom from a single carbon atom of the parent aromatic ring system. The aryl includes bicyclic groups fused with saturated rings, partially unsaturated rings, or aromatic carbon rings. Examples include, but are not limited to, phenyl, naphthyl, anthracenyl, indene, indan, 1,2-dihydronaphthalene, and 1,2,3,4-tetrahydronaphthalene.

The term “heteroaryl” refers to a monovalent aryl including at least one heteroatom independently selected from nitrogen, oxygen, and sulfur. For example, examples of “5-7-membered heteroaryl” include, but are not limited to, pyridinyl, thiophenyl, imidazolyl, pyrimidinyl, pyridinyl, furyl, pyrazinyl, and thiazolyl.

The term “9-18-membered benzoheterocyclyl” refers to a ring system having 9-18 ring atoms or ring atomic groups fused by benzene ring and heterocyclic ring, the benzene ring and the heterocyclic ring share a pair of adjacent ring atoms, and a site bound to a parent nucleus structure is located in the benzene ring. The heterocyclic ring is a 5-12-membered saturated, partially unsaturated, or completely unsaturated ring system with ring carbon atoms and 1 to 4 ring heteroatoms or heteroatomic groups, and the heteroatoms or heteroatomic groups are independently selected from nitrogen, sulfur, oxygen, sulfoxide, sulfone,

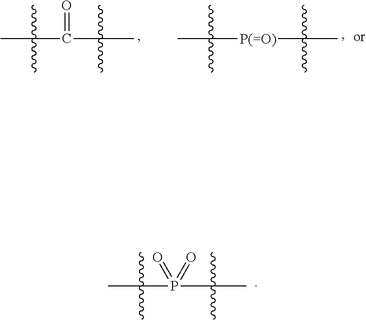

The heterocyclic ring may be a monocyclic, bicyclic or tricyclic systems, where two or more rings exist in the form of fused rings, spiro rings, or bridge rings. Examples include, but are not limited to

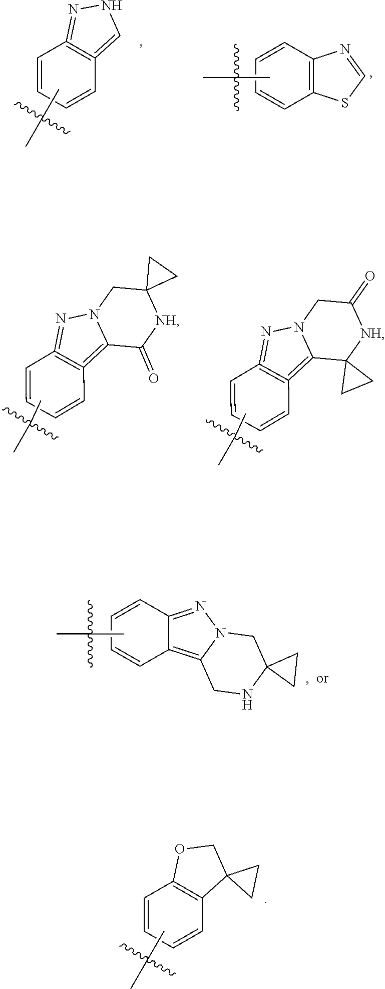

The “ [symbol] ” in

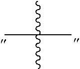

to the site where the chemical bond connects. When

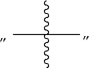

appears in a bicyclic or polycyclic ring and the connection position is uncertain, the binding site is any atom on the monocyclic ring where

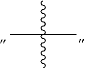

is located, as long as the atomic valence allows. For example,

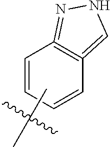

indicates that the connecting site is located on any carbon atom of the benzene ring in the bicyclic ring and needs to meet the requirements of atomic valence bonds.

The term “pharmaceutically acceptable salt” refers to a salt that retains the biological efficacy of free acids and bases of a specific compound without any biological adverse effects, such as acid (including organic and inorganic acids) addition salts or base addition salts (including organic and inorganic bases).

The pharmaceutically acceptable salt of the present invention may be synthesized by a conventional chemical method from a parent compound containing acid or base groups. Generally, a preparation method for such salts includes reaction of these compounds in a form of free acids or bases with stoichiometric appropriate bases or acids in water, organic solvents, or a mixture of both.

The term “effective amount” or “therapeutically effective amount” refers to an adequate dosage of a drug or agent that is non-toxic but can achieve desired effects. A precise dosage varies based on many factors, such as subject dependent variables (such as age and immune system health, etc.), disease or condition, and administered treatment.

The term “pharmaceutically acceptable carrier” refers to those carriers that have no obvious irritating effects on the body and do not impair the biological activity and performance of an active compound, including but not limited to any diluents, disintegrating agents, adhesives, flow aids, wetting agents that can be used for humans or animals as permitted by the State Food and Drug Administration.

The abbreviations used in the claims and specification have the following meanings:

M: mol/L; mM: mmol/L; μM: μmol/L; nM: nmol/L; TBAF: tetrabutylammonium fluoride; Brij35: polyoxyethylene lauryl ether; BSA: bovine serum albumin; DMSO: dimethyl sulfoxide; rpm: revolutions/minute; Tris-HCl: trihydroxymethyl aminomethane hydrochloride;

The term “pharmaceutical composition” refers to a composition including the compound or pharmaceutically acceptable salt thereof as disclosed in the present disclosure, as well as at least one of the following pharmaceutically acceptable ingredient selected based on properties of an application method and a dosage form, including but not limited to: carriers, diluents, adjuvants, excipients, preservatives, fillers, disintegrants, wetting agents, emulsifiers, suspensions, sweeteners, correctives, fragrances, antibacterial agents, anti-fungal agents, lubricants, dispersants, temperature-sensitive materials, temperature regulators, adhesives, stabilizers, suspension aids, and the like.

The drug or pharmaceutical composition of the present disclosure may be applied orally, locally, parenterally or mucosally (for example, orally, by inhalation, or rectally) in dosage units containing conventional non-toxic pharmaceutically acceptable carriers. Usually, an oral route is preferred. The active reagent may be applied orally in a form of capsules, tablets, or the like (see Remington: The Science and Practice of Pharmacy, 20th Edition).

The drug or pharmaceutical composition of the present disclosure may be delivered parenterally, namely, through intravenous (i.v.), intraventricular (i.e.v.), subcutaneous (s.c.), intraperitoneal (i.p.), intramuscular (i.m.), subcutaneous (s.d.), or intradermal (i.d.) administration, by direct injection, such as rapid concentrated injection or continuous infusion. A formulations used for injection may be presented in a unit dosage form, such as in an ampoule or multi-dose container with added preservative. The composition may be in a shape of an excipient, in a form of suspension, solution, or lotion in oil or aqueous carrier, and may include preparation reagents such as anti-settling agent, stabilizer, and/or dispersant. Alternatively, the active ingredient may be reconstituted in a powder form with a suitable carrier (such as sterile and heatless raw water) before use.

The drug or pharmaceutical composition of the present disclosure may further be formulated for rectal administration, such as suppositories or retention enemas (for example, containing conventional suppository matrices such as cocoa butter or other glycerides).

The term “treating” includes inhibiting, alleviating, preventing, or eliminating one or more symptoms or side effects related to the treated disease, condition, or disorder.

The use of the term “reduce”, “inhibit”, “alleviate”, or “decrease” is relative to controls. Those skilled in the art will easily determine appropriate controls for each experiment. For example, a reduction reaction in subjects or cells treated with a compound is compared with a reaction in subjects or cells not treated with the compound.

### DETAILED DESCRIPTION

Preparation methods for compounds of the present invention will be described in more detail below, but these specific preparation methods do not constitute any limitation on the scope of the present invention. Moreover, reaction conditions such as reactants, solvents, bases, amount of compounds used, reaction temperature, and reaction time are not limited to the following examples.

The compounds of the present invention can also be conveniently prepared by optionally combining various synthesis methods described in the specification or known in the art, and such combinations can be easily performed by those skilled in the art.

### Example 1: 1,3,8-tris(4-(difluoromethoxy)phenyl)-2H-quinolizin-2-one

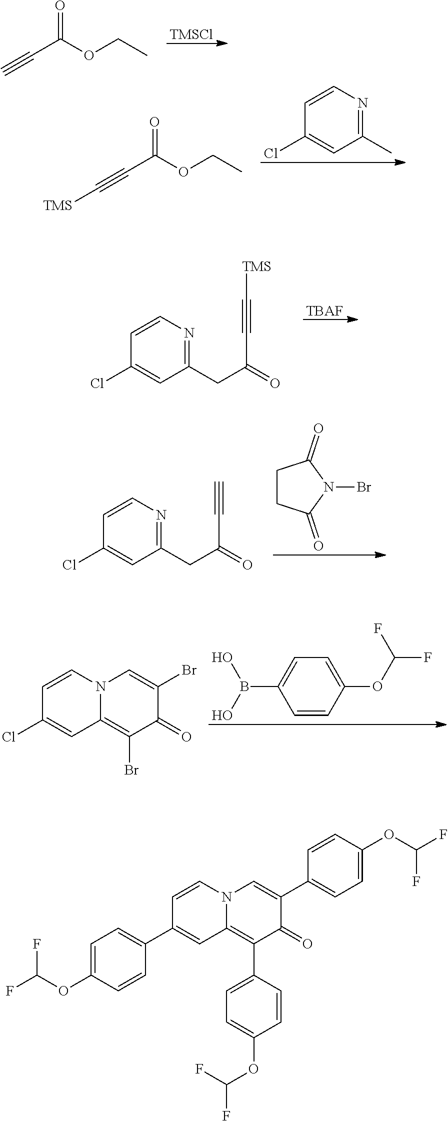

### a) Preparation of ethyl 3-(trimethylsilyl) propiolate

Ethyl propiolate (10 g), trimethylchlorosilane (14.40 g), and dichloromethane (200 ml) were sequentially added to a reaction flask under nitrogen protection, added with triethylamine (10.31 g) dropwise, and stirred at 40° C. to react for 3 h. After the reaction was completed, 100 ml of water was added for quenching, followed by extraction with 200 ml of ethyl acetate, concentration of an organic phase to dryness, and purification of a crude product by column chromatography (mobile phase: petroleum ether/ethyl acetate=8/1 (V/V)) to obtain 5.2 g of the title compound.

### b) Preparation of 1-(4-chloropyridin-2-yl)-4-(trimethylsilyl)-3-butyn-2-one

4-chloro-2-methylpyridine (5 g) and tetrahydrofuran (100 ml) were sequentially added to a reaction flask under nitrogen protection, cooled to −78° C. in a dry-ice ethanol bath, and added with lithium diisopropylamide (39.19 mL, 2M tetrahydrofuran solution) dropwise, and reacted at this temperature for 30 min. The ethyl 3-(trimethylsilyl) propiolate (7.01 g) was added for reaction at −78° C. for 1 h. After the reaction was completed, a saturated ammonium chloride solution (200 mL) was added for quenching, followed by three extractions with 100 ml of ethyl acetate for each, concentration of an organic phase to dryness, and purification of a crude product by column chromatography (mobile phase: petroleum ether/ethyl acetate=5/1 (V/V)) to obtain 5 g of the title compound. LCMS m/z=253.5 [M+1]⁺.

### c) Preparation of 1-(4-chloropyridin-2-yl)3-butyn-2-one

The 1-(4-chloropyridin-2-yl)-4-(trimethylsilyl)-3-butyn-2-one (1 g), tetrabutylammonium fluoride (1.56 ml, 1 M tetrahydrofuran solution), and tetrahydrofuran (20 ml) were sequentially added to a reaction flask and stirred at 0° C. to react for 30 min under nitrogen protection. After the reaction was completed, 10 ml of water was added for quenching, followed by extraction with 50 ml of ethyl acetate and concentration of an organic phase to dryness. A resulting crude product was directly used for the next step, and 0.6 g of the title compound was obtained. LCMS m/z=180.0[M+1]⁺.

### d) Preparation of 1,3-dibromo-8-chloro-2H-quinolizin-2-one

The 1-(4-chloropyridin-2-yl)3-butyn-2-one (0.3 g), dichloromethane (100 ml), and N-bromosuccinimide (0.89 g) were sequentially added to a reaction flask and reacted at room temperature for 2 h under nitrogen protection. After the reaction was completed, a saturated sodium thiosulfate solution (30 mL) was added for quenching, followed by three extractions with 30 ml of ethyl acetate for each and concentration of an organic phase to dryness. A resulting crude product was directly used for the next step, and 0.5 g of the title compound was obtained.

### e) Preparation of 1,3,8-tris(4-(difluoromethoxy)phenyl)-2H-quinolizin-2-one

The 1,3-dibromo-8-chloro-2H-quinolizin-2-one (1.3 g), 4-difluoromethoxy)phenyl boronic acid (2.17 g), potassium carbonate (1.6 g), [1,1′-bis(di-tert-butylphosphine) ferrocene]palladium dichloride (0.25 g), dioxane (26 ml), and water (5.2 ml) were sequentially added to a reaction flask and stirred at 80° C. to react for 1 h under nitrogen protection. After the reaction was completed, 30 ml of water was added for quenching, followed by extraction with 60 ml of ethyl acetate, concentration of an organic phase to dryness, and purification of a crude product by column chromatography (mobile phase: dichloromethane/methanol=40/1 (V/V)) to obtain 1,3,8-tris(4-(difluoromethoxy)phenyl)-2H-quinolizin-2-one and 8-chloro-1,3-bis(4-(difluoromethoxy)phenyl)-2H-quinolizin-2-one.

Structure confirmation data of 1,3,8-tris (4-(difluoromethoxy)phenyl)-2H-quinolizin-2-one ¹H NMR (400 MHz, DMSO-d6) δ 8.70 (s, 1H), 8.34 (d, J=7.4 Hz, 1H), 7.90-7.82 (m, 2H), 7.69-7.62 (m, 2H), 7.47-7.39 (m, 2H), 7.29 (m, 8H), 7.26 (d, J=2.3 Hz, 1H), 7.22-7.10 (m, 2H). LCMS m/z=572 [M+1]⁺.

### Example 2: 8-chloro-1,3-bis(4-(difluoromethoxy)phenyl)-2H-quinolizin-2-one

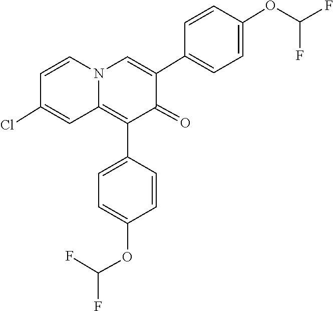 [SMILES: O=c1c(-c2ccc(OC(F)F)cc2)cn2ccc(Cl)cc2c1-c1ccc(OC(F)F)cc1]

Reference was made to the method of Example 1. Structure confirmation data of 8-chloro-1,3-bis(4-(difluoromethoxy)phenyl)-2H-quinolizin-2-one: ¹H NMR (400 MHz, DMSO-d6) δ 8.67 (s, 1H), 8.29-8.23 (m, 1H), 7.85-7.79 (m, 2H), 7.55-7.13 (m, 8H), 6.89-6.83 (m, 2H). LCMS m/z=464 [M+1]⁺.

### Example 3: 8-cyclopropyl-1,3-bis(4-(difluoromethoxy)phenyl)-2H-quinolizin-2-one

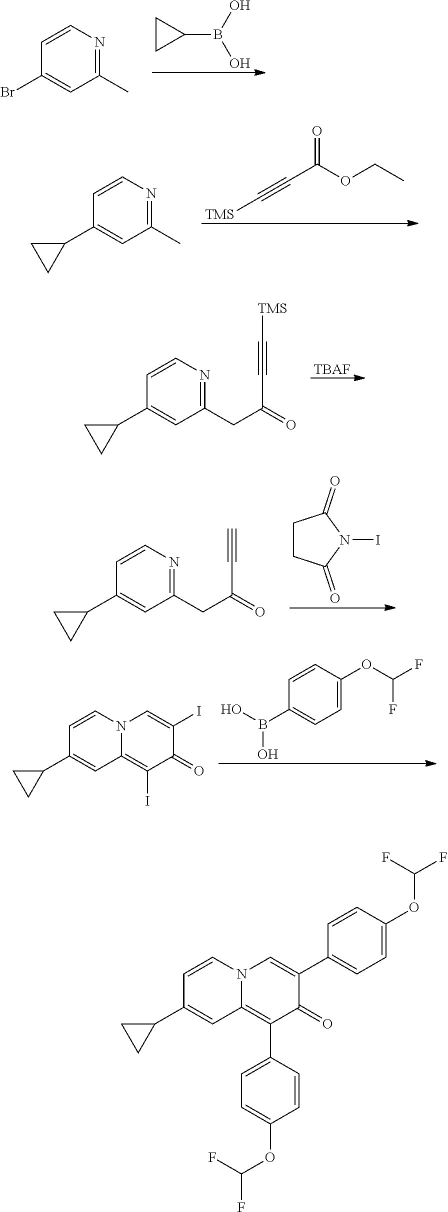

### a) Preparation of 4-cyclopropyl-2-methylpyridine

4-bromo-2-methylpyridine (10 g), cyclopropyl boronic acid (14.98 g), potassium phosphate (49.36 g), [1,1′-bis(di-tert-butylphosphine) ferrocene]palladium dichloride (0.25 g), water (40 ml), and dioxane (200 ml) were sequentially added to a reaction flask and stirred at 80° C. to react for 2 h under nitrogen protection. After the reaction was completed, 200 ml of water was added for quenching, followed by three extractions with 100 ml of ethyl acetate for each, concentration of an organic phase to dryness, and purification of a crude product by column chromatography (mobile phase: petroleum ether/ethyl acetate=9/1 (V/V)) to obtain 7.5 g of the title compound. LCMS m/z=134 [M+1]⁺.

### b) Preparation of 1-(4-cyclopropylpyridin-2-yl)-4-(trimethylsilyl)-3-butyn-2-one

With reference to the preparation method of Example 1, the 4-chloro-2-methylpyridine in step b) was replaced with 4-cyclopropyl-2-methylpyridine.

LCMS m/z=258 [M+1]⁺.

### c) Preparation of 1-(4-cyclopropylpyridin-2-yl)-3-butyn-2-one

With reference to the preparation method of Example 1/2, the 1-(4-chloropyrid-2-yl)-4-(trimethylsilyl)-3-butyl-2-ketone in step c) was replaced with 1-(4-cyclopropylpyridin-2-yl)-4-(trimethylsilyl)-3-butyn-2-one. LCMS m/z=185.95[M+1]⁺.

### d) Preparation of 8-cyclopropyl-1,3-diiodo-2-H-quinolizin-2-one

The 1-(4-cyclopropylpyridin-2-yl)-3-butyn-2-one (0.6 g), dichloromethane (12 ml), and N-iodosuccinimide (1.82 g) were sequentially added to a reaction flask and reacted at 0° C. for 1 h under nitrogen protection. After the reaction was completed, a saturated sodium thiosulfate solution (30 mL) was added for quenching, followed by three extractions with 30 ml of ethyl acetate for each, concentration of an organic phase to dryness, and purification of a crude product by column chromatography (mobile phase: petroleum ether/ethyl acetate=1/1 (V/V)) to obtain 0.3 g of the title compound. LCMS m/z=437.8 [M+1]⁺.

### e) Preparation of 8-cyclopropyl-1,3-bis(4-(difluoromethoxy)phenyl)-2H-quinolizin-2-one

With reference to the preparation method of Example 1, the 1,3-dibromo-8-chloro-2H-quinolizin-2-one in step e) was replaced with 8-cyclopropyl-1,3-diiodo-2-H-quinolizin-2-one.

¹H NMR (400 MHz, DMSO-d6) δ 8.57 (s, 1H), 8.14 (d, J=7.4 Hz, 1H), 7.87-7.78 (m, 2H), 7.49-7.13 (m, 4H), 7.26 (t, J=11.4 Hz, 4H), 6.72 (d, J=2.1 Hz, 1H), 6.39 (dd, J=7.4, 2.0 Hz, 1H), 1.84 (tt, J=8.4, 4.8 Hz, 1H), 1.02-0.92 (m, 2H), 0.78-0.70 (m, 2H). LCMS m/z=470 [M+1]⁺.

### Example 4: 8-ethyl-1,3-bis(4-(difluoromethoxy)phenyl)-2H-quinolizin-2-one

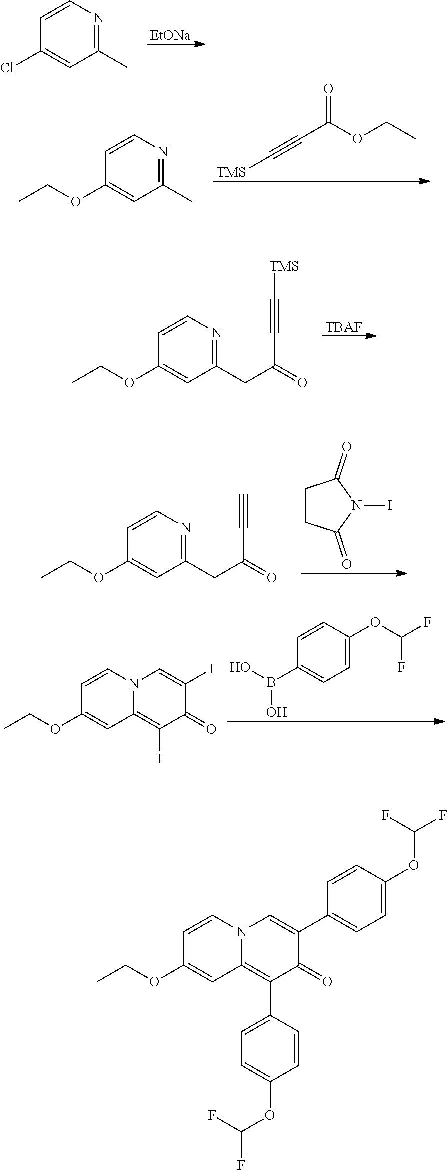

### a) Preparation of 4-ethoxy-2-methylpyridine

4-chloro-2-methylpyridine (5 g), sodium ethanol (13.34 g), and dimethyl sulfoxide (100 ml) were sequentially added to a reaction flask and stirred at 50° C. to react for 2 h under nitrogen protection. After the reaction was completed, 200 ml of water was added for quenching, followed by three extractions with 100 ml of ethyl acetate for each and concentration of an organic phase to dryness, to obtain 4.6 g of the title compound.

### b) Preparation of 1-(4-ethoxypyridin-2-yl)-4-(trimethylsilyl)-3-butyn-2-one

With reference to the preparation method of Example 1/2, the 4-chloro-2-methylpyridine in step b) was replaced with 4-ethyl-2-methylpyridine. LCMS m/z=262 [M+1]⁺.

### c) Preparation of 1-(4-ethoxypyridin-2-yl)3-butyn-2-one

With reference to the preparation method of Example 1/2, the 1-(4-chloropyridin-2-yl)-4-(trimethylsilyl)-3-butyl-2-one in step c) was replaced with 1-(4-ethoxypyridin-2-yl)-4-(trimethylsilyl)-3-butyn-2-one. LCMS m/z=189.90 [M+1]⁺.

### d) Preparation of 8-ethoxy-1,3-diiodo-2-H-quinolizin-2-one

With reference to the preparation method of Example 3, the 1-(4-cyclopropylpyridin-2-yl)3-butyn-2-one in step d) was replaced with the 1-(4-ethoxypyridin-2-yl)3-butyn-2-one. LCMS m/z=441.8 [M+1]⁺.

### e) Preparation of 1,3-bis(4-(difluoromethoxy)phenyl)-8-ethoxy-2H-quinolizin-2-one

With reference to the preparation method of Example 1/2, the 1,3-dibromo-8-chloro-2H-quinolizin-2-one in step e) was replaced with the 8-ethoxy-1,3-diiodo-2-H-quinolizin-2-one.

¹H NMR (400 MHz, DMSO-d6) δ 8.52 (s, 1H), 8.18 (d, J=7.7 Hz, 1H), 7.85-7.76 (m, 2H), 7.41-7.33 (m, 2H), 7.49-7.12 (t, J=72 Hz, 2H), 7.30-7.20 (m, 4H), 6.58 (dd, J=7.7, 2.7 Hz, 1H), 6.12 (d, J=2.7 Hz, 1H), 3.88 (q, J=6.9 Hz, 2H), 1.26 (q, J=5.9, 4.9 Hz, 3H). LCMS m/z=474 [M+1]⁺.

### Example 5: 1,3-bis(3-amino-2-methyl-2H-indazol-5-yl)-8-cyclopropylquinolizin-2-one

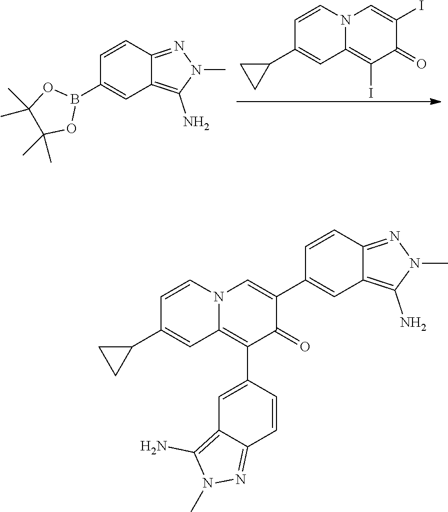

### a) Preparation of 1,3-bis(3-amino-2-methyl-2H-indazol-5-yl)-8-cyclopropylquinolizin-2-one

2-methyl-5-(4,4,5,5-tetramethyl-1,3,2-dioxaborolan-2-yl) indole-3-amine (180 mg), 1,4-dioxane (3.60 mL), water (0.80 mL), 8-cyclopropyl-1,3-diiodoquinolizin-2-one (86.40 mg), potassium carbonate (273.23 mg), and [1,1′-bis(diphenylphosphino)ferrocene]palladium(II)dichloride dichloromethane complex (53.68 mg) were added to a reaction flask and stirred at 30° C. for 48 h under nitrogen protection, and the reaction solution was concentrated under reduced pressure to prepare and separate 4.7 mg of the title compound. Preparation and separation conditions: (chromatographic column: YMC-Actus Triart C18, 30*150 mm, 5 μm; mobile phase A: water (10 mmol/L ammonium formate), mobile phase B: acetonitrile; flow rate: 60 mL/min; gradient: 30% B to 60% B in 8 min, 60% B; test wavelength: 220 nm; retention time (min): 7.93; column temperature: 25° C.).

¹H NMR (400 MHz, DMSO-d₆) δ 8.46 (s, 1H), 8.16 (d, J=7.5 Hz, 2H), 7.51 (s, 1H), 7.37 (dd, J=9.0, 1.7 Hz, 1H), 7.19 (dd, J=16.4, 8.9 Hz, 2H), 6.92 (dd, J=8.9, 1.6 Hz, 1H), 6.78 (d, J=2.0 Hz, 1H), 6.30 (dd, J=7.3, 2.0 Hz, 1H), 6.14 (s, 2H), 6.03 (s, 2H), 3.79 (d, J=10.9 Hz, 6H), 1.77 (tt, J=8.5, 4.9 Hz, 1H), 0.95-0.88 (m, 2H), 0.70 (t, J=2.8 Hz, 2H). LCMS m/z=476 [M+1]⁺.

### Example 6: 3-(3-amino-2-methyl-2H-indazol-5-yl)-8-cyclopropyl-1-(2,2-difluoro-1,3-benzodioxol-5-yl) quinolizin-2-one hydrochloride

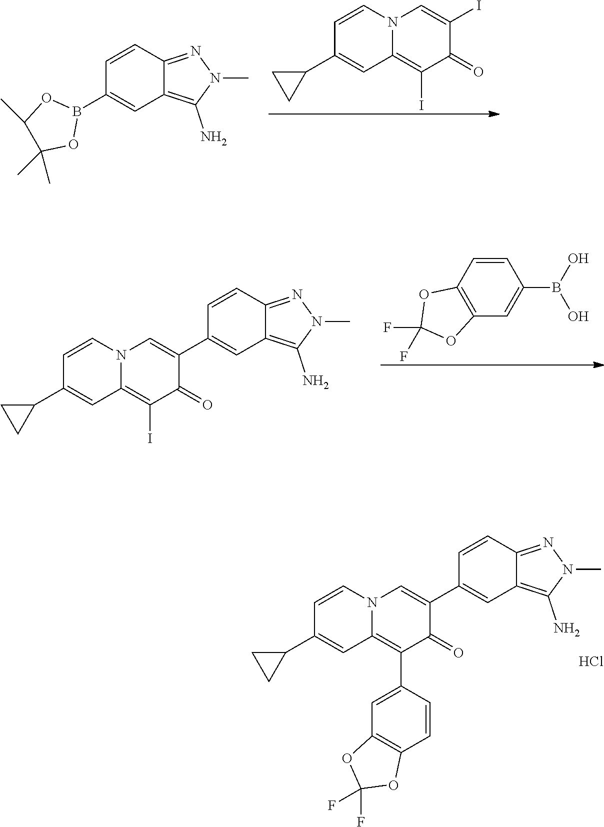

### a) Preparation of 3-(3-amino-2-methyl-2H-indazol-5-yl)-8-cyclopropyl-1-iodoquinolizin-2-one

Reference was made to the preparation method of Example 5.

### b) Preparation of 3-(3-amino-2-methyl-2H-indazol-5-yl)-8-cyclopropyl-1-(2,2-difluoro-1,3-benzodioxol-5-yl) quinolizin-2-one hydrochloride

2,2-difluoro-1,3-benzodioxol-5-yl boronic acid (10.62 mg), 1,4-dioxane (0.5 mL), water (0.1 mL), the 3-(3-amino-2-methyl-2H-indazol-5-yl)-8-cyclopropyl-1-iodoquinolizin-2-one (16 mg), potassium carbonate (14.54 mg), and [1,1′-bis(diphenylphosphino)ferrocene]palladium(II)dichloride dichloromethane complex (2.86 mg) were added to a reaction flask and stirred at 80° C. for 1 h under nitrogen protection, and the reaction solution was concentrated under reduced pressure to prepare and separate 2.6 mg of the title compound. Preparation and separation conditions: (chromatographic column: YMC-Actus Triart C18, 30*150 mm, 5 μm; mobile phase A: water (10 mmol/L ammonium formate), mobile phase B: acetonitrile; flow rate: 60 mL/min; gradient: 15% B to 44% B in 8 min, 44% B; test wavelength: 220 nm; retention time (min): 7.32; column temperature: 25° C.).

¹H NMR (400 MHz, DMSO-d₆) δ 8.70 (d, J=4.1 Hz, 1H), 8.51 (d, J=4.4 Hz, 1H), 8.29 (s, 1H), 7.90 (d, J=8.8 Hz, 1H), 7.52 (dd, J=8.5, 6.4 Hz, 2H), 7.35 (d, J=1.6 Hz, 1H), 7.12 (dd, J=8.2, 1.5 Hz, 1H), 6.88 (s, 1H), 6.53 (s, 1H), 3.83 (s, 3H), 1.96 (d, J=9.5 Hz, 1H), 1.08-0.97 (m, 2H), 0.88-0.76 (m, 2H). LCMS m/z=487 [M+1]⁺.

### Example 7: 8-cyclopropyl-1-(2,2-difluoro-1,3-benzodioxol-5-yl)-3-[2-methyl-3-(2-methylpyrazol-3-yl) indazol-5-yl]quinolizin-2-one

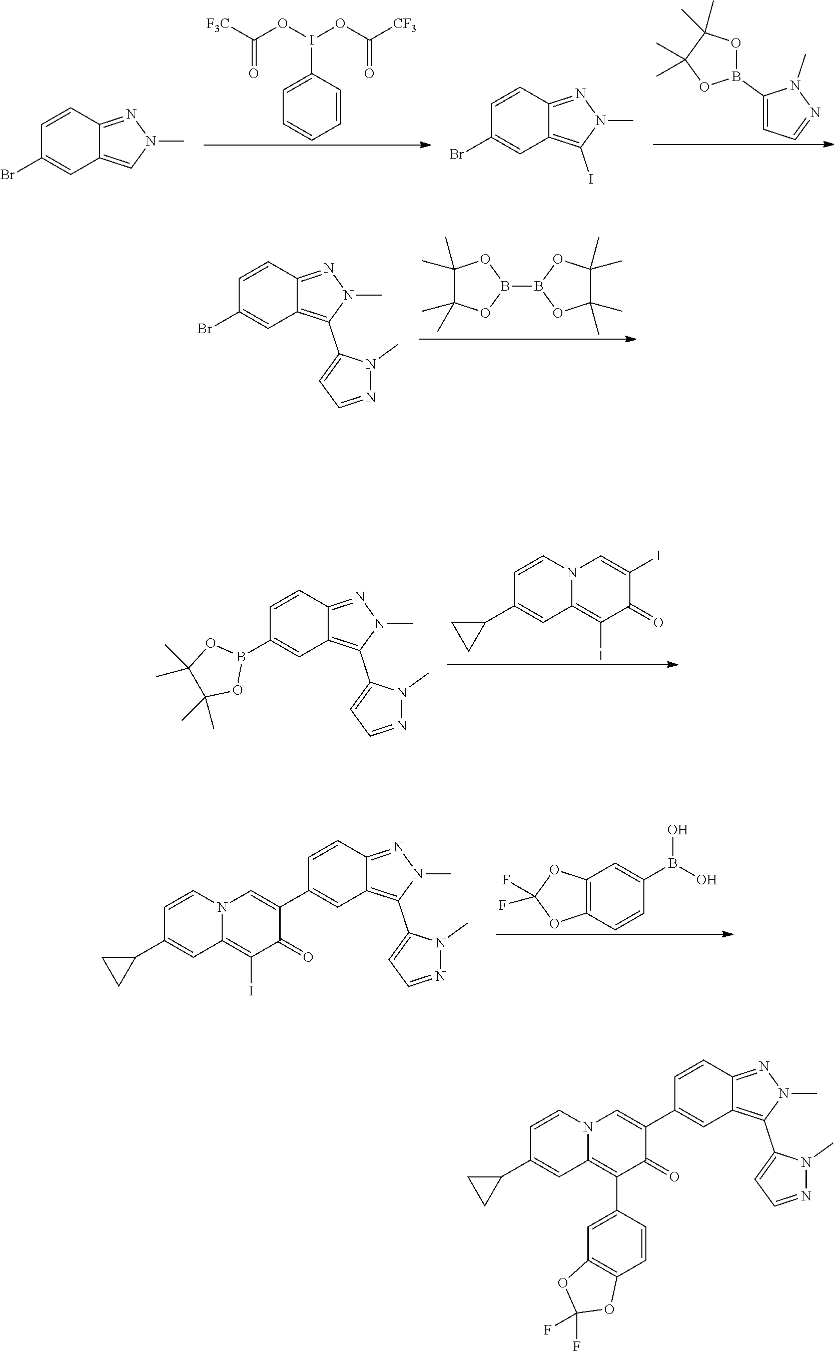

### a) Preparation of 5-bromo-3-iodo-2-methylindazole

5-bromo-2-methylindazole (5 g), pyrazine (2.9 g), and dichloromethane (50 mL) were added to a reaction flask, added with phenyl-3-iododibis(2,2,2-trifluoroacetate) (12.2 g) while stirring, stirred at 30° C. for 30 min under nitrogen protection, added with iodine (7.22 g), and continuously stirred for 24 hours, and the reaction solution was washed with dichloromethane (2×100 mL) to obtain 4.1 g of the title compound.

### b) Preparation of 5-bromo-2-methyl-3-(2-methylpyrazol-3-yl)indazole

The 5-bromo-3-iodo-2-methylindazole (1 g), 1-methyl-5-(4,4,5,5-tetramethyl-1,3,2-dioxaborolan-2-yl) pyrazole (1.54 g), 1,4-dioxane/water=5:1 (20 ml), [1,1′-bis(diphenylphosphino)ferrocene]palladium(II)dichloride dichloromethane complex (0.73 g), and potassium phosphate (4.41 g) were added to a reaction flask and stirred at 80° C. for 24 hours under nitrogen protection, and the reaction was quenched with water at room temperature, followed by extraction with ethyl acetate (2×20 mL), reduced pressure concentration, and purification by column chromatography (mobile phase: ethyl acetate/petroleum ether=3/10 (V/V)), to obtain 290 mg of the title compound.

### c) Preparation of 2-methyl-3-(2-methylpyrazol-3-yl)-5-(4,4,5,5-tetramethyl-1,3,2-dioxaborolan-2-yl) indazole

The 5-bromo-2-methyl-3-(2-methylpyrazol-3-yl)indazole (200 mg), bis(pinacolato)diboron (261.7 mg), and 1,4-dioxane (20 mL) were added to a reaction flask, then added with [1,1′-bis(diphenylphosphino)ferrocene]palladium(II)dichloride dichloromethane complex (5 mg) and potassium acetate (200 mg) under nitrogen protection, and stirred at 80° C. for 2 hours, the reaction solution was concentrated under reduced pressure, and the concentrate was purified by column chromatography (mobile phase: ethyl acetate/petroleum ether=3/10 (V/V))) to obtain 130 mg of the title compound.

### d) Preparation of 8-cyclopropyl-1-iodo-3-[2-methyl-3-(2-methylpyrazol-3-yl)indazol-5-yl]quinolizin-2-one

With reference to the preparation method of Example 6, the 2-methyl-5-(4,4,5,5-tetramethyl-1,3,2-dioxaborolan-2-yl)indazole-3-amine in step a) was replaced with the 2-methyl-3-(2-methylpyrazol-3-yl)-5-(4,4,5,5-tetramethyl-1,3,2-dioxaborolan-2-yl)indazole.

### e) Preparation of 8-cyclopropyl-1-(2,2-difluoro-1,3-benzodioxol-5-yl)-3-[2-methyl-3-(2-methylpyrazol-3-yl) indazol-5-yl]quinolizin-2-one

Reference was made to the preparation method of step b) in Example 6.

¹H NMR (400 MHz, DMSO-d₆) δ 8.62 (s, 1H), 8.17 (d, J=7.4 Hz, 1H), 8.01 (d, J=1.3 Hz, 1H), 7.70 (dd, J=6.5, 1.6 Hz, 3H), 7.46 (d, J=8.2 Hz, 1H), 7.32 (d, J=1.6 Hz, 1H), 7.08 (dd, J=8.3, 1.6 Hz, 1H), 6.74 (dd, J=24.9, 1.9 Hz, 2H), 6.37 (dd, J=7.4, 2.0 Hz, 1H), 4.08 (s, 3H), 3.75 (s, 3H), 1.94-1.85 (m, 1H), 0.96 (dd, J=8.2, 2.4 Hz, 2H), 0.81-0.69 (m, 2H). LCMS m/z=552[M+1]⁺.

### Example 8: 8-cyclopropyl-1,3-bis[2-methyl-3-(2-methylpyrazol-3-yl)indazol-5-yl]quinolizin-2-one

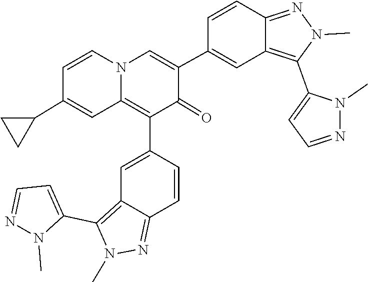 [SMILES: Cn1nccc1-c1c2cc(-c3cn4ccc(C5CC5)cc4c(-c4ccc5nn(C)c(-c6ccnn6C)c5c4)c3=O)ccc2nn1C]

Reference was made to the preparation method of Example 7.

¹H NMR (400 MHz, DMSO-d₆) δ 8.59 (s, 1H), 8.14 (d, J=7.4 Hz, 1H), 7.98 (t, J=1.3 Hz, 1H), 7.78-7.61 (m, 5H), 7.36 (t, J=1.2 Hz, 1H), 7.22 (dd, J=8.9, 1.5 Hz, 1H), 6.81 (d, J=2.0 Hz, 1H), 6.70 (dd, J=3.9, 1.9 Hz, 2H), 6.36 (dd, J=7.4, 2.0 Hz, 1H), 4.08 (d, J=8.9 Hz, 6H), 3.75 (d, J=4.0 Hz, 6H), 1.78 (tt, J=8.5, 4.9 Hz, 1H), 0.99-0.86 (m, 2H), 0.76-0.62 (m, 2H). LCMS m/z=606[M+1]⁺.

### Example 9: 9-[4-(difluoromethoxy)phenyl]-7-(2-methyl-2H-indazol-5-yl)-2-[(2,2,2-trifluoroethyl)amino]-8H-pyrido[1,2-a]pyrimidin-8-one

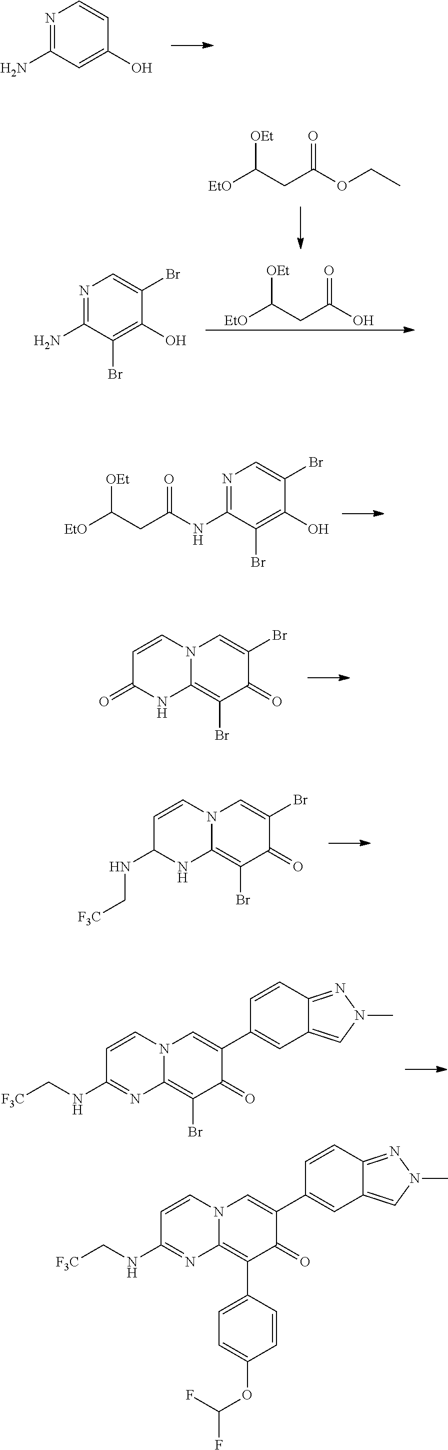

### a) Preparation of 3,3-diethoxypropanoic acid

Ethyl 3,3-Diethoxypropionate (20 g) was gradually added to a water (30 mL) solution of sodium hydroxide (5.47 g), and the solution was stirred at 100° C. for 1 h under nitrogen displacement protection and cooled to room temperature. The pH was adjusted to 6 with 3M diluted hydrochloric acid at 0° C., and the solution was extracted with ethyl acetate (6×30 mL). The organic phase was washed with saturated salt solution (1×30 mL), followed by drying with anhydrous sodium sulfate, filtration, and reduced pressure concentration to obtain 16.7 g of the title compound.

### b) Preparation of 2-amino-3,5-dibromopyridin-4-ol

2-aminopyridin-4-ol (10 g), N,N-dimethylformamide (150 mL), and N-bromosuccinimide (35.56 g) were sequentially added to a reaction flask and stirred at room temperature for 4 h under nitrogen displacement protection, the solution was filtered to collect a settling solid, and the solid was washed with acetonitrile (3×30 mL) to obtain 14 g of the title compound.

### c) Preparation of N-(3,5-dibromo-4-hydroxypyridin-2-yl)-3,3-diethoxypropanamide

Under nitrogen protection, the 3,3-diethoxypropionic acid (12.11 g), 1-hydroxybenzotriazole (12.61 g), dicyclohexylcarbodiimide (19.25 g), and N,N-dimethylformamide (200 mL) were sequentially added to a reaction flask, stirred at room temperature, then added with N,N-diisopropylethylamine (24.12 g), stirred for 5 minutes, added with the 2-amino-3.5-dibromopyridin-4-ol (10 g), stirred at 80° C. for 4 h, and cooled to room temperature. The obtained mixture was filtered, and the filter cake was washed with N,N-dimethylformamide (3×20 mL). The filtrate was concentrated under reduced pressure for sample preparation and purified by column chromatography (mobile phase: dichloromethane/methanol=15/1 (V/V)) to obtain 1.5 g of the title compound and 1.5 g of 7,9-dibromo-1H-pyrido[1,2-a]pyrimidin-2,8-dione.

### d) Preparation of 7,9-dibromo-1H-pyrido[1,2-a]pyrimidin-2,8-dione

The N-(3,5-dibromo-4-hydroxypyridin-2-yl)-3,3-diethoxypropanamide (2 g) was added to dilute hydrochloric acid (6 M, 60 mL), and the reaction solution was stirred at 50° C. for 12 hours under nitrogen displacement protection and concentrated under reduced pressure. acetonitrile (4 mL) was added to the crude product for pulping and purification, and the settling solid was filtered and collected to obtain 1 g of the title compound.

### e) Preparation of 7,9-dibromo-2-[(2,2,2-trifluoroethyl)amino]-8H-pyrido[1,2-a]pyrimidin-8-one

The 7,9-dibromo-1H-pyrido[1,2-a]pyrimidin-2,8-dione (500 mg), 1H-benzotriazol-1-yloxytripyrrolyl hexafluorophosphate (813.27 mg), 2,2,2-trifluoroethylamine (309.61 mg), N,N-dimethylformamide (7.5 mL), and N,N-diisopropylethylamine (403.96 mg) were sequentially added to a reaction flask and stirred at room temperature for 3 h under nitrogen displacement protection, the reaction was quenched with ice water (15 ml), the solution was filtered to collect settling solid, the solid was washed with acetonitrile (2×3 ml), and the obtained solid was dried in vacuum to obtain 400 mg of the title compound.

### f) Preparation of 9-bromo-7-(2-methyl-2H-indazol-5-yl)-2-[(2,2,2-trifluoroethyl)amino]-8H-pyrido[1,2-a]pyrimidin-8-one

The 7,9-dibromo-2-[(2,2,2-trifluoroethyl)amino]-8H-pyrido[1,2-a]pyrimidin-8-one (280 mg), 2-methyl-2H-indazol-5-yl boronic acid (245.77 mg), [1,1′-bis(diphenylphosphino)ferrocene]palladium(II)dichloride dichloromethane complex (56.98 mg), and potassium phosphate (444.67 mg) were sequentially added to a reaction flask, then added with 1,4-dioxane (6 mL) and water (1.2 mL), and stirred at 80° C. for 2 h under nitrogen displacement protection, the reaction solution was concentrated under reduced pressure to prepare sand, and the concentrate was purified by column chromatography (mobile phase: dichloromethane/methanol=10/1 (V/V)) to obtain 120 mg of the title compound.

### g) Preparation of 9-[4-(difluoromethoxy)phenyl]-7-(2-methyl-2H-indazol-5-yl)-2-[(2,2,2-trifluoroethyl)amino]-8H-pyrido[1,2-a]pyrimidin-8-one

The 9-bromo-7-(2-methyl-2H-indazol-5-yl)-2-[(2,2,2-trifluoroethyl)amino]-8H-pyrido[1,2-a]pyrimidin-8-one (40 mg), 4-(difluoromethoxy)phenyl boronic acid (33.25 mg), [1,1′-bis(diphenylphosphino)ferrocene]palladium(II)dichloride dichloromethane complex (7.21 mg), and potassium phosphate (56.32 mg) were sequentially added to a reaction flask and then added with 1,4-dioxane (1 mL) and water (0.2 mL), the reaction solution was stirred at 80° C. for 2 hours under nitrogen displacement protection and cooled to room temperature, and 10 mg of the title compound was prepared and separated. Preparation and separation conditions: (chromatographic column: XBridge Prep OBD C18, 30*150 mm, 5 μm; mobile phase A: water (10 mmol/L ammonium formate), mobile phase B: acetonitrile; flow rate: 60 mL/min; gradient: 25% B to 65% B in 8 min, 65% B; test wavelength: 220 nm; retention time (min): 7.22; column temperature: 25° C.).

¹H NMR (400 MHz, DMSO-d₆) δ 8.56 (s, 1H), 8.38 (s, 1H), 8.24 (d, J=8.2 Hz, 2H), 8.06 (s, 1H), 7.59 (d, J=9.1 Hz, 1H), 7.55-7.46 (m, 3H), 7.24 (t, J=49.6 Hz 1H), 7.15-7.03 (m, 2H), 6.35 (d, J=7.4 Hz, 1H), 4.18 (s, 3H), 4.05 (s, 2H). LCMS m/z=516 [M+1]⁺.

### Example 10: 7,9-bis[4-(difluoromethoxy)phenyl]-2-[(2,2,2-trifluoroethyl)amino]-8H-pyrido[1,2-a]pyrimidin-8-one

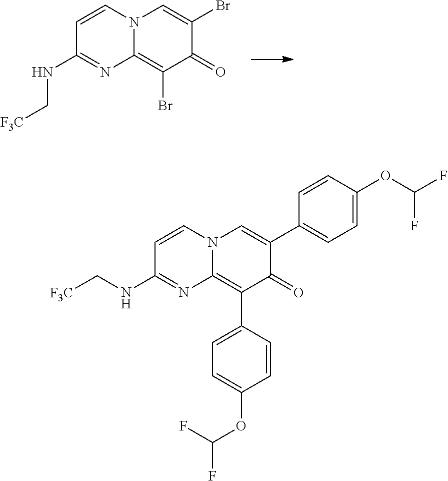 [SMILES: O=c1c(Br)cn2ccc(NCC(F)(F)F)nc2c1Br ; O=c1c(-c2ccc(OC(F)F)cc2)cn2ccc(NCC(F)(F)F)nc2c1-c1ccc(OC(F)F)cc1]

### a) Preparation of 7,9-bis[4-(difluoromethoxy)phenyl]-2-[(2,2,2-trifluoroethyl)amino]-8H-pyrido[1,2-a]pyrimidin-8-one

7,9-dibromo-2-[(2,2,2-trifluoroethyl)amino]-8H-pyrido[1,2-a]pyrimidin-8-one (20 mg), 4-(difluoromethoxy)phenyl boronic acid (70.31 mg), [1,1′-bis(diphenylphosphino)ferrocene]palladium(II)dichloride dichloromethane complex (10.16 mg), and potassium phosphate (79.41 mg) were sequentially added to a reaction flask and then added with 1,4-dioxane (2 mL) and water (0.5 mL), the reaction solution was stirred at 80° C. for 4 hours under nitrogen displacement protection and cooled to room temperature, and 10 mg of the title compound was prepared and separated. Preparation and separation conditions: (chromatographic column: XBridge Prep OBD C18, 30*150 mm, 5 μm; mobile phase A: water (10 mmol/L ammonium formate), mobile phase B: acetonitrile; flow rate: 60 mL/min; gradient: 25% B to 65% B in 8 min, 65% B; test wavelength: 220 nm; retention time (min): 7.83; column temperature: 25° C.).

¹H NMR (400 MHz, DMSO-d₆) δ 8.60 (s, 1H), 8.20 (t, J=3.9 Hz, 2H), 7.75 (d, J=8.5 Hz, 2H), 7.47 (t, J=7.4 Hz, 2H), 7.41-6.98 (m, 6H), 6.35 (d, J=7.5 Hz, 1H), 4.03 (d, J=10.0 Hz, 2H). LCMS m/z=528 [M+1]⁺.

### Example 11: 7,9-bis[(4-methoxyphenyl)]-2-[(2,2,2-trifluoroethyl)amino]-8H-pyrido[1,2-a]pyrimidin-8-one

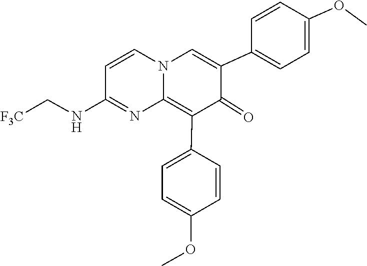 [SMILES: COc1ccc(-c2cn3ccc(NCC(F)(F)F)nc3c(-c3ccc(OC)cc3)c2=O)cc1]

With reference to the preparation method of Example 10, the 4-(difluoromethoxy)phenyl boronic acid in step a) was replaced with 4-methoxyphenyl boronic acid.

¹H NMR (400 MHz, DMSO-d₆) δ 8.48 (t, J=6.3 Hz, 1H), 8.19 (d, J=7.5 Hz, 1H), 8.11 (s, 1H), 7.64 (d, J=8.5 Hz, 2H), 7.37 (d, J=8.3 Hz, 2H), 6.97 (d, J=8.5 Hz, 2H), 6.84 (d, J=8.4 Hz, 2H), 6.30 (d, J=7.5 Hz, 1H), 4.05 (dd, J=9.9, 6.5 Hz, 2H), 3.78 (d, J=10.5 Hz, 6H). LCMS m/z=456 [M+1]⁺.

### Example 12: 7,9-bis[(2-methyl-2H-indazol-5-yl)]-2-[(2,2,2-trifluoroethyl)amino]-8H-pyrido[1,2-a]pyrimidin-8-one

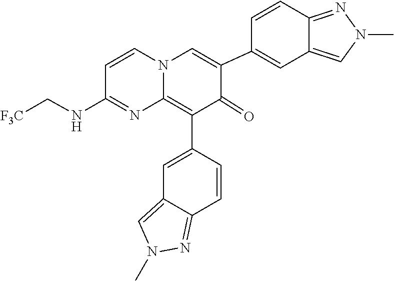 [SMILES: Cn1cc2cc(-c3cn4ccc(NCC(F)(F)F)nc4c(-c4ccc5nn(C)cc5c4)c3=O)ccc2n1]

With reference to the preparation method of Example 10, the 4-(difluoromethoxy)phenyl boronic acid in step a) was replaced with 2-methyl-2H-indazol-5-yl boronic acid.

¹H NMR (400 MHz, DMSO-d₆) δ 8.50 (t, J=6.3 Hz, 1H), 8.38 (s, 1H), 8.23 (dd, J=9.5, 7.2 Hz, 3H), 8.08 (t, J=1.3 Hz, 1H), 7.73 (s, 1H), 7.62-7.52 (m, 2H), 7.43 (d, J=9.0 Hz, 1H), 7.32 (dd, J=9.0, 1.6 Hz, 1H), 6.33 (d, J=7.5 Hz, 1H), 4.17 (d, J=8.5 Hz, 6H), 4.09-3.90 (m, 2H). LCMS m/z=504 [M+1]⁺.

### Example 13: 9-{6,7-dihydro-2H-spiro[1-benzofuran-3,1′-cyclopropane]-5-yl}-7-{2H spiro[1-benzofuran-3,1′-cyclopropane]-5-yl}-2-[(2,2,2-trifluoroethyl)amino]-8H-pyrido[1,2-a]pyrimidin-8-one

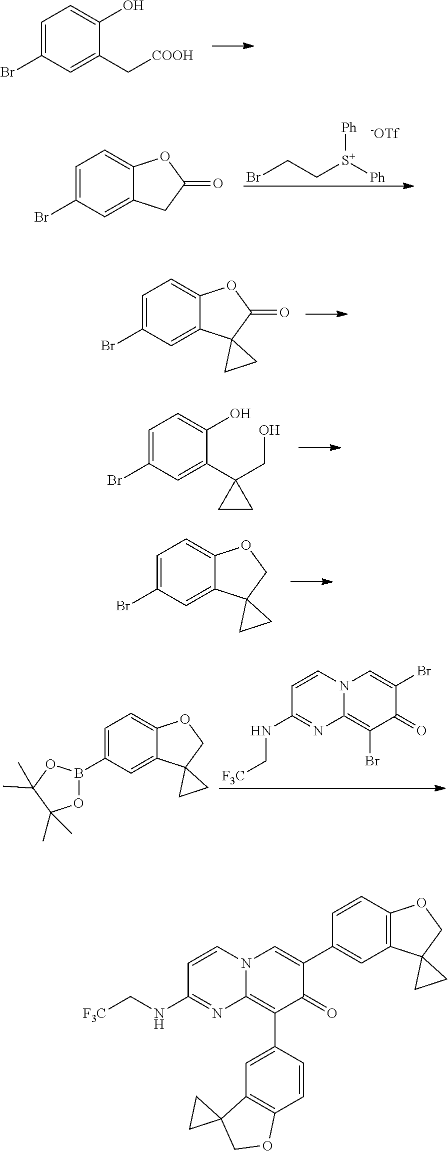

### a) Preparation of 5-bromobenzofuran-2(3H)-one

2-(5-bromo-2-hydroxyphenyl) acetic acid (5.8 g) and toluene (50 mL) were added to a flask, heated to 100° C., then added with 0.5 mL of concentrated sulfuric acid dropwise, heated and refluxed for 6 hours. After the reaction was completed, the reaction solution was cooled to room temperature, and then added with saturated sodium bicarbonate aqueous solution while stirring until no bubble was generated. An organic phase was separated and washed with saturated salt solution. The organic phase was concentrated to dryness to obtain 3.8 g of the title compound.

### b) Preparation of 5-bromo-2H-spiro[benzofuran-3,1′-cyclopropane]-2-one

The 5-bromobenzofuran-2(3H)-one (2.1 g), (2-bromoethyl)diphenylsulfonium trifluoromethane sulfonate (6.6 g), and N,N-dimethylformamide (50 mL) were added to a flask, stirred at room temperature for 5 minutes, then added with triethylamine (3.1 g), and continuously stirred at room temperature for 6 hours. After the reaction was completed, ethyl acetate (150 mL) was added, followed by washing with saturated salt solution (3*50 mL). An organic phase was concentrated to dryness, and a crude product was purified by column chromatography (mobile phase: petroleum ether/ethyl acetate=20/1 (V/V)) to obtain 1 g of the title compound.

### c) Preparation of 4-bromo-2-(1-(hydroxymethyl)cyclopropyl) phenol

The 5-bromo-2H-spiro[benzofuran-3,1′-cyclopropane]-2-one (880 mg) and tetrahydrofuran (20 mL) were added to a flask, protected by nitrogen, cooled to 0° C., then added with lithium aluminum hydride (70 mg), and continuously stirred for 1 hour. The reaction was monitored with a thin layer chromatography plate. If the reaction was incomplete, lithium aluminum hydride (35 mg) was added again, and the stirring continued for 1 hour until the raw materials reacted completely. After the reaction was completed, water (20 mL) was added, then hydrochloric acid aqueous solution (2 M, 5 mL) was added, and the stirring continued for ten minutes. Then the solution was extracted with ethyl acetate (3*20 mL), and an organic phase was collected, dried, and concentrated. A crude product was purified by column chromatography (mobile phase: petroleum ether/ethyl acetate=10/1 (V/V)) to obtain 820 mg of the title compound.

### d) Preparation of 5-bromo-2H-spiro[benzofuran-3,1′-cyclopropane]

The 4-bromo-2-(1-(hydroxymethyl)cyclopropyl) phenol (486 mg) and dimethyl carbonate (10 mL) were added to a flask and stirred, and then potassium tert-butoxide was added, followed by heating to 85° C. and continued reaction for 10 hours. After the thin layer chromatography plate monitored that the reaction was completed, the reaction solution was cooled to room temperature. The reaction solution was concentrated to dryness, and a crude product was purified by column chromatography (mobile phase: petroleum ether/ethyl acetate=20/1 (V/V)) to obtain 320 mg of the title compound.

### e) Preparation of 4,4,5,5-tetramethyl-2-(2H-spiro[benzofuran-3,1′-cyclopropane]-5-yl)-1,3,2-dioxaborolane

The 5-bromo-2H-spiro[benzofuran-3,1′-cyclopropane] (280 mg), bis(pinacolato)diboron (434 mg), [1,1′-bis(diphenylphosphino)ferrocene]palladium(II)dichloride (80 mg), potassium acetate (335 mg), and 1,4-dioxane (10 mL) were added to a flask, protected by nitrogen, and reacted at 90° C. for 3 hours. After the reaction, the solid was filtered out, the filtrate was collected and concentrated to dryness, and the crude product was purified by column chromatography (mobile phase: petroleum ether/ethyl acetate=25/1 (V/V)) to obtain 300 mg of the title compound.

### f) Preparation of 9-{6,7-dihydro-2H-spiro[1-benzofuran-3,1′-cyclopropane]-5-yl}-7-{2H spiro[1-benzofuran-3,1′-cyclopropane]-5-yl}-2-[(2,2,2-trifluoroethyl)amino]-8H-pyrido[1,2-a]pyrimidin-8-one

With reference to the preparation method of Example 10, the 4-(difluoromethoxy)phenyl boronic acid in step a) was replaced with 4,4,5,5-tetramethyl-2-(2H-spiro[1-benzofuran-3,1′-cyclopropane]-5-yl)-1,3,2-dioxaborolane.

¹H NMR (400 MHz, DMSO-d₆) δ 8.46 (t, J=6.3 Hz, 1H), 8.15 (d, J=7.5 Hz, 1H), 8.05 (s, 1H), 7.37 (dd, J=8.4, 1.8 Hz, 1H), 7.12-7.01 (m, 2H), 6.83-6.73 (m, 2H), 6.67 (d, J=8.2 Hz, 1H), 6.28 (d, J=7.5 Hz, 1H), 4.49 (d, J=15.3 Hz, 4H), 4.04 (dt, J=16.1, 8.0 Hz, 2H), 1.08 (d, J=4.0 Hz, 2H), 1.03 (dt, J=6.1, 3.2 Hz, 4H), 0.95 (q, J=5.0, 4.4 Hz, 2H). LCMS m/z=532 [M+1]⁺.

### Example 14: 9′-{9-[4-(difluoromethoxy)phenyl]-8-oxo-2-[(2,2,2-trifluoroethyl)amino]pyrido[1,2-a]pyrimidin-7-yl}-2′,4′-dihydrospiro[cyclopropane-1,3′-pyrazino[1,2-b]indazol]-1′-one

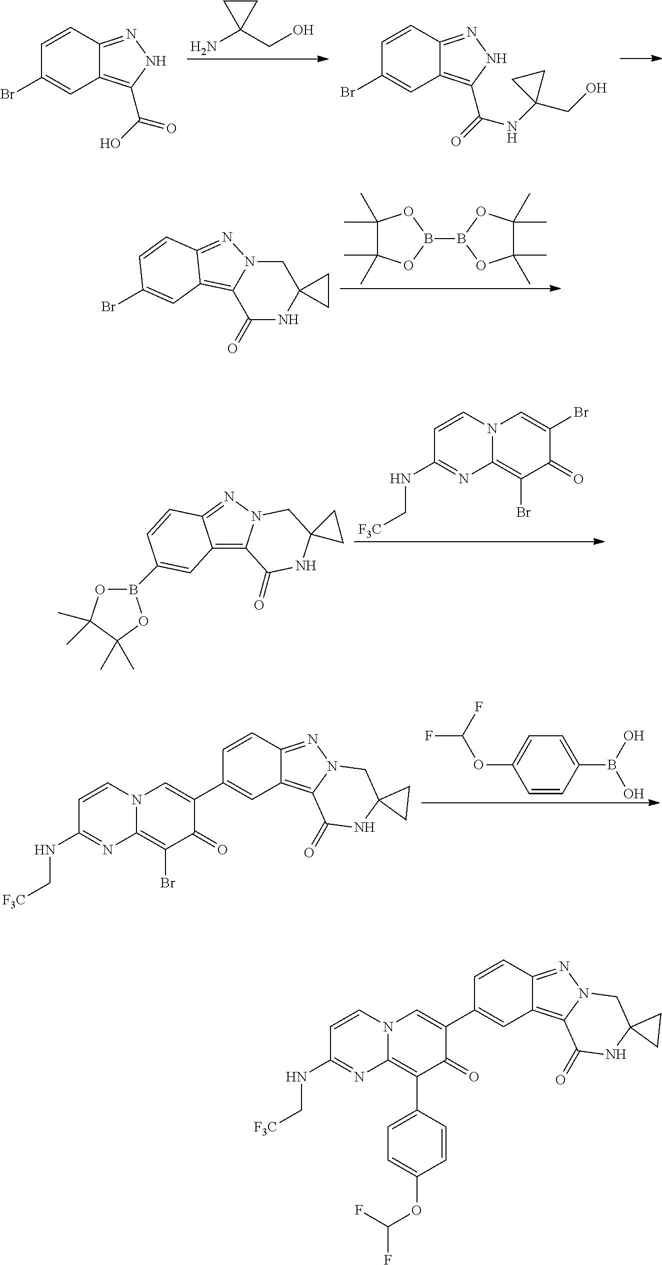

### a) Preparation of 5-bromo-N-(1-(hydroxymethyl)cyclopropyl)-2H-indazole-3-formamide

5-bromo-2H-indazole-3-carboxylic acid (6 g), (1-aminocyclopropyl) methanol hydrochloride (3.69 g), 1-hydroxybenzotriazole (4.04 g), triethylamine (5.04 g), and 1-(3-dimethylaminopropyl)-3-ethylcarbodiimide hydrochloride (5.73 g) were mixed with N,N-dimethylformamide (60 mL), and the mixture reacted at room temperature for 2 hours. The reaction was quenched with water, and the reaction solution was extracted 3 times with 100 mL of dichloromethane. Organic phases were combined and washed 3 times with 100 mL of saturated salt solution. The organic phase was concentrated to dryness, 100 mL of ether was added, filtration was performed, and the filter cake was washed twice with 100 mL of ether to obtain 2.8 g of the title compound.

MS(ESI+): 311.75 (M+H).

### b) Preparation of 9′-bromo-4′H-spiro[cyclopropane-1,3′-pyrazino[1,2-b]indazol]-1′(2′H)-one

Under nitrogen protection, the 5-bromo-N-(1-(hydroxymethyl)cyclopropyl)-2H-indazole-3-formamide (400 mg), (1-aminocyclopropyl) methanol hydrochloride (3.69 g), and triphenylphosphine (575.06 mg) were mixed with tetrahydrofuran (40 mL), di-tert-butyl azodicarboxylate (504.84 mg) was added dropwise at 0° C., and the mixture reacted at room temperature for 2 hours. The reaction was quenched with water, and the reaction solution was extracted 3 times with 100 mL of ethyl acetate for each. Organic phases were combined and washed 3 times with 100 mL of saturated salt solution for each. The organic phase was concentrated to dryness, and a crude product was purified by column chromatography (mobile phase: methanol/dichloromethane=0-1/10 (V/V)) to obtain 300 mg of the title compound.

### c) Preparation of 9′-(4,4,5,5-tetramethyl-1,3,2-dioxaborolan-2-yl)-4′H-spiro[cyclopropane-1,3′-pyrazino[1,2-b]indazol]-1′(2′H)-one

Under nitrogen protection, the 9′-bromo-4′H-spiro[cyclopropane-1,3′-pyrazino[1,2-b]indazol]-1′(2′H)-one (100 mg), bis(pinacolato)diboron (104.31 mg), and potassium acetate (100.78 mg) were mixed with 1,4-dioxane (10 mL), [1,1′-bis(diphenylphosphino)ferrocene]palladium(II)dichloride dichloromethane complex (13.94 mg) was added, and the mixture was heated to 80° C. and stirred for 2 hours. Filtration was performed after the reaction, and the filter cake was washed 3 times with 50 mL of dichloromethane for each. Organic phases were combined and concentrated to dryness, and a crude product was purified by column chromatography (mobile phase: methanol/dichloromethane=0-1/10 (V/V)) to obtain 100 mg of the title compound.

MS(ESI+): 339.90 (M+H).

### d) Preparation of 9′-{9-bromo-8-oxo-2-[(2,2,2-trifluoroethyl)amino]pyrido[1,2-a]pyrimidin-7-yl}-2′,4′-dihydrospiro[cyclopropane-1,3′-pyrazino[1,2-b]indazol]-1′-one

The 9′-(4,4,5,5-tetramethyl-1,3,2-dioxaborolan-2-yl)-2′,4′-dihydrospiro[cyclopropane-1,3′-pyrazino[1,2-b]indazol]-1′-one (100 mg), 7,9-dibromo-2-[(2,2,2-trifluoroethyl)amino]-8H-pyrido[1,2-a]pyrimidin-8-one (10 mg), [1,1′-bis(diphenylphosphino)ferrocene]palladium(II)dichloride (2.03 mg), and potassium phosphate (13.23 mg) were sequentially added to a reaction flask, then 1,4-dioxane (1 mL) and water (0.2 mL) were added, the reaction solution was stirred at 80° C. for 2 hours under nitrogen displacement protection, cooled to room temperature, and concentrated under reduced pressure to prepare sand, and the concentrate was purified by column chromatography (mobile phase: dichloromethane/methanol=10/1 (V/V)) to obtain 60 mg of the title compound.

### e) Preparation of 9′-{9-[4-(difluoromethoxy)phenyl]-8-oxo-2-[(2,2,2-trifluoroethyl)amino]pyrido[1,2-a]pyrimidin-7-yl}-2′,4′-dihydrospiro[cyclopropane-1,3′-pyrazino[1,2-b]indazol]-1′-one

The 9′-{9-bromo-8-oxo-2-[(2,2,2-trifluoroethyl)amino]pyrido[1,2-a]pyrimidin-7-yl}-2′,4′-dihydrospiro[cyclopropane-1,3′-pyrazino[1,2-b]indazol]-1′-one (60 mg), 4-(difluoromethoxy)phenyl boronic acid (42.29 mg), [1,1′-bis(diphenylphosphino)ferrocene]palladium(II)dichloride (9.16 mg), and potassium phosphate (59.70 mg) were sequentially added to a reaction flask, then 1,4-dioxane (6 mL) and water (1.2 mL) were added, the reaction solution was stirred at 80° C. for 2 hours under nitrogen displacement protection, cooled to room temperature, and concentrated under reduced pressure to dryness, and 10.1 mg of the title compound was prepared and separated. Preparation and separation conditions: (chromatographic column: YMC-Actus Triart C18, 30*150 mm, 5 μm; mobile phase A: water (10 mmol/L ammonium formate), mobile phase B: acetonitrile; flow rate: 60 mL/min; gradient: 20% B to 60% B in 8 min, 60% B; test wavelength: 220 nm; retention time (min): 7.23; column temperature: 25° C.).

¹H NMR (400 MHz, DMSO-d₆) δ 8.58 (s, 1H), 8.54 (s, 1H), 8.36 (t, J=1.3 Hz, 1H), 8.31-8.24 (m, 2H), 7.78 (d, J=8.9 Hz, 1H), 7.69 (dd, J=9.0, 1.6 Hz, 1H), 7.57-7.48 (m, 2H), 7.44-7.02 (m, 3H), 6.35 (d, J=7.5 Hz, 1H), 4.64 (s, 2H), 4.05 (t, J=8.3 Hz, 2H), 0.99 (d, J=4.1 Hz, 2H), 0.95 (d, J=4.3 Hz, 2H). LCMS m/z=597 [M+1]⁺.

### Example 15: 9′-{9-[4-(difluoromethoxy)phenyl]-8-oxo-2-[(2,2,2-trifluoroethyl)amino]pyrido[1,2-a]pyrimidin-7-yl}-2′-methyl-4′H-spiro[cyclopropane-1,3′-pyrazino[1,2-b]indazol]-1′-one

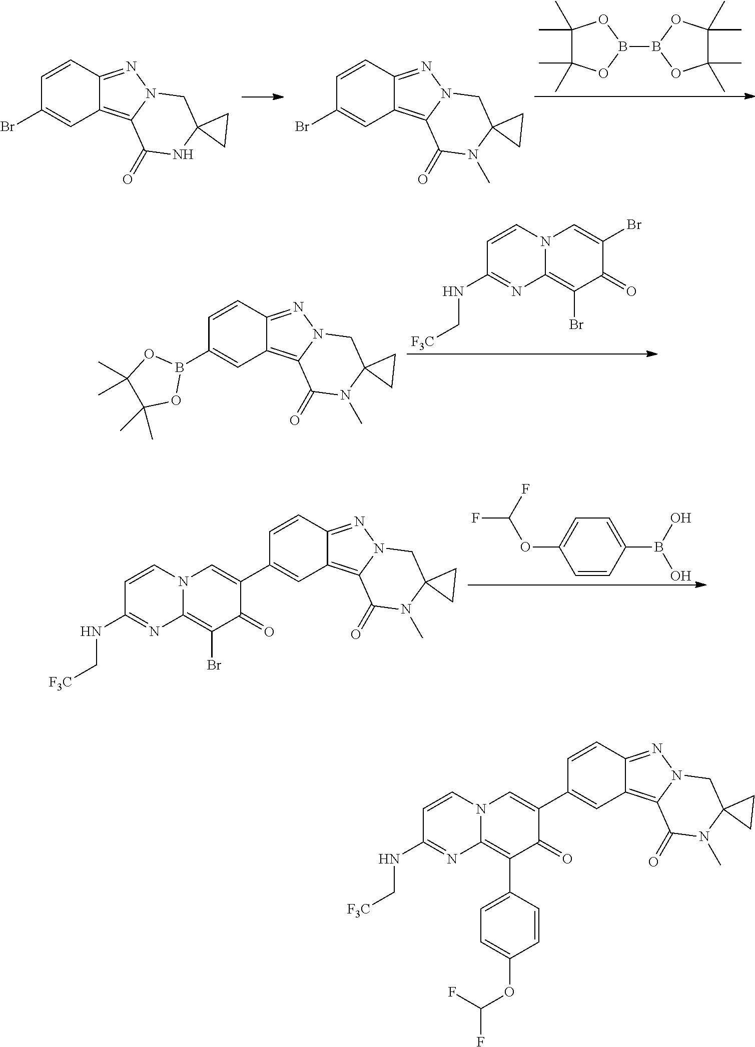

### a) Preparation of 9′-bromo-2′-methyl-4′H-spiro[cyclopropane-1,3′-pyrazino[1,2-b]indazol]-1′-one

9′-bromo-2′,4′-dihydrospiro[cyclopropane-1,3′-pyrazino[1,2-b]indazol]-1′-one (500 mg) and N,N-dimethylformamide (5 mL) were added to a reaction flask and cooled to 0° C., sodium hydride (82.15 mg) was added, iodomethane (364.40 mg) was added dropwise at 25° C., the mixture was stirred for 1 hour, the reaction was quenched with water at 0° C., the reaction solution was concentrated under reduced pressure and extracted with ethyl acetate (3×20 mL), the combined organic layer was washed 3 times with saturated sodium bicarbonate and washed with saturated salt solution (1×20 mL), drying with anhydrous sodium sulfate and filtration were performed, and the filtrate was concentrated under reduced pressure to obtain 460 mg of the title compound.

### b) Preparation of 2′-methyl-9′-4,4,5,5-tetramethyl-1,3,2-dioxaborolan-2-yl)-4′H-spiro[cyclopropane-1,3′-pyrazino[1,2-b]indazol]-1′-one

The 9′-bromo-2′-methyl-4′H-spiro[cyclopropane-1,3′-pyrazino[1,2-b]indazol]-1′-one (200 mg), 1,4-dioxane (20 mL), bis(pinacolato)diboron (199 mg), potassium acetate (192.33 mg), and [1,1′-bis(diphenylphosphino)ferrocene]palladium(II)dichloride dichloromethane complex (26.61 mg) were added to a reaction flask and stirred at 80° C. for 2 hours under nitrogen protection, the obtained mixture was concentrated under reduced pressure, and the concentrate was purified by column chromatography (mobile phase: petroleum ether/ethyl acetate=2/1 (V/V)) to obtain 180 mg of the title compound.

### c) Preparation of 9′-{9-[4-(difluoromethoxy)phenyl]-8-oxo-2-[(2,2,2-trifluoroethyl)amino]pyrido[1,2-a]pyrimidin-7-yl}-2′-methyl-4′H-spiro[cyclopropane-1,3′-pyrazino[1,2-b]indazol]-1′-one

With reference to the preparation method of Example 14, the 9′-(4,4,5,5-tetramethyl-1,3,2-dioxaborolan-2-yl)-2′,4′-dihydrospiro[cyclopropane-1,3′-pyrazino[1,2-b]indazol]-1′-one in step a) was replaced with the 2′-methyl-9′-4,4,5,5-tetramethyl-1,3,2-dioxaborolan-2-yl)-4′H-spiro[cyclopropane-1,3′-pyrazino[1,2-b]indazol]-1′-one.

¹H NMR (400 MHz, DMSO-d₆) δ 8.59 (t, J=6.1 Hz, 1H), 8.35 (t, J=1.3 Hz, 1H), 8.28 (d, J=7.2 Hz, 2H), 7.77 (dd, J=9.0, 0.9 Hz, 1H), 7.68 (dd, J=9.0, 1.6 Hz, 1H), 7.55-7.50 (m, 2H), 7.24 (t, J=76.4 Hz, 1H), 7.13-7.07 (m, 2H), 6.36 (d, J=7.5 Hz, 1H), 4.67 (s, 2H), 4.14-3.98 (m, 2H), 2.89 (s, 3H), 1.27 (q, J=6.9 Hz, 2H), 1.05-0.97 (m, 2H). LCMS m/z=611 [M+1]⁺.

### Example 16: 9′-{9-[4-(difluoromethoxy)phenyl]-8-oxo-2-[(2,2,2-trifluoroethyl)amino]pyrido[1,2-a]pyrimidin-7-yl}-2′,4′-dihydrospiro[cyclopropane-1,1′-pyrazino[1,2-b]indazol]-3′-one

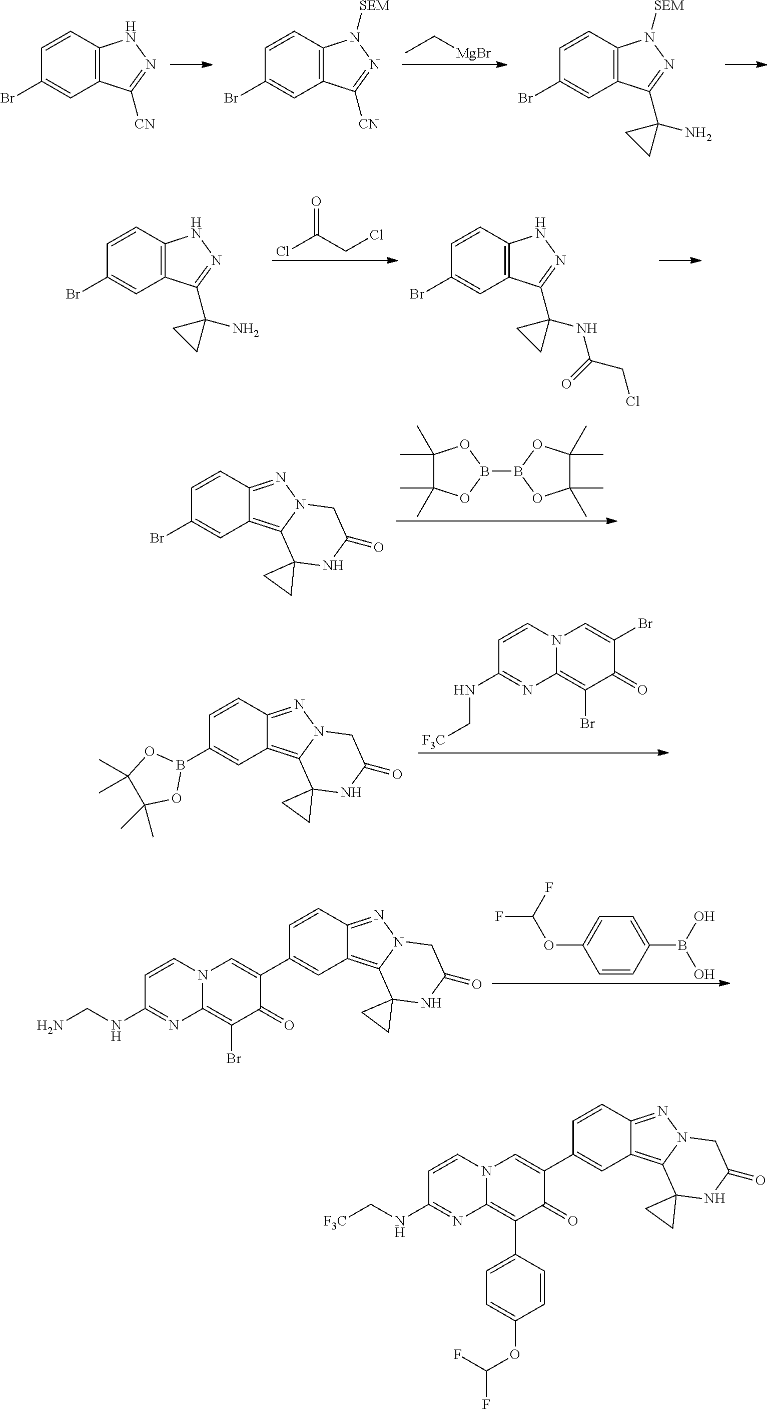

### a) Preparation of 5-bromo-1-(2-(trimethylsilyl) ethoxy)methyl)-1H-indazole-3-carbonitrile

Under nitrogen protection, sodium hydride (2.81 g) was mixed with tetrahydrofuran (110 mL), a tetrahydrofuran (85 mL) solution of 5-bromo-1H-indazole-3-carbonitrile (13 g) was added in batches at −20° C., the solution was stirred at −20° C. for 45 minutes, and then [2-(chloromethoxy)ethyl]trimethylsilane (19.52 g) was added. The solution was continuously stirred at −20° C. for 45 minutes and then stirred at room temperature for 30 minutes. The reaction was quenched with water after the reaction was completed. The aqueous phase was extracted 3 times with 70 mL of ethyl acetate for each. Organic phases were combined and concentrated to dryness, and a crude product was purified by column chromatography (mobile phase: petroleum ether/ethyl acetate=5/1 (V/V)) to obtain 20.73 g of the title compound.

### b) Preparation of 1-(5-bromo-1-((2-(trimethylsilyl)ethoxy)methyl)-1H-indazol-3-yl)cyclopropyl-1-amine

Under nitrogen protection, tetraisopropyl titanate (1.56 g) was mixed with ether (50 mL), an ethyl magnesium bromide (1.46 g) solution was added dropwise in batches at −70° C., the solution was stirred at −70° C. for 1 hour, then the 5-bromo-1-(2-(trimethylsilyl) ethoxy)methyl)-1H-indazole-3-carbonitrile (1.76 g) was added, and the solution was stirred at room temperature for 1 hour. After the reaction was completed, 1N hydrochloric acid (15 mL) and 10% sodium hydroxide solution (50 mL) were added for quenching. The aqueous phase was extracted 3 times with 70 mL of ethyl acetate for each. Organic phases were combined and concentrated to dryness, and a crude product was purified by column chromatography (mobile phase: petroleum ether/ethyl acetate=3/1 (V/V)) to obtain 1.22 g of the title compound. MS(ESI+): 382 (M+H).

### c) Preparation of 1-(5-bromo-1H-indazol-3-yl)cyclopropyl-1-amine

Under nitrogen protection, the 1-(5-bromo-1-((2-(trimethylsilyl) ethoxy)methyl)-1H-indazol-3-yl)cyclopropyl-1-amine (10 g) was mixed with N,N-dimethylformamide (200 mL), tetrabutylammonium bromide (1M tetrahydrofuran solution, 130 mL) and ethylenediamine (15.72 g) were added, and the mixture was heated to 70° C., stirred for 5 hours, and then cooled to room temperature. 500 mL of ethyl acetate was added to the reaction solution for diluting, followed by washing twice with 200 mL of water. The aqueous phase was extracted 3 times with 100 mL of ethyl acetate for each. Organic phases were combined and washed 3 times with 100 mL of saturated salt solution for each. The organic phase was concentrated to dryness, and a crude product was purified by column chromatography (mobile phase: methanol/dichloromethane=0-1/10 (V/V)) to obtain 5 g of the title compound. MS(ESI+): 252 (M+H).

### d) Preparation of N-(1-(5-bromo-1H-indazol-3-yl)cyclopropyl)-2-chloroacetamide

Under nitrogen protection, the 1-(5-bromo-1H-indazol-3-yl)cyclopropyl-1-amine (4 g) and triethylamine (3.21 g) were mixed with dichloromethane (120 mL), chloroacetyl chloride (2.33 g) was added dropwise at −10° C., and the mixture was stirred at −10° C. for 4 hours. After the reaction was completed, water was added at 0° C. for quenching. The reaction solution was extracted 3 times with 100 mL of mixed solution of methanol/dichloromethane=1/10 (V/V). Organic phases were combined and washed with 100 mL of saturated salt solution. The organic phase was concentrated to dryness, and a crude product was purified by column chromatography (mobile phase: methanol/dichloromethane=0-1/10 (V/V)) to obtain 2.5 g of the title compound. MS(ESI+): 328 (M+H).

### e) Preparation of 9′-bromo-2′H-spiro[cyclopropane-1,1′-pyrazino[1,2-b]indazol]-3′(4′H)-one

Under nitrogen protection, the N-(1-(5-bromo-1H-indazol-3-yl)cyclopropyl)-2-chloroacetamide (2 g) was mixed with N-methylpyrrolidone (200 mL), potassium carbonate (1.68 g) was added, and the mixture was heated to 80° C., stirred for 5 hours, and then cooled to room temperature. 300 mL of ethyl acetate and 300 mL of water were added to the reaction solution for diluting, and the mixed solution was extracted 3 times with 300 mL of ethyl acetate. Organic phases were combined and washed twice with 200 mL of saturated salt solution. The organic phase was concentrated to 20 mL and filtered, and the filter cake was washed 3 times with 5 mL of ethyl acetate to obtain the title compound. MS(ESI+): 292 (M+H).

### f) Preparation of 9′-(4,4,5,5-tetramethyl-1,3,2-dioxaborolan-2-yl)-2′H-spiro[cyclopropane-1,1′-pyrazino[1,2-b]indazol]-3′(4′H)-one

Under nitrogen protection, the 9′-bromo-2′H-spiro[cyclopropane-1,1′-pyrazino[1,2-b]indazol]-3′(4′H)-one (160 mg), bis(pinacolato)diboron (167 mg), and potassium acetate (134.48 mg) were mixed with 1,4-dioxane (3 mL), [1,1′-bis(diphenylphosphino)ferrocene]palladium(II)dichloride dichloromethane complex (44.62 mg) was added, and the mixture was heated to 80° C. and stirred for 2 hours. Filtration was performed after the reaction, and the filter cake was washed 3 times with 30 mL of dichloromethane. Organic phases were combined and concentrated to dryness, and a crude product was purified by column chromatography (mobile phase: methanol/dichloromethane=0-1/10 (V/V)) to obtain 130 mg of the title compound. MS(ESI+): 340.05 (M+H).

### g) Preparation of 9′-{9-[4-(difluoromethoxy)phenyl]-8-oxo-2-[(2,2,2-trifluoroethyl)amino]pyrido[1,2-a]pyrimidin-7-yl}-2′,4′-dihydrospiro[cyclopropane-1,1′-pyrazino[1,2-b]indazol]-3′-one

With reference to the preparation method of Example 14, the 9′-(4,4,5,5-tetramethyl-1,3,2-dioxaborolan-2-yl)-2′,4′-dihydrospiro[cyclopropane-1,3′-pyrazino[1,2-b]indazol]-1′-one in step d) was replaced with 9′-(4,4,5,5-tetramethyl-1,3,2-dioxaborolan-2-yl)-2′,4′-dihydrospiro[cyclopropane-1,1′-pyrazino[1,2-b]indazol]-3′-one.

¹H NMR (400 MHz, DMSO-d₆) δ 8.89 (s, 1H), 8.57 (t, J=6.3 Hz, 1H), 8.23 (d, J=12.6 Hz, 2H), 7.84 (d, J=1.5 Hz, 1H), 7.58 (d, J=1.2 Hz, 2H), 7.52-7.44 (m, 2H), 7.23 (t, J=74.0 Hz, 1H), 7.15-7.04 (m, 2H), 6.35 (d, J=7.5 Hz, 1H), 5.16 (s, 2H), 4.03 (dt, J=16.0, 8.1 Hz, 2H), 1.78-1.72 (m, 2H), 1.56-1.45 (m, 2H). LCMS m/z=597 [M-+1]⁺.

### Example 17: 9-[4-(difluoromethoxy)phenyl]-7-{2H-spiro[1-benzofuran-3,1′-cyclopropane]-5-yl}-2-[(2,2,2-trifluoroethyl)amino]-8H-pyrido[1,2-a]pyrimidin-8-one

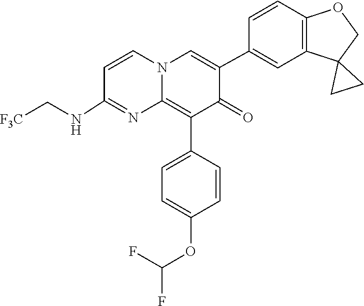 [SMILES: O=c1c(-c2ccc3c(c2)C2(CC2)CO3)cn2ccc(NCC(F)(F)F)nc2c1-c1ccc(OC(F)F)cc1]

With reference to the preparation method of Example 14, the 9′-(4,4,5,5-tetramethyl-1,3,2-dioxaborolan-2-yl)-2′,4′-dihydrospiro[cyclopropane-1,3′-pyrazino[1,2-b]indazol]-1′-one in step a) was replaced with 4,4,5,5-tetramethyl-2-{2H-spiro[1-benzofuran-3,1′-cyclopropane-5-yl}-1,3,2-dioxaborolane.

¹H NMR (400 MHz, DMSO-d₆) δ 8.53 (t, J=6.2 Hz, 1H), 8.19 (d, J=7.5 Hz, 1H), 8.10 (s, 1H), 7.50-7.42 (m, 2H), 7.41-7.34 (m, 1H), 7.22 (t, J=74.4 Hz, 1H), 7.14 (d, J=1.9 Hz, 1H), 7.11-7.05 (m, 2H), 6.80 (d, J=8.3 Hz, 1H), 6.32 (d, J=7.4 Hz, 1H), 4.51 (s, 2H), 4.10-3.89 (m, 2H), 1.12-1.06 (m, 2H), 1.06-0.97 (m, 2H). LCMS m/z=530.05 [M+1]⁺.

### Example 18: 7-(2-amino-1,3-benzothiazol-6-yl)-9-bromo-2-[(2,2,2-trifluoroethyl)amino]-8H-pyrido[1,2-a]pyrimidin-8-one

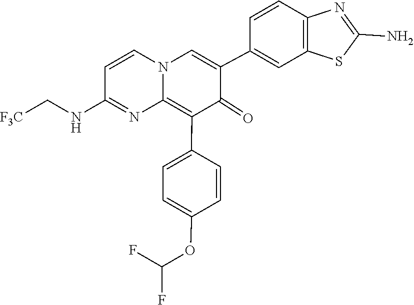 [SMILES: Nc1nc2ccc(-c3cn4ccc(NCC(F)(F)F)nc4c(-c4ccc(OC(F)F)cc4)c3=O)cc2s1]

With reference to the preparation method of Example 14, the 9′-(4,4,5,5-tetramethyl-1,3,2-dioxaborolan-2-yl)-2′,4′-dihydrospiro[cyclopropane-1,3′-pyrazino[1,2-b]indazol]-1′-one in step a) was replaced with 6-(4,4,5,5-tetramethyl-1,3,2-dioxaborolan-2-yl)-1,3-benzothiazol-2-amine.

¹H NMR (400 MHz, DMSO-d₆) δ 8.55 (t, J=6.3 Hz, 1H), 8.22 (d, J=8.2 Hz, 2H), 8.03 (d, J=1.7 Hz, 1H), 7.57-7.46 (m, 5H), 7.36 (d, J=8.4 Hz, 1H), 7.23 (t, J=72 Hz, 1H), 7.13-7.02 (m, 2H), 6.34 (d, J=7.5 Hz, 1H), 4.08-3.98 (m, 2H). LCMS m/z=534 [M+1]⁺.

### Example 19: 2-(5-{9-[4-(difluoromethoxy)phenyl]-8-oxo-2-[(2,2,2-trifluoroethyl)amino]pyrido[1,2-a]pyrimidin-7-yl}indazol-2-yl)acetonitrile

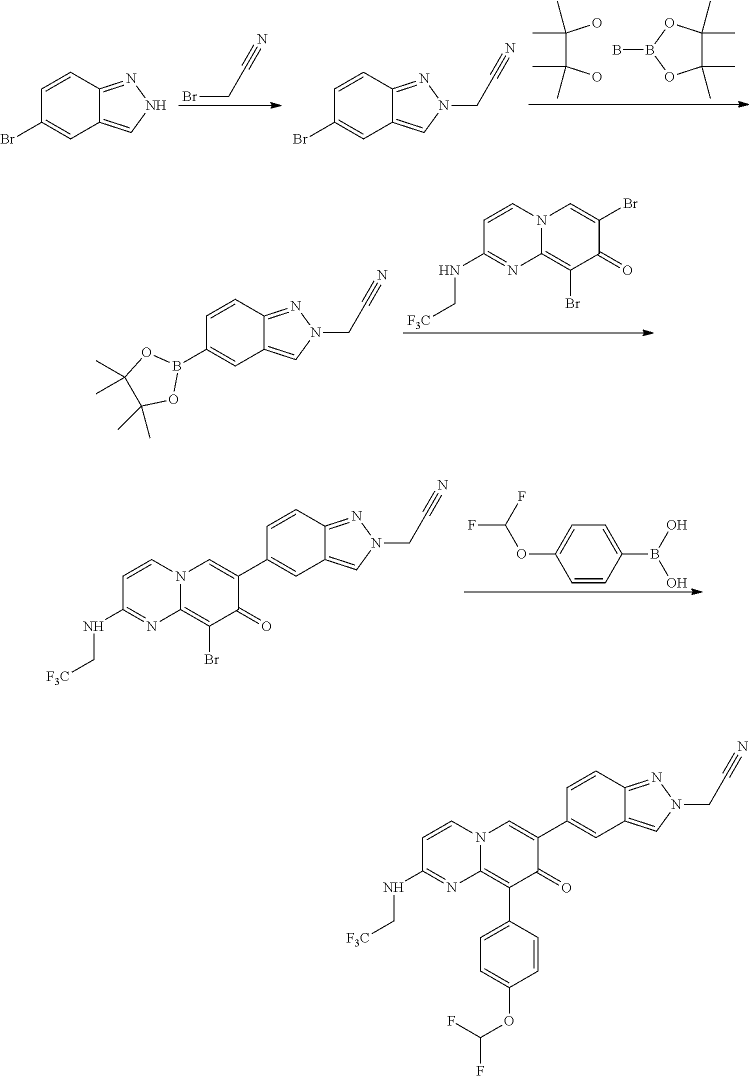

### a) Preparation of 2-(5-bromoindazol-2-yl) acetonitrile

5-bromo-2H-indazole (6 g), 2-bromoacetonitrile (5.48 g), and N-methylpyrrolidone (60 mL) were added to a reaction flask and stirred at 120° C. for 1 hour under nitrogen protection; the reaction solution was cooled to room temperature, diluted with water (180 mL), and extracted with ethyl acetate (3×200 mL); the organic layer was washed with saturated salt solution (3×200 mL), dried with anhydrous sodium sulfate, and filtered; the filtrate was concentrated under reduced pressure; and the concentrate was purified by column chromatography (mobile phase: petroleum ether/ethyl acetate=5/1 (V/V)) to obtain 6.29 g of the title compound.

### b) Preparation of 2-[5-(4,4,5,5-tetramethyl-1,3,2-dioxaborolan-2-yl)indazol-2-yl]acetonitrile

The 2-(5-bromoindazol-2-yl) acetonitrile (200 mg), bis(pinacolato)diboron (322.71 mg), [1,1′-bis(diphenylphosphino)ferrocene]palladium(II)dichloride (69.19 mg), 1,4-dioxane (8 mL), and potassium acetate (249.44 mg) were added to a reaction flask and stirred at 80° C. for 4 hours under nitrogen protection, water (15 mL) was added to quench the reaction, the reaction solution was extracted with ethyl acetate (3×25 mL), the organic layer was washed with saturated salt solution (1×20 mL), dried with anhydrous sodium sulfate, and filtered, the filtrate was concentrated under is reduced pressure, and the concentrate was purified by column chromatography (mobile phase: petroleum ether/ethyl acetate=3/1 (V/V)) to obtain 210 mg of the title compound.

### c) Preparation of 2-(5-{9-[4-(difluoromethoxy)phenyl]-8-oxo-2-[(2,2,2-trifluoroethyl)amino]pyrido[1,2-a]pyrimidin-7-yl}indazol-2-yl) acetonitrile

With reference to the preparation method of Example 14, the 9′-(4,4,5,5-tetramethyl-1,3,2-dioxaborolan-2-yl)-2′,4′-dihydrospiro[cyclopropane-1,3′-pyrazino[1,2-b]indazol]-1′-one in step a) was replaced with the 2-[5-(4,4,5,5-tetramethyl-1,3,2-dioxaborolan-2-yl) indazol-2-yl]acetonitrile.

¹H NMR (400 MHz, DMSO-d₆) δ 8.61-8.53 (m, 2H), 8.24 (d, J=8.2 Hz, 2H), 8.12 (t, J=1.3 Hz, 1H), 7.67 (dt, J=9.1, 1.0 Hz, 1H), 7.61 (dd, J=9.0, 1.6 Hz, 1H), 7.54-7.48 (m, 2H), 7.24 (m, 1H), 7.09 (d, J=8.6 Hz, 2H), 6.36 (d, J=7.5 Hz, 1H), 5.86 (s, 2H), 4.05 (dd, J=9.7, 6.5 Hz, 2H). LCMS m/z=541 [M+1].

### Example 20: 9-(2,2-difluoro-1,3-benzodioxol-5-yl)-7-(2-methyl-2H-indazol-5-yl)-2-[(2,2,2-trifluoroethyl)amino]-8H-pyrido[1,2-a]pyrimidin-8-one

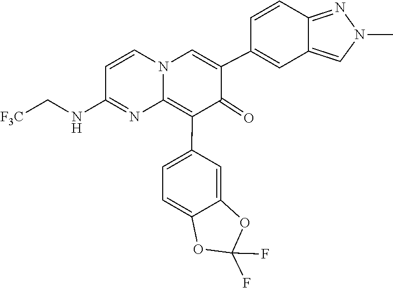 [SMILES: Cn1cc2cc(-c3cn4ccc(NCC(F)(F)F)nc4c(-c4ccc5c(c4)OC(F)(F)O5)c3=O)ccc2n1]

With reference to the preparation method of Example 14, the 9′-(4,4,5,5-tetramethyl-1,3,2-dioxaborolan-2-yl)-2′,4′-dihydrospiro[cyclopropane-1,3′-pyrazino[1,2-b]indazol]-1′-one in step a) was replaced with 2-methyl-2H-indazol-5-yl boronic acid, and the 4-(difluoromethoxy)phenyl boronic acid in step b) was replaced with 2,2-difluoro-1,3-benzodioxol-5-yl boronic acid.

¹H NMR (400 MHz, DMSO-d₆) δ 8.62 (t, J=6.3 Hz, 1H), 8.38 (s, 1H), 8.25 (d, J=7.1 Hz, 2H), 8.06 (d, J=1.5 Hz, 1H), 7.59 (d, J=9.0 Hz, 1H), 7.53 (dd, J=9.0, 1.6 Hz, 1H), 7.39 (d, J=1.5 Hz, 1H), 7.35-7.24 (m, 2H), 6.36 (d, J=7.5 Hz, 1H), 4.18 (s, 3H), 4.11-3.97 (m, 2H). LCMS m/z=530 [M+1]⁺.

### Example 21: 7,9-bis[4-(difluoromethoxy)phenyl]-1H-pyrido[1,2-a]pyrimidin-2,8-dione

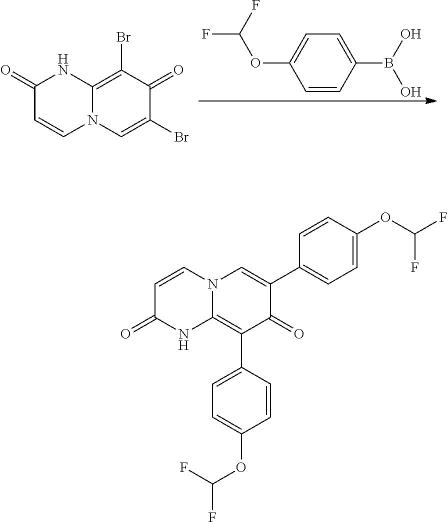

With reference to the preparation method of Example 10, the 7,9-dibromo-2-[(2,2,2-trifluoroethyl)amino]-8H-pyrido[1,2-a]pyrimidin-8-one in step a) was replaced with 7,9-dibromo-1H-pyrido[1,2-a]pyrimidin-2,8-dione.

¹H NMR (400 MHz, DMSO-d₆) δ 10.34 (s, 1H), 8.22 (s, 1H), 8.12 (d, J=7.9 Hz, 1H), 7.74-7.65 (m, 2H), 7.31 (d, J=8.6 Hz, 2H), 7.31 (t, J=74.0 Hz, 1H), 7.22 (dd, J=8.7, 3.0 Hz, 4H), 6.04 (d, J=7.9 Hz, 1H). LCMS m/z=447[M+1]⁺.

### Example 22: 9-[4-(difluoromethoxy)phenyl]-7-(2-methyl-2H-indazol-5-yl)-2-(2,2,2-trifluoroethoxy)-8H-pyrido[1,2-a]pyrimidin-8-one

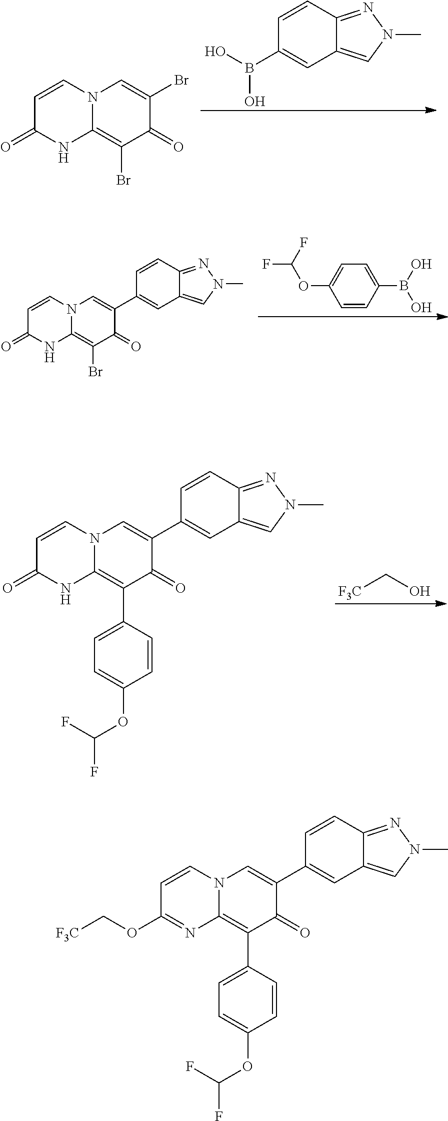

### a) Preparation of 9-bromo-7-(2-methyl-2H-indazol-5-yl)-1H-pyrido[1,2-a]pyrimidin-2,8-dione

7,9-dibromo-1H-pyrido[1,2-a]pyrimidin-2,8-dione (500 mg), 2-methyl-2H-indazol-5-yl boronic acid (412.5 mg), N,N-dimethylformamide (50 mL), water (10 mL), potassium phosphate (995.2 mg), [1,1′-bis(diphenylphosphino)ferrocene]palladium(II)dichloride dichloromethane complex (127.3 mg) were added to a reaction flask, stirred at 80° C. for 2 hours under nitrogen protection, concentrated and evaporated under reduced pressure to dryness, followed by column chromatography purification (mobile phase: dichloromethane/methanol=10/1 (V/V)) to obtain 280 mg of the title compound.

### b) Preparation of 9-[4-(difluoromethoxy)phenyl]-7-(2-methyl-2H-indazol-5-yl)-1H-pyrido[1,2-a]pyrimidin-2,8-dione

The 9-bromo-7-(2-methyl-2H-indazol-5-yl)-1H-pyrido[1,2-a]pyrimidin-2,8-dione (280 mg), 1,4-dioxane (28 mL), water (5.60 mL), (difluoromethoxy)phenyl boronic acid (283.5 mg), potassium phosphate (400.3 mg), and [1,1′-bis(diphenylphosphino)ferrocene]palladium(II)dichloride dichloromethane complex (61.5 mg) were added to a reaction flask, stirred at 80° C. for 2 hours under nitrogen protection, concentrated and evaporated under reduced pressure to dryness, followed by column chromatography purification (mobile phase: dichloromethane/methanol=10/1 (V/V)) to obtain 150 mg of the title compound.

### c) Preparation of 9-[4-(difluoromethoxy)phenyl]-7-(2-methyl-2H-indazol-5-yl)-2-(2,2,2-trifluoroethoxy)-8H-pyrido[1,2-a]pyrimidin-8-one

The 9-[4-(difluoromethoxy)phenyl]-7-(2-methyl-2H-indazol-5-yl)-1H-pyrido[1,2-a]pyrimidin-2,8-dione (20 mg), 1H-benzotriazol-1-yloxytris(dimethylamino) hexafluorophosphate (40.73 mg), and cesium carbonate (30 mg) were sequentially added to a reaction flask, stirred at room temperature for 10 minutes, then added with cesium carbonate (30 mg) and trifluoroethanol (92.12 mg), and stirred at room temperature for 2 hours. The LCMS test reaction was completed, and the reaction solution was concentrated under reduced pressure to dryness to prepare and separate 10 mg of the title compound. Preparation and separation conditions: (chromatographic column: XBridge Prep OBD C18, 30*150 mm, 5 μm; mobile phase A: water (10 mmol/L ammonium formate), mobile phase B: acetonitrile; flow rate: 60 mL/min; gradient: 25% B to 65% B (0-8), 65% B; test wavelength: 220 nm; retention time (min): 7.99; column temperature: 25° C.).

¹H NMR (400 MHz, DMSO-d₆) δ 8.57 (d, J=7.8 Hz, 1H), 8.47 (s, 1H), 8.42 (s, 1H), 8.11 (s, 1H), 7.62 (d, J=9.0 Hz, 1H), 7.55 (dd, J=8.8, 6.2 Hz, 3H), 7.28 (t, J=74.0 Hz, 1H), 7.16 (d, J=8.4 Hz, 2H), 6.69 (d, J=7.4 Hz, 1H), 4.84 (q, J=8.9 Hz, 2H), 4.19 (s, 3H). LCMS m/z=517 [M+1]⁺.

### Example 23: 2-cyclopropyl-7,9-bis[4-(difluoromethoxy)phenyl]-8H-pyrido[1,2-a]pyrimidin-8-one

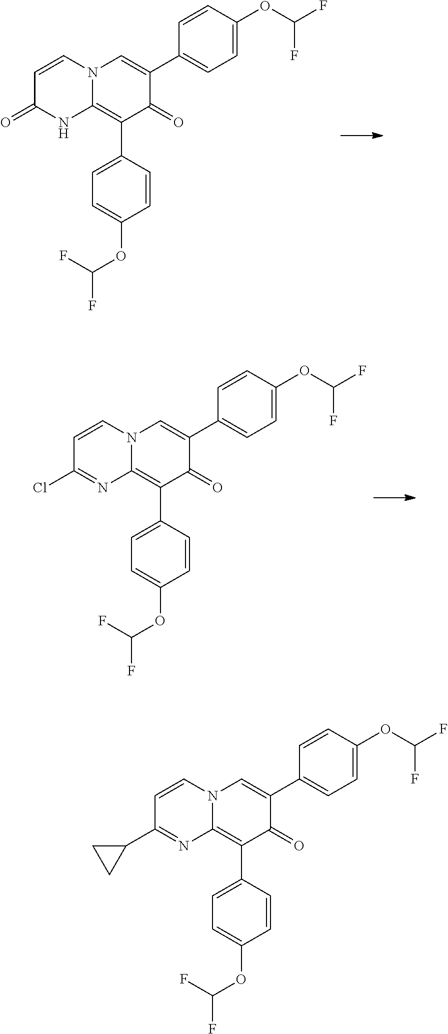 [SMILES: O=c1ccn2cc(-c3ccc(OC(F)F)cc3)c(=O)c(-c3ccc(OC(F)F)cc3)c2[nH]1 ; O=c1c(-c2ccc(OC(F)F)cc2)cn2ccc(Cl)nc2c1-c1ccc(OC(F)F)cc1 ; O=c1c(-c2ccc(OC(F)F)cc2)cn2ccc(C3CC3)nc2c1-c1ccc(OC(F)F)cc1]

### a) Preparation of 2-chloro-7,9-bis[4-(difluoromethoxy)phenyl]-8H-pyrido[1,2-a]pyrimidin-8-one

7,9-bis[4-(difluoromethoxy)phenyl]-1H pyrido[1,2-a]pyrimidin-2,8-dione (50 mg) and sulfoxide chloride (5 mL) were sequentially added to a reaction flask and stirred at 50° C. for 2 days, the reaction solution was filtered, the filtrate was concentrated under reduced pressure, the concentrate was diluted with dichloromethane (20 mL), the organic phase was washed with saturated salt solution and saturated sodium bicarbonate and dried with anhydrous sodium sulfate, and the filtrate was concentrated under reduced pressure to prepare sand, followed by column chromatography purification (mobile phase: petroleum ether/ethyl acetate=2/1 (V/V)) to obtain 28 mg of the title compound.

### b) Preparation of 2-cyclopropyl-7,9-bis[4-(difluoromethoxy)phenyl]-8H-pyrido[1,2-a]pyrimidin-8-one

The 2-chloro-7,9-bis[4-(difluoromethoxy)phenyl]-8H-pyrido[1,2-a]pyrimidin-8-one (19 mg), cyclopropyl boronic acid (7.02 mg), water (0.2 mL), 1,4-dioxane (1 mL), potassium carbonate (11.30 mg), and bis(triphenylphosphino) palladium(II)dichloride (5.74 mg) were sequentially added to a reaction flask and stirred at 80° C. for 2 hours under nitrogen displacement protection, and the obtained reaction solution was concentrated under reduced pressure to prepare and separate 2.4 mg of the title compound. Preparation and separation conditions: (chromatographic column: YMC-Actus Triart C18, 30*150 mm, 5 μm; mobile phase A: water (10 mmol/L ammonium formate), mobile phase B: acetonitrile; flow rate: 60 mL/min; gradient: 45% B to 69% B in 8 min, 69% B; test wavelength: 220 nm; retention time (min): 7.50; column temperature: 25° C.).

¹H NMR (400 MHz, DMSO-d₆) δ 8.44-8.37 (m, 2H), 7.84-7.76 (m, 2H), 7.50-7.42 (m, 2H), 7.26 (dd, J=9.3, 7.4 Hz, 3H), 7.17-7.05 (m, 3H), 6.82 (d, J=7.2 Hz, 1H), 2.04 (tt, J=8.3, 4.5 Hz, 1H), 1.05 (dq, J=6.9, 3.7 Hz, 2H), 0.93 (p, J=3.8 Hz, 2H). LCMS m/z=471 [M+1]⁺.

### Example 24: 7,9-bis[4-(difluoromethoxy)phenyl]-2-(2,2,2-trifluoroethoxy)-8H-pyrido[1,2-a]pyrimidin-8-one

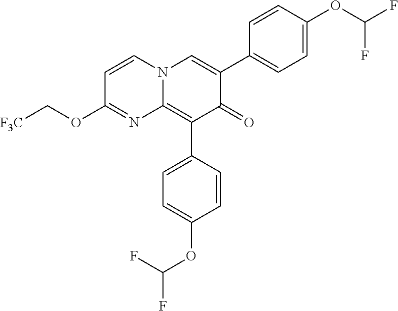 [SMILES: O=c1c(-c2ccc(OC(F)F)cc2)cn2ccc(OCC(F)(F)F)nc2c1-c1ccc(OC(F)F)cc1]

With reference to the preparation method of Example 22, the 9-[4-(difluoromethoxy)phenyl]-7-(2-methyl-2H-indazol-5-yl)-1H-pyrido[1,2-a]pyrimidin-2,8-dione in step a) was replaced with 7,9-bis[4-(difluoromethoxy)phenyl]-1H-pyrido[1,2-a]pyrimidin-2,8-dione.

¹H NMR (400 MHz, DMSO-d₆) δ 8.54 (d, J=7.6 Hz, 1H), 8.45 (s, 1H), 7.84-7.74 (m, 2H), 7.58-7.44 (m, 2H), 7.31-7.23 (m, 3H), 7.18-7.07 (m, 3H), 6.70 (d, J=7.4 Hz, 1H), 4.83 (q, J=8.9 Hz, 2H). LCMS m/z=529[M+1]⁺.

### Example 25: 2-cyclopropyl-9-[4-(difluoromethoxy)phenyl]-7-(2-methyl-2H-indazol-5-yl)-8H-pyrido[1,2-a]pyrimidin-8-one

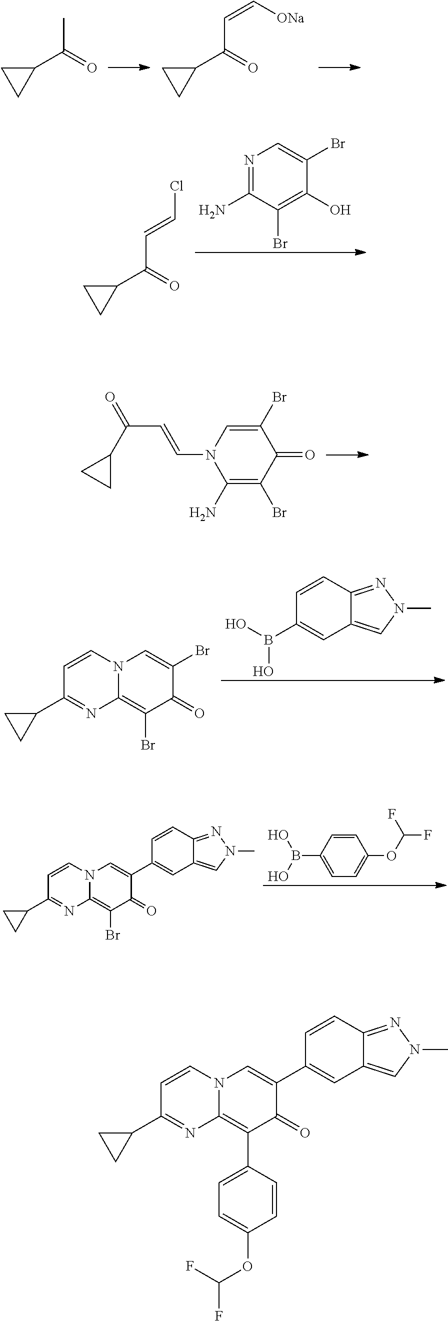

### a) Preparation of sodium 3-cyclopropyl-3-oxoprop-1-en-1-olate

Under nitrogen protection, sodium hydride (2.85 g) was added in batches to a tetrahydrofuran (150 mL) solution of cyclopropyl methyl ketone (10 g) at 0° C., the reaction system was stirred at 0° C. for 1 hour, then methyl formate (7.85 g) was added dropwise, and the reaction system was stirred overnight at room temperature. After the reaction was completed, the reaction solution was concentrated under reduced pressure and filtered to collect washed solid, and the solid was washed with petroleum ether (3×30 mL) and methyl tert-butyl ether (3×30 mL) to obtain 10 g of the title compound.

### b) Preparation of (2E)-3-chloro-1-cyclopropylprop-2-en-1-one

Under nitrogen protection, (2Z)-1-cyclopropyl-3-(dioxy) prop-2-en-1-one (15 g) was added to a solution of dichloromethane (300 mL), and oxalyl chloride (28.39 g) was added dropwise. The reaction system was stirred at room temperature for 2 hours. After the reaction was completed, saturated sodium bicarbonate (100 ml) was added to quench the reaction, the reaction solution was extracted with dichloromethane (3×100 mL), organic layers were combined, washed with saturated salt solution (1×200 mL), dried with anhydrous sodium sulfate, and filtered, and the filtrate was concentrated under reduced pressure, followed by column chromatography purification (mobile phase: dichloromethane/petroleum ether=3/10 (V/V)) and elution to obtain 6.5 g of the title compound.

### c) Preparation of 2-amino-3,5-dibromo-1-[(1E)-3-cyclopropyl-3-oxoprop-1-en-1-yl]pyridin-4-one

The (2E)-3-chloro-1-cyclopropylprop-2-en-1-one (6.5 g) and 2-amino-3,5-dibromopyridin-4-ol (14.67 g) were added to a reaction flask, N,N-dimethylformamide (130 mL) and potassium carbonate (20.64 g) were added under room temperature nitrogen protection, and the mixture was stirred overnight at room temperature. After the reaction was completed, the reaction solution was diluted with water (200 mL) and extracted with ethyl acetate (3×150 mL), organic phases were combined, washed with saturated salt solution (2×300 mL), dried with anhydrous sodium sulfate, and filtered, and the filtrate was concentrated under reduced pressure. Column chromatography purification (mobile phase: ethyl acetate/petroleum ether=1/1 (V/V)) was performed to obtain 5.3 g of the title compound.

### d) Preparation of 7,9-dibromo-2-cyclopropyl-8H-pyrido[1,2-a]pyrimidin-8-one

The 2-amino-3,5-dibromo-1-[(1E)-3-cyclopropyl-3-oxoprop-1-en-1-yl]pyridin-4-one (5.3 g) was added to a reaction flask, hydrogen chloride (4 M, 50 mL) and 1,4-dioxane (50 mL) were added under nitrogen protection, the solution was stirred at room temperature for 1 hour, the obtained mixture was concentrated under reduced pressure, the concentrate was diluted with ethyl acetate (20 mL), the pH was adjusted to 8 with saturated sodium bicarbonate aqueous solution, the solution was extracted with ethyl acetate (3×100 mL), organic phases were combined, washed with saturated salt solution (1×40 mL), dried with anhydrous sodium sulfate, and filtered, and the filtrate was concentrated under reduced pressure. Column chromatography purification (mobile phase: methanol/dichloromethane=1/10 (V/V)) was performed to obtain 2.3 g of the title compound.

### e) Preparation of 9-bromo-2-cyclopropyl-7-(2-methyl-2H-indazol-5-yl)-8H-pyrido[1,2-a]pyrimidin-8-one

The 7,9-dibromo-2-cyclopropyl-8H-pyrido[1,2-a]pyrimidin-8-one (100 mg), 2-methyl-2H-indazol-5-yl boronic acid (56.27 mg), 1,4-dioxane (2 mL), potassium carbonate (120.53 mg), water (0.4 mL), and [1,1′-bis(diphenylphosphino)ferrocene]palladium(II)dichloride is (23.68 mg) were added to a reaction flask and stirred at 60° C. for 1 hour under nitrogen protection, and the reaction solution was concentrated under reduced pressure, followed by column chromatography purification (mobile phase: methanol/dichloromethane=3/50 (V/V))) to obtain 50 mg of the title compound.

### f) Preparation of 2-cyclopropyl-9-[4-(difluoromethoxy)phenyl]-7-(2-methyl-2H-indazol-5-yl)-8H-pyrido[1,2-a]pyrimidin-8-one

The 9-bromo-2-cyclopropyl-7-(2-methyl-2H-indazol-5-yl)-8H-pyrido[1,2-a]pyrimidin-8-one (40 mg), 4-(difluoromethoxy)phenyl boronic acid (38.04 mg), 1,4-dioxane (1 mL), water (0.2 mL), potassium carbonate (41.96 mg), and [1,1′-bis(diphenylphosphino)ferrocene]palladium(II)dichloride (7.40 mg) were added to a reaction flask and stirred at 80° C. for 1 hour under nitrogen protection, and the reaction solution was concentrated under reduced pressure to prepare and separate 15.4 mg of the title compound. Preparation and separation conditions: (chromatographic column: XBridge Prep OBD C18, 30*150 mm, 5 μm; mobile phase A: water (10 mmol/L ammonium formate), mobile phase B: acetonitrile; flow rate: 60 mL/min; gradient: 35% B to 75% B in 8 min, 75% B; test wavelength: 220 nm; retention time (min): 7.05; column temperature: 25° C.).

¹H NMR (400 MHz, DMSO-d₆) δ 8.52-8.44 (m, 2H), 8.41 (s, 1H), 8.13 (t, J=1.3 Hz, 1H), 7.61-7.56 (m, 2H), 7.52-7.43 (m, 2H), 7.29 (t, J=72 Hz, 1H), 7.20-7.08 (m, 2H), 6.82 (d, J=7.2 Hz, 1H), 4.19 (s, 3H), 2.05 (ddd, J=16.9, 8.3, 4.2 Hz, 1H), 1.05 (dq, J=6.7, 3.7, 3.2 Hz, 2H), 0.93 (dq, J=7.4, 4.1, 3.6 Hz, 2H). LCMS m/z=459[M+1]⁺.

### Example 26: 2-cyclopropyl-9-(2,2-difluoro-1,3-benzodioxol-5-yl)-7-(2-methyl-2H-indazol-5-yl)-8H-pyrido[1,2-a]pyrimidin-8-one

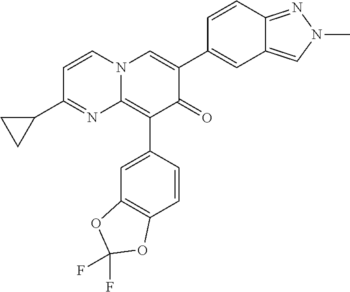 [SMILES: Cn1cc2cc(-c3cn4ccc(C5CC5)nc4c(-c4ccc5c(c4)OC(F)(F)O5)c3=O)ccc2n1]

With reference to the preparation method of Example 25, the 4-(difluoromethoxy)phenyl boronic acid in step f) was replaced with 2,2-difluoro-1,3-benzodioxol-5-yl boronic acid.

¹H NMR (400 MHz, DMSO-d₆) δ 8.49-8.44 (m, 2H), 8.41 (s, 1H), 8.17-8.12 (m, 1H), 7.65-7.59 (m, 1H), 7.56 (dd, J=9.0, 1.6 Hz, 1H), 7.41 (d, J=1.6 Hz, 1H), 7.37 (d, J=8.4 Hz, 1H), 7.25 (dd, J=8.4, 1.6 Hz, 1H), 6.85 (d, J=7.2 Hz, 1H), 4.19 (s, 3H), 2.06 (dt, J=8.0, 3.6 Hz, 1H), 1.11-1.02 (m, 2H), 0.94 (dt, J=4.5, 3.2 Hz, 2H). LCMS m/z=473.05[M+1]⁺.

### Example 27: 2-cyclopropyl-9-(2,2-difluoro-1,3-benzodioxol-5-yl)-7-[4-(difluoromethoxy)phenyl]-8H-pyrido[1,2-a]pyrimidin-8-one

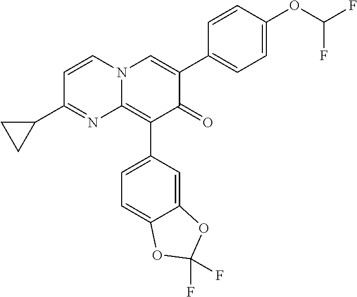 [SMILES: O=c1c(-c2ccc(OC(F)F)cc2)cn2ccc(C3CC3)nc2c1-c1ccc2c(c1)OC(F)(F)O2]

With reference to the preparation method of Example 25, the 2-methyl-2H-indazol-5-yl boronic acid in step e) was replaced with 4-(difluoromethoxy)phenyl boronic acid, and the 4-(difluoromethoxy)phenyl boronic acid in step f) was replaced with 2,2-difluoro-1,3-benzodioxol-5-yl boronic acid.

¹H NMR (400 MHz, DMSO-d₆) δ 8.52-8.31 (m, 2H), 7.86-7.68 (m, 2H), 7.42-7.32 (m, 2H), 7.30 (t, J=72.0 Hz, 1H), 7.28-7.17 (m, 3H), 6.86 (d, J=7.2 Hz, 1H), 2.06 (dp, J=12.5, 4.5, 4.0 Hz, 1H), 1.06 (dq, J=7.0, 3.7 Hz, 2H), 0.93 (t, J=3.8 Hz, 2H). LCMS m/z=485 [M+1]⁺.

### Example 28: 7,9-bis(2H-1,3-benzodioxol-5-yl)-2-cyclopropyl-8H-pyrido[1,2-a]pyrimidin-8-one

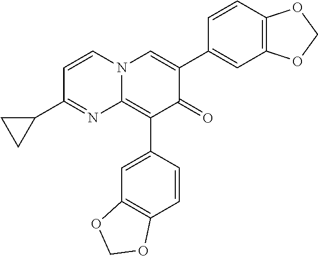 [SMILES: O=c1c(-c2ccc3c(c2)OCO3)cn2ccc(C3CC3)nc2c1-c1ccc2c(c1)OCO2]

With reference to the preparation method of Example 25, the 2-methyl-2H-indazol-5-yl boronic acid and the 4-(difluoromethoxy)phenyl boronic acid in steps e) and f) were replaced with 2H-1,3-benzodioxol-5-yl boronic acid.

¹H NMR (400 MHz, DMSO-d₆) δ 8.36 (d, J=7.3 Hz, 1H), 8.33 (s, 1H), 7.34 (d, J=1.7 Hz, 1H), 7.21 (dd, J=8.1, 1.7 Hz, 1H), 6.98 (d, J=8.1 Hz, 1H), 6.91-6.88 (m, 1H), 6.8 8-6.82 (m, 2H), 6.78 (d, J=7.2 Hz, 1H), 6.05 (s, 2H), 6.01 (s, 2H), 2.06-1.97 (m, 1H), 1.07-0.99 (m, 2H), 0.95-0.87 (m, 2H). LCMS m/z=427 [M+1]⁺.

### Example 29: 2-cyclopropyl-7,9-bis(2-methyl-2H-indazol-5-yl)-8H-pyrido[1,2-a]pyrimidin-8-one

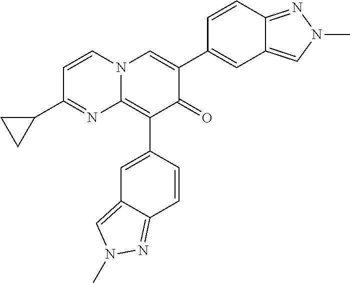 [SMILES: Cn1cc2cc(-c3cn4ccc(C5CC5)nc4c(-c4ccc5nn(C)cc5c4)c3=O)ccc2n1]

With reference to the preparation method of Example 25, the 2-methyl-2H-indazol-5-yl boronic acid and the 4-(difluoromethoxy)phenyl boronic acid in steps e) and f) were replaced with 2-methyl-2H-indazol-5-yl boronic acid.

¹H NMR (400 MHz, DMSO-d₆) δ 8.48-8.38 (m, 3H), 8.28 (s, 1H), 8.15 (s, 1H), 7.69 (s, 1H), 7.64-7.54 (m, 2H), 7.49 (d, J=8.9 Hz, 1H), 7.24 (dd, J=9.0, 1.5 Hz, 1H), 6.81 (d, J=7.2 Hz, 1H), 4.18 (d, J=5.0 Hz, 6H), 2.01 (d, J=4.5 Hz, 1H), 1.00 (dd, J=7.7, 3.6 Hz, 2H), 0.88 (d, J=4.3 Hz, 2H). LCMS m/z=447 [M+1]⁺.

### Example 30: 2-cyclopropyl-7,9-bis(2,2-difluoro-1,3-benzodioxol-5-yl)-8H-pyrido[1,2-a]pyrimidin-8-one

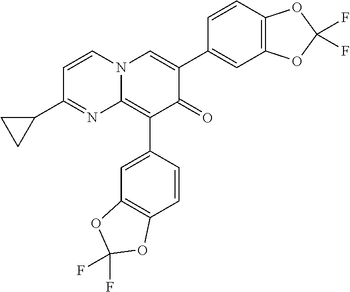 [SMILES: O=c1c(-c2ccc3c(c2)OC(F)(F)O3)cn2ccc(C3CC3)nc2c1-c1ccc2c(c1)OC(F)(F)O2]

With reference to the preparation method of Example 25, the 2-methyl-2H-indazol-5-yl boronic acid and the 4-(difluoromethoxy)phenyl boronic acid in steps e) and f) were replaced with 2,2-difluoro-1,3-benzodioxol-5-yl boronic acid.

¹H NMR (400 MHz, DMSO-d₆) δ 8.49 (s, 1H), 8.41 (d, J=7.2 Hz, 1H), 7.80 (d, J=1.6 Hz, 1H), 7.59-7.47 (m, 2H), 7.41-7.33 (m, 2H), 7.22 (dd, J=8.4, 1.6 Hz, 1H), 6.88 (d, J=7.1 Hz, 1H), 2.06 (qd, J=8.2, 7.3, 4.4 Hz, 1H), 1.07 (dq, J=7.1, 3.7 Hz, 2H), 0.93 (p, J=3.8 Hz, 2H). LCMS m/z=499 [M+1]⁺.

### Example 31: 2-cyclopropyl-7,9-bis(3,4-dimethoxyphenyl)-8H-pyrido[1,2-a]pyrimidin-8-one

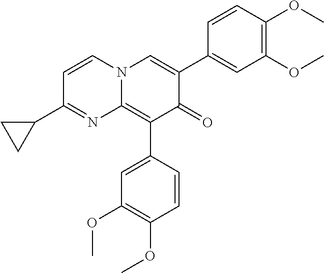 [SMILES: COc1ccc(-c2cn3ccc(C4CC4)nc3c(-c3ccc(OC)c(OC)c3)c2=O)cc1OC]

With reference to the preparation method of Example 25, the 2-methyl-2H-indazol-5-yl boronic acid and the 4-(difluoromethoxy)phenyl boronic acid in steps e) and f) were replaced with 3,4-dimethoxyphenyl boronic acid.

¹H NMR (400 MHz, DMSO-d₆) δ 8.38 (d, J=7.3 Hz, 1H), 8.34 (s, 1H), 7.43 (d, J=2.0 Hz, 1H), 7.29 (dd, J=8.4, 2.0 Hz, 1H), 7.01 (d, J=8.5 Hz, 1H), 6.98-6.90 (m, 3H), 6.79 (d, J=7.2 Hz, 1H), 3.84-3.76 (m, 9H), 3.71 (s, 3H), 2.04 (m, 1H), 1.06-0.98 (m, 2H), 0.92 (dd, J=4.9, 2.7 Hz, 2H).

LCMS m/z=459 [M+1]⁺.

### Example 32: 7-(2H-1,3-benzodioxol-5-yl)-2-cyclopropyl-9-(2,2-difluoro-1,3-benzodioxol-5-yl)pyrido[1,2-a]pyrimidin-8-one

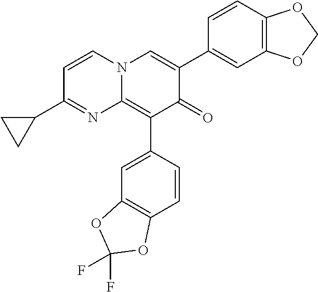 [SMILES: O=c1c(-c2ccc3c(c2)OCO3)cn2ccc(C3CC3)nc2c1-c1ccc2c(c1)OC(F)(F)O2]

With reference to the preparation method of Example 25, the 2-methyl-2H-indazol-5-yl boronic acid in step e) was replaced with 2H-1,3-benzodioxol-5-yl boronic acid, and the 4-(difluoromethoxy)phenyl boronic acid in step f) was replaced with (2,2-difluoro-2H-1,3-benzodioxol-5-yl) boronic acid.

¹H NMR (400 MHz, DMSO-d₆) δ 8.44-8.35 (m, 2H), 7.41-7.31 (m, 3H), 7.22 (td, J=8.1, 1.7 Hz, 2H), 6.99 (d, J=8.1 Hz, 1H), 6.84 (d, J=7.2 Hz, 1H), 6.05 (s, 2H), 2.08-2.00 (m, 1H), 1.09-1.01 (m, 2H), 0.95-0.88 (m, 2H).

LCMS m/z=463 [M+1]⁺

### Example 33: 2-cyclopropyl-9-(2,2-difluoro-1,3-benzodioxol-5-yl)-7-(3,4-dimethoxyphenyl)-8H-pyrido[1,2-a]pyrimidin-8-one

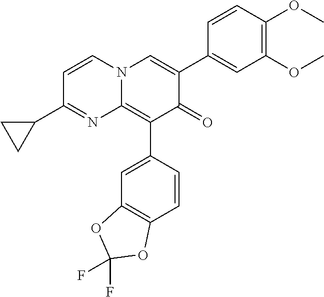 [SMILES: COc1ccc(-c2cn3ccc(C4CC4)nc3c(-c3ccc4c(c3)OC(F)(F)O4)c2=O)cc1OC]

With reference to the preparation method of Example 25, the 2-methyl-2H-indazol-5-yl boronic acid in step e) was replaced with 3,4-dimethoxyphenyl boronic acid, and the 4-(difluoromethoxy)phenyl boronic acid in step f) was replaced with 2,2-difluoro-1,3-benzodioxol-5-yl boronic acid.

¹H NMR (400 MHz, DMSO-d₆) δ 8.43 (d, J=7.3 Hz, 1H), 8.39 (s, 1H), 7.45 (d, J=2.1 Hz, 1H), 7.39-7.33 (m, 2H), 7.30 (dd, J=8.3, 2.0 Hz, 1H), 7.21 (dd, J=8.4, 1.6 Hz, 1H), 7.02 (d, J=8.4 Hz, 1H), 6.84 (d, J=7.2 Hz, 1H), 3.78 (d, J=8.1 Hz, 6H), 2.04 (dt, J=8.0, 3.7 Hz, 1H), 1.11-1.01 (m, 2H), 0.92 (dt, J=4.5, 3.1 Hz, 2H). LCMS m/z=479 [M+1]⁺.

### Example 34: 2-cyclopropyl-9-(2,2-difluoro-1,3-benzodioxol-5-yl)-7-[4-(difluoromethoxy)-3-methoxyphenyl]-8H-pyrido[1,2-a]pyrimidin-8-one

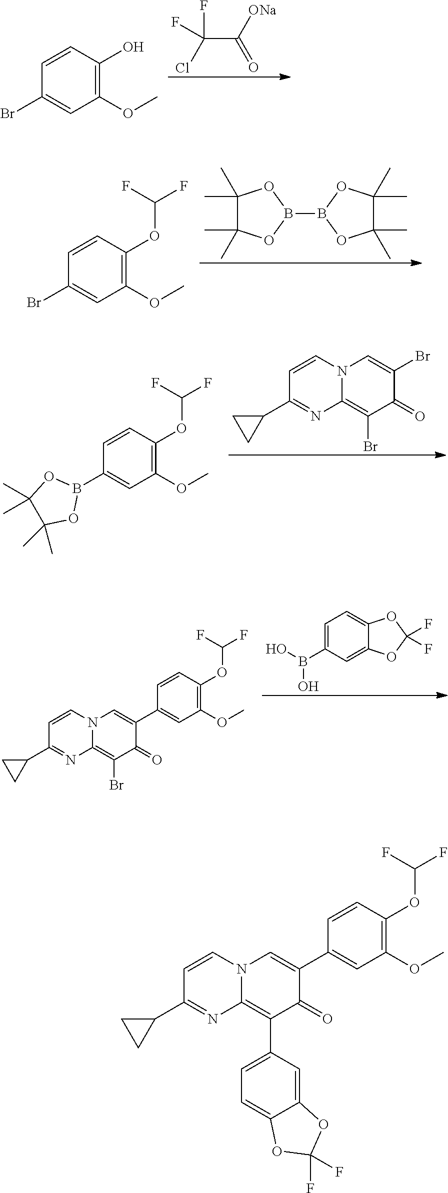

### a) Preparation of 4-bromo-1-(difluoromethoxy)-2-methoxybenzene

4-bromo-2-methoxyphenol (2 g), sodium 2-chloro-2,2-difluoroacetate (1.65 g), and N,N-dimethylformamide (30 mL) were added to a reaction flask, then potassium carbonate (1.63 g) was added under nitrogen protection, the mixture was stirred at 90° C. for 23 hours, the obtained reaction solution was filtered, and the filter cake was washed with ethyl acetate (2×200 mL). The filtrate was concentrated under reduced pressure. Column chromatography purification (mobile phase: ethyl acetate/petroleum ether=3/10 (V/V)) was performed to obtain 500 mg of the title compound.

### b) Preparation of 2-[4-(difluoromethoxy)-3-methoxyphenyl]-4,4,5,5-tetramethyl-1,3,2-dioxaborolane

The 4-bromo-1-(difluoromethoxy)-2-methoxybenzene (200 mg), bis(pinacolato)diboron (301.06 mg), and 1,4-dioxane (4 mL) were added to a reaction flask, potassium acetate (232.71 mg) was added under nitrogen protection, the mixture was stirred at 80° C. for 3 hours, and the reaction solution was concentrated under reduced pressure. Column chromatography purification (mobile phase: ethyl acetate/petroleum ether=1/10 (V/V)) was performed to obtain 200 mg of the title compound.

### c) Preparation of 9-bromo-2-cyclopropyl-7-[4-(difluoromethoxy)-3-methoxyphenyl]-8H-pyrido[1,2-a]pyrimidin-8-one

With reference to the preparation method of Example 25, the 2-methyl-2H-indazol-5-yl boronic acid in step e) was replaced with the 2-[4-(difluoromethoxy)-3-methoxyphenyl]-4,4,5,5-tetramethyl-1,3,2-dioxaborolane.

### d) Preparation of 2-cyclopropyl-9-(2,2-difluoro-1,3-benzodioxol-5-yl)-7-[4-(difluoromethoxy)-3-methoxyphenyl]-8H-pyrido[1,2-a]pyrimidin-8-one

With reference to the preparation method of Example 25, the 4-(difluoromethoxy)phenyl boronic acid in step f) was replaced with 2,2-difluoro-1,3-benzodioxol-5-yl boronic acid.

¹H NMR (400 MHz, DMSO-d₆) δ 8.46 (s, 1H), 8.42 (d, J=7.3 Hz, 1H), 7.60 (d, J=2.0 Hz, 1H), 7.40-7.35 (m, 2H), 7.30 (d, J=1.4 Hz, 1H), 7.26-7.20 (m, 2H), 7.12 (t, J=74 Hz, 1H), 6.87 (d, J=7.2 Hz, 1H), 3.85 (s, 3H), 2.06 (tt, J=8.1, 4.6 Hz, 1H), 1.06 (dq, J=6.9, 3.7 Hz, 2H), 0.93 (p, J=3.7 Hz, 2H). LCMS m/z=515 [M+1]⁺.

### Example 35: 9′-(2-cyclopropyl-9-(4-(difluoromethoxy)phenyl)-8-oxo-8H-pyrido[1,2-a]pyrimidin-7-yl)-4′H-spiro[cyclopropane-1,3′-pyrazino[1,2-b]indazol]-1′(2′H)-one

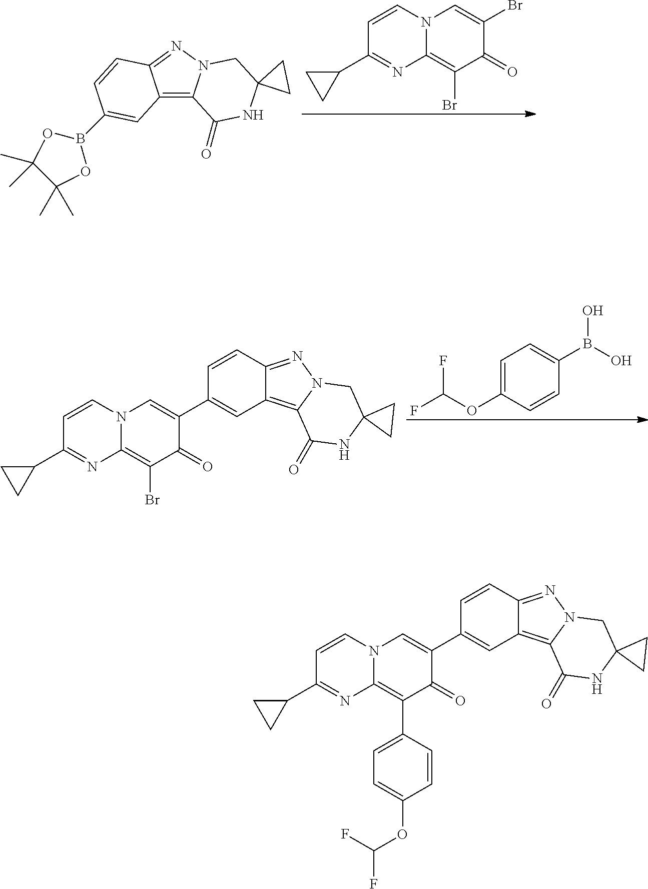

### a) Preparation of 9′-(9-bromo-2-cyclopropyl-8-oxo-8H-pyrido[1,2-a]pyrimidin-7-yl)-4′H-spiro[cyclopropane-1,3′-pyrazino[1,2-b]indazol]-1′(2′H)-one

Under nitrogen protection, 9′-(4,4,5,5-tetramethyl-1,3,2-dioxaborolan-2-yl)-4′H-spiro[cyclopropane-1,3′-pyrazino[1,2-b]indazol]-1′(2′H)-one (60 mg), 7,9-dibromo-2-cyclopropyl-8H-pyri do[1,2-a]pyrimidin-8-one (60.9 mg), and potassium carbonate (73.3 mg) were mixed with 1,4-dioxane (2 mL) and water (0.4 mL), [1,1′-bis(diphenylphosphino)ferrocene]palladium(II)dichloride dichloromethane complex (9.8 mg) was added, and the mixture was heated to 60° C. and stirred for 2 hours. After the reaction was completed, the reaction solution was concentrated to dryness, and the crude product was purified by column chromatography (mobile phase: methanol/dichloromethane=0-1/24 (V/V)) to obtain 50 mg of the title compound.

MS(ESI+): 477.85 (M+H).

### b) Preparation of 9′-(2-cyclopropyl-9-(4-(difluoromethoxy)phenyl)-8-oxo-8H-pyrido[1,2-a]pyrimidin-7-yl)-4′H-spiro[cyclopropane-1,3′-pyrazino[1,2-b]indazol]-1′(2′H)-one

Under nitrogen protection, the 9′-(9-bromo-2-cyclopropyl-8-oxo-8H-pyrido[1,2-a]pyrimidin-7-yl)-4′H-spiro[cyclopropane-1,3′-pyrazino[1,2-b]indazol]-1′(2′H)-one (60 mg), 4-(difluoromethoxy)phenyl boronic acid (47.4 mg), [1,1′-bis(diphenylphosphino)ferrocene]palladium(II)dichloride dichloromethane complex (10.3 mg), and potassium carbonate (52.23 mg) were mixed with 1,4-dioxane (2 mL) and water (0.4 mL), and the mixture was heated to 80° C. and stirred for 2 hours. After the reaction was completed, the reaction solution was concentrated to dryness, and the crude product was purified to obtain 14.9 mg of the title compound by chromatography (chromatographic column: XBridge Prep OBD C18, 30*150 mm, 5 μm; mobile phase A: water (10 mmol/L ammonium formate), mobile phase B: acetonitrile; flow rate: 60 mL/min; gradient: 20% B to 60% B in 8 min, 60% B; test wavelength: 220 nm; retention time (min): 7.37; column temperature: 25° C.).

¹H NMR (400 MHz, DMSO-d6): δ 8.60-8.40 (m, 4H), 7.81 (dd, J=9.0, 0.9 Hz, 1H), 7.72 (dd, J=9.0, 1.7 Hz, 1H), 7.52-7.48 (m, 2H), 7.29 (t, J=74 Hz 1H), 7.19-7.12 (m, 2H), 6.84 (d, J=7.2 Hz, 1H), 4.65 (s, 2H), 2.05 (ddd, J=12.7, 8.4, 4.8 Hz, 1H), 1.09-1.03 (m, 2H), 0.99 (d, J=4.3 Hz, 2H), 0.94 (ddd, J=6.5, 4.7, 2.4 Hz, 4H). MS(ESI+): 540 (M+H).

### Example 36: 9′-(2-cyclopropyl-9-(4-(difluoromethoxy)phenyl)-8-oxo-8H-pyrido[1,2-a]pyrimidin-7-yl)-2′-methyl-4′H-spiro[cyclopropane-1,3′-pyrazino[1,2-b]indazol]-1′(2′H)-one

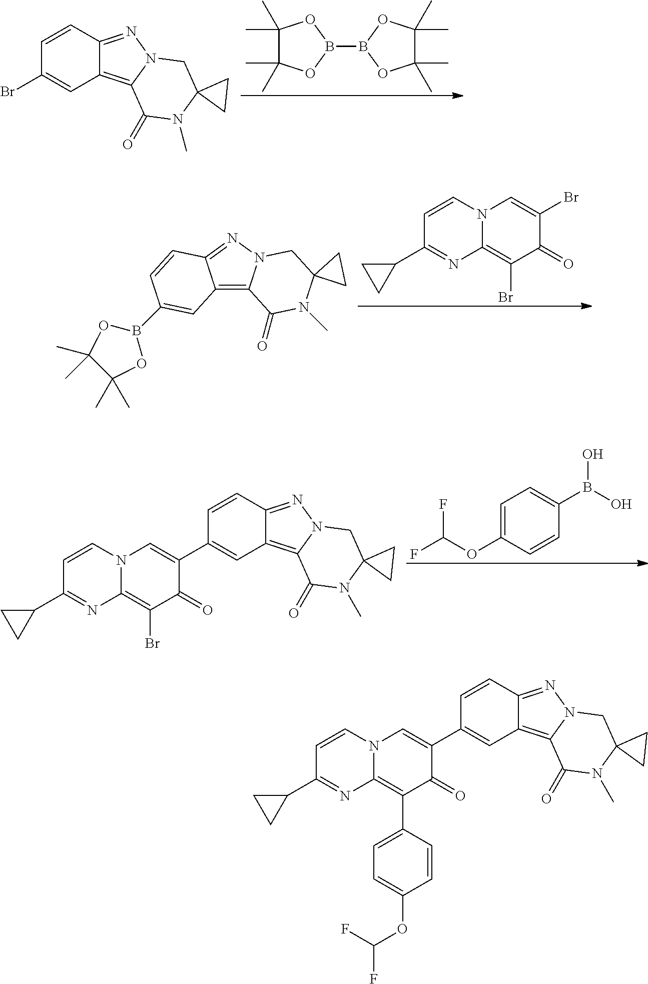

### a) Preparation of 2′-methyl-9′-(4,4,5,5-tetramethyl-1,3,2-dioxaborolan-2-yl)-4′H-spiro[cyclopropane-1,3′-pyrazino[1,2-b]indazol]-1′(2′H)-one

Under nitrogen protection, 9′-bromo-2′-methyl-4′H-spiro[cyclopropane-1,3′-pyrazino[1,2-b]indazol]-1′(2′H)-one (100 mg), bis(pinacolato)diboron (99.5 mg), and potassium acetate (96.2 mg) were mixed with 1,4-dioxane (10 mL), [1,1′-bis(diphenylphosphino)ferrocene]palladium(II)dichloride dichloromethane complex (13.3 mg) was added, and the mixture was heated to 80° C. and stirred for 2 hours. After the reaction was completed, the reaction solution was concentrated to dryness, and the crude product was purified by column chromatography (mobile phase: ethyl acetate/petroleum ether=0-1/1 (V/V)) to obtain 60 mg of the title compound.

MS(ESI+): 353.95 (M+H).

### b) Preparation of 9′-(9-bromo-2-cyclopropyl-8-oxo-8H-pyrido[1,2-a]pyrimidin-7-yl)-2′-methyl-4′H-spiro[cyclopropane-1,3′-pyrazino[1,2-b]indazol]-1′(2′H)-one

Under nitrogen protection, the 2′-methyl-9′-(4,4,5,5-tetramethyl-1,3,2-dioxaborolan-2-yl)-4′H-spiro[cyclopropane-1,3′-pyrazino[1,2-b]indazol]-1′(2′H)-one (50 mg), 7,9-dibromo-2-cyclopropyl-8H-pyrido[1,2-a]pyrimidin-8-one (48.7 mg), and potassium carbonate (58.7 mg) were mixed with 1,4-dioxane (5 mL) and water (1 mL), [1,1′-bis(diphenylphosphino)ferrocene]palladium(II)dichloride dichloromethane complex (11.5 mg) was added, and the mixture was heated to 60° C. and stirred for 2 hours. After the reaction was completed, the reaction solution was concentrated to dryness, and the crude product was purified by column chromatography (mobile phase: methanol/dichloromethane=0-1/24 (V/V)) to obtain 50 mg of the title compound.

MS(ESI+): 491.90 (M+H).

### c) Preparation of 9′-(2-cyclopropyl-9-(4-(difluoromethoxy)phenyl)-8-oxo-8H-pyrido[1,2-a]pyrimidin-7-yl)-2′-methyl-4′H-spiro[cyclopropane-1,3′-pyrazino[1,2-b]indazol]-1′(2′H)-one

Under nitrogen protection, the 9′-(9-bromo-2-cyclopropyl-8-oxo-8H-pyrido[1,2-a]pyrimidin-7-yl)-2′-methyl-4′H-spiro[cyclopropane-1,3′-pyrazino[1,2-b]indazol]-1′(2′H)-one (50 mg), 4-(difluoromethoxy)phenyl boronic acid (38.3 mg), [1,1′-bis(diphenylphosphino)ferrocene]palladium(II)dichloride dichloromethane complex (8.3 mg), and potassium carbonate (42.3 mg) were mixed with 1,4-dioxane (2 mL) and water (0.4 mL), and the mixture was heated to 80° C. and stirred for 2 hours. After the reaction was completed, the reaction solution was concentrated to dryness, and the crude product was purified to obtain 12.2 mg of the title compound by chromatography (chromatographic column: YMC-Actus Triart C18, 30*150 mm, 5 μm; mobile phase A: water (10 mmol/L ammonium formate), mobile phase B: acetonitrile; flow rate: 60 mL/min; gradient: 20% B to 67% B in 8 min, 67% B; test wavelength: 220 nm; retention time (min): 8.15; column temperature: 25° C.).

¹H NMR (400 MHz, DMSO-d6): δ 8.52-8.39 (m, 3H), 7.80 (dd, J=9.0, 0.9 Hz, 1H), 7.71 (dd, J=9.0, 1.7 Hz, 1H), 7.52-7.48 (m, 2H), 7.29 (t, J=74 Hz, 1H), 7.19-7.13 (m, 2H), 6.84 (d, J=7.2 Hz, 1H), 4.67 (s, 2H), 2.90 (s, 3H), 2.05 (t, J=4.4 Hz, 1H), 1.33-1.22 (m, 2H), 1.09-1.02 (m, 2H), 1.00 (d, J=6.5 Hz, 2H), 0.94 (dd, J=5.2, 2.6 Hz, 2H). MS(ESI+): 554 (M+H).

### Example 37: 2-cyclopropyl-7,9-bis(4-(difluoromethoxy)-3-methoxyphenyl)-8H-pyrido[1,2-a]pyrimidin-8-one

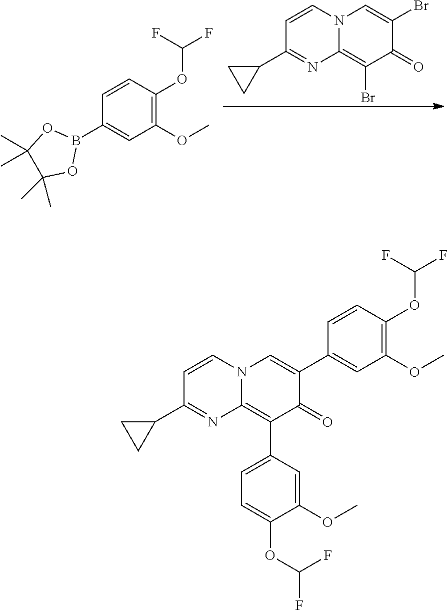

### a) Preparation of 2-cyclopropyl-7,9-bis(4-(difluoromethoxy)-3-methoxyphenyl)-8H-pyrido[1,2-a]pyrimidin-8-one

Under nitrogen protection, 2-(4-(difluoromethoxy)-3-methoxyphenyl)-4,4,5,5-tetramethyl-1,3,2-dioxaborolane (50 mg), 7,9-dibromo-2-cyclopropyl-8H-pyrido[1,2-a]pyrimidin-8-one (28.7 mg), [1,1′-bis(diphenylphosphino)ferrocene]palladium(II)dichloride dichloromethane complex (13.6 mg), and potassium carbonate (69.1 mg) were mixed with 1,4-dioxane (1 mL) and water (0.2 mL), and the mixture was heated to 80° C. and stirred for 1 hour. After the reaction was completed, the reaction solution was concentrated to dryness, and the crude product was purified to obtain 13.8 mg of the title compound by chromatography (chromatographic column: XBridge Prep OBD C18, 30*150 mm, 5 μm; mobile phase A: water (10 mmol/L ammonium formate), mobile phase B: acetonitrile; flow rate: 60 mL/min; gradient: 30% B to 65% B in 8 min, 65% B; test wavelength: 220 nm; retention time (min): 9.28; column temperature: 25° C.).

¹H NMR (400 MHz, DMSO-d6): δ 8.44 (s, 1H), 8.40 (d, J=7.2 Hz, 1H), 7.59 (d, J=1.9 Hz, 1H), 7.31 (dd, J=8.5, 2.1 Hz, 1H), 7.25 (s, 1H), 7.16 (d, J=1.9 Hz, 1H), 7.13 (t, J=72 Hz, 1H), 7.11 (d, J=2.0 Hz, 1H), 7.09 (t, J=64 Hz, 1H), 6.99 (dd, J=8.3, 1.9 Hz, 1H), 6.85 (d, J=7.2 Hz, 1H), 3.85 (s, 3H), 3.79 (s, 3H), 2.06 (tt, J=8.2, 4.5 Hz, 1H), 1.09-1.02 (m, 2H), 0.94 (dt, J=4.5, 3.2 Hz, 2H). MS(ESI+): 530 (M+H).

### Example 38: 2-cyclopropyl-9-(4-(difluoromethoxy)phenyl)-7-(2′-methyl-1′,2′-dihydro-4′H-spiro[cyclopropane-1,3′-pyrazino[1,2-b]indazol]-9′-yl)-8H-pyrido[1,2-a]pyrimidin-8-one

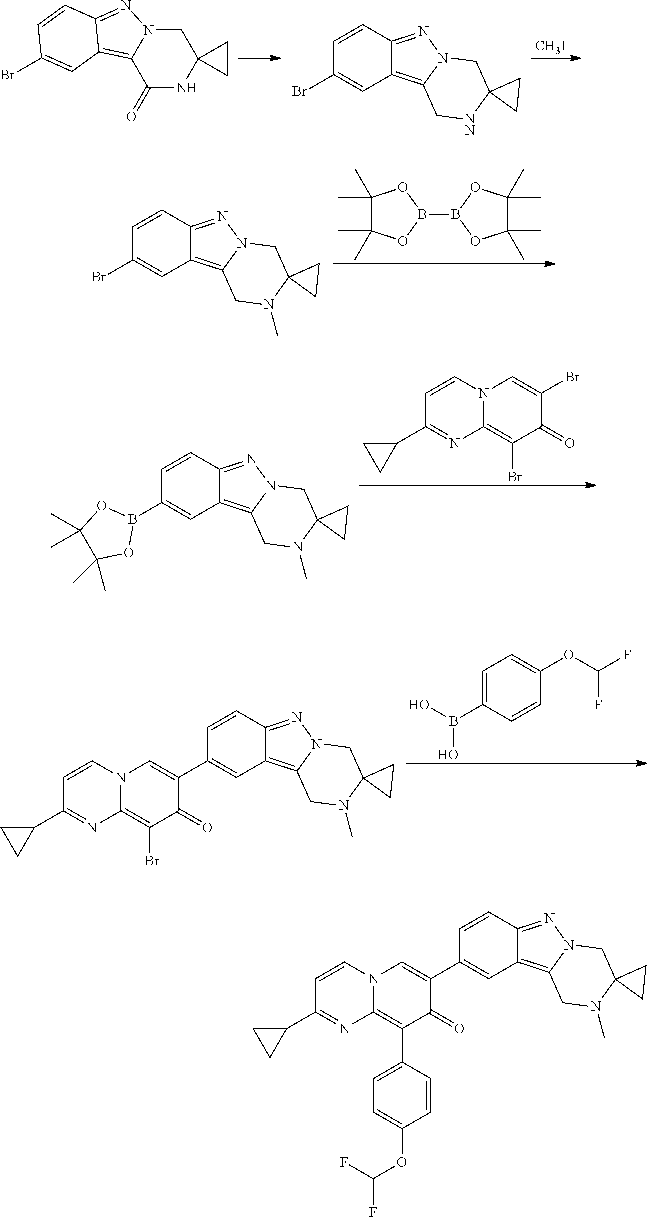

### a) Preparation of 9′-bromo-1′,2′-dihydro-4′H-spiro[cyclopropane-1,3′-pyrazino[1,2-b]indazole]

9′-bromo-4′H-spiro[cyclopropane-1,3′-pyrazino[1,2-b]indazol]-1′(2′H)-one (100 mg) was mixed with tetrahydrofuran (2 mL), a borane tetrahydrofuran solution (1 M, 1.7 mL) was added, and the mixture was heated to 70° C. and stirred for 2 hours. After the reaction was completed, the reaction solution was concentrated to dryness, 20 mL of ether was added, filtration was performed, and the filter cake was washed twice with 20 mL of ether to obtain 25 mg of the title compound.

MS(ESI+): 279.65 (M+H).

### b) Preparation of 9′-bromo-2′-methyl-1′,2′-dihydro-4′H-spiro[cyclopropane-1,3′-pyrazino[1,2-b]indazole]

The 9′-bromo-1′,2′-dihydro-4′H-spiro[cyclopropane-1,3′-pyrazino[1,2-b]indazole] (200 mg) and sodium hydride (60%, 86.3 mg) were mixed with N,N-dimethylformamide (10 mL), iodomethane (408.2 mg) was added in an ice bath, and the mixture was stirred at room temperature for 4 hours. After the reaction was completed, water was added for quenching in the ice bath, and the reaction solution was extracted 3 times with 30 mL of ethyl acetate. Organic phases were combined and washed with 30 mL of saturated salt solution. The organic phase was concentrated to dryness, and the crude product was purified by column chromatography (mobile phase: ethyl acetate/petroleum ether=0-100% (V/V)) to obtain 70 mg of the title compound.

MS(ESI+): 293.85 (M+H).

### c) Preparation of 2′-methyl-9′-(4,4,5,5-tetramethyl-1,3,2-dioxaborolan-2-yl)-1′,2′-dihydro-4′H-spiro[cyclopropane-1,3′-pyrazino[1,2-b]indazole]

Under nitrogen protection, the 9′-bromo-2′-methyl-1′,2′-dihydro-4′H-spiro[cyclopropane-1,3′-pyrazino[1,2-b]indazole] (80 mg), bis(pinacolato)diboron (83.4 mg), and potassium acetate (80.62 mg) were mixed with 1,4-dioxane (8 mL), [1,1′-bis(diphenylphosphino)ferrocene]palladium(II)dichloride dichloromethane complex (22.3 mg) was added, and the mixture was heated to 80° C. and stirred for 1 hour. After the reaction was completed, the reaction solution was concentrated to dryness, and the crude product was purified by column chromatography (mobile phase: ethyl acetate/petroleum ether=0-100% (V/V)) to obtain 70 mg of the title compound.

MS(ESI+): 339.65 (M+H).

### d) Preparation of 9-bromo-2-cyclopropyl-7-(2′-methyl-1′,2′-dihydro-4′H-spiro[cyclopropane-1,3′-pyrazino[1,2-b]indazol]-9′-yl)-8H-pyrido[1,2-a]pyrimidin-8-one

Under nitrogen protection, the 2′-methyl-9′-(4,4,5,5-tetramethyl-1,3,2-dioxaborolan-2-yl)-1′,2′-dihydro-4′H-spiro[cyclopropane-1,3′-pyrazino[1,2-b]indazole] (70 mg), 7,9-dibromo-2-cyclopropyl-8H-pyrido[1,2-a]pyrimidin-8-one (71 mg), and potassium carbonate (85.6 mg) were mixed with 1,4-dioxane (1.75 mL) and water (0.35 mL), [1,1′-bis(diphenylphosphino)ferrocene]palladium(II)dichloride dichloromethane complex (11.5 mg) was added, and the mixture was heated to 60° C. and stirred for 1 hour. After the reaction was completed, the reaction solution was concentrated to dryness, and the crude product was purified by column chromatography (mobile phase: methanol/dichloromethane=0-1/10 (V/V)) to obtain 60 mg of the title compound.

MS(ESI+): 477.90 (M+H).

### e) Preparation of 2-cyclopropyl-9-(4-(difluoromethoxy)phenyl)-7-(2′-methyl-1′,2′-dihydro-4′H-spiro[cyclopropane-1,3′-pyrazino[1,2-b]indazol]-9′-yl)-8H-pyrido[1,2-a]pyrimidin-8-one

Under nitrogen protection, the 9-bromo-2-cyclopropyl-7-(2′-methyl-1′,2′-dihydro-4′H-spiro[cyclopropane-1,3′-pyrazino[1,2-b]indazol]-9′-yl)-8H-pyrido[1,2-a]pyrimidin-8-one (60 mg), 4-(difluoromethoxy)phenyl boronic acid (35.5 mg), [1,1′-bis(diphenylphosphino)ferrocene]palladium(II)dichloride dichloromethane complex (10.3 mg), and potassium carbonate (52.2 mg) were mixed with 1,4-dioxane (2 mL) and water (0.4 mL), and the mixture was heated to 80° C. and stirred for 2 hours. After the reaction was completed, the reaction solution was concentrated to dryness, and the crude product was purified to obtain 15.9 mg of the title compound by chromatography (chromatographic column: XBridge Prep OBD C18, 30*150 mm, 5 μm; mobile phase A: water (10 mmol/L ammonium formate), mobile phase B: acetonitrile; flow rate: 60 mL/min; gradient: 20% B to 58% B in 8 min, 58% B; test wavelength: 220 nm; retention time (min): 7.58; column temperature: 25° C.).

¹H NMR (400 MHz, DMSO-d6): δ 8.46-8.41 (m, 2H), 8.13 (d, J=1.3 Hz, 1H), 7.57 (d, J=1.3 Hz, 2H), 7.48-7.45 (m, 2H), 7.27 (t, J=72 Hz, 1H), 7.17-7.11 (m, 2H), 6.82 (d, J=7.2 Hz, 1H), 4.30 (d, J=17.1 Hz, 4H), 2.39 (s, 3H), 2.04 (tt, J=8.1, 4.7 Hz, 1H), 1.04 (dd, J=7.9, 3.3 Hz, 2H), 0.93 (t, J=3.8 Hz, 2H), 0.82 (t, J=2.9 Hz, 2H), 0.76-0.68 (m, 2H). MS(ESI+): 540 (M+H).

### Example 39: 2-cyclopropyl-9-(4-(difluoromethoxy)phenyl)-7-(1′,2′-dihydro-4′H-spiro[cyclopropane-1,3′-pyrazino[1,2-b]indazol]-9′-yl)-8H-pyrido[1,2-a]pyrimidin-8-one

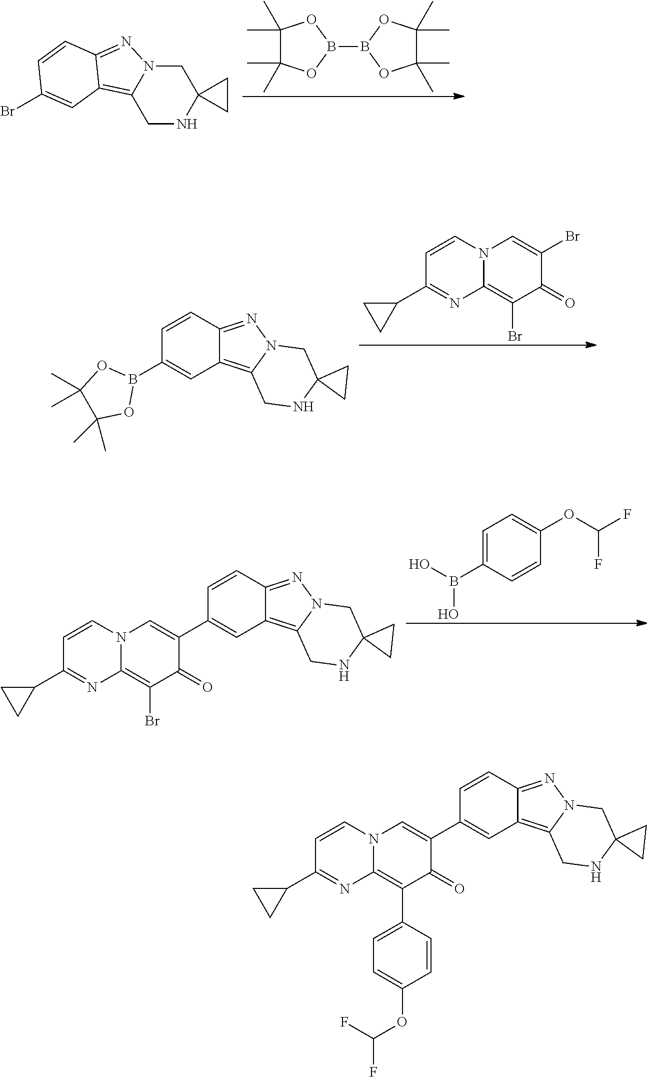

### a) Preparation of 9′-(4,4,5,5-tetramethyl-1,3,2-dioxaborolan-2-yl)-1′,2′-dihydro-4′H-spiro[cyclopropane-1,3′-pyrazino[1,2-b]indazole]

Under nitrogen protection, the 9′-bromo-1′,2′-dihydro-4′H-spiro[cyclopropane-1,3′-pyrazino[1,2-b]indazole] (20 mg), bis(pinacolato)diboron (21.91 mg), and potassium acetate (21.17 mg) were mixed with 1,4-dioxane (2 mL), [1,1′-bis(diphenylphosphino)ferrocene]palladium(II)dichloride dichloromethane complex (2.93 mg) was added, and the mixture was heated to 80° C. and stirred for 2 hours. After the reaction was completed, the reaction solution was concentrated to dryness, and the crude product was purified by column chromatography (mobile phase: methanol/dichloromethane=0-1/10 (V/V)) to obtain 26 mg of the title compound.

MS(ESI+): 326.10 (M+H).

### b) Preparation of 9-bromo-2-cyclopropyl-7-(1′,2′-dihydro-4′H-spiro[cyclopropane-1,3′-pyrazino[1,2-b]indazol]-9′-yl)-8H-pyrido[1,2-a]pyrimidin-8-one

Under nitrogen protection, the 9′-(4,4,5,5-tetramethyl-1,3,2-dioxaborolan-2-yl)-1′,2′-dihydro-4′H-spiro[cyclopropane-1,3′-pyrazino[1,2-b]indazole] (70 mg), 7,9-dibromo-2-cyclopropyl-8H-pyrido[1,2-a]pyrimidin-8-one (74 mg), and potassium carbonate (89.2 mg) were mixed with 1,4-dioxane (2 mL) and water (0.4 mL), [1,1′-bis(diphenylphosphino)ferrocene]palladium(II)dichloride dichloromethane complex (11.9 mg) was added, and the mixture was heated to 60° C. and stirred for 1 hour. After the reaction was completed, the reaction solution was concentrated to dryness, and the crude product was purified by column chromatography (mobile phase: methanol/dichloromethane=0-1/15 (V/V)) to obtain 60 mg of the title compound.

MS(ESI+): 464.00 (M+H).

### c) Preparation of 2-cyclopropyl-9-(4-(difluoromethoxy)phenyl)-7-(1′,2′-dihydro-4′H-spiro[cyclopropane-1,3′-pyrazino[1,2-b]indazol]-9′-yl)-8H-pyrido[1,2-a]pyrimidin-8-one

Under nitrogen protection, the 9-bromo-2-cyclopropyl-7-(1′,2′-dihydro-4′H-spiro[cyclopropane-1,3′-pyrazino[1,2-b]indazol]-9′-yl)-8H-pyrido[1,2-a]pyrimidin-8-one (60 mg), 4-(difluoromethoxy)phenyl boronic acid (36.6 mg), [1,1′-bis(diphenylphosphino)ferrocene]palladium(II)dichloride dichloromethane complex (10.6 mg), and potassium carbonate (53.8 mg) were mixed with 1,4-dioxane (2 mL) and water (0.4 mL), and the mixture was heated to 80° C. and stirred for 2 hours. After the reaction was completed, the reaction solution was concentrated to dryness, and the crude product was purified to obtain 18.5 mg of the title compound by chromatography (chromatographic column: YMC-Actus Triart C18, 30*150 mm, 5 μm; mobile phase A: water (10 mmol/L ammonium formate), mobile phase B: acetonitrile; flow rate: 60 mL/min; gradient: 30% B to 50% B in 10 min, 50% B; test wavelength: 220 nm; retention time (min): 9.28; column temperature: 25° C.).

¹H NMR (400 MHz, DMSO-d6): δ 8.45 (d, J=7.5 Hz, 2H), 8.13 (d, J=1.3 Hz, 1H), 7.56 (d, J=1.2 Hz, 2H), 7.47-7.45 (m, 2H), 7.28 (t, J=74 Hz, 1H), 7.17-7.11 (m, 2H), 6.82 (d, J=7.1 Hz, 1H), 4.26 (s, 4H), 3.21 (s, 2H), 2.03 (dt, J=8.0, 3.6 Hz, 1H), 1.04 (dq, J=6.7, 3.7, 3.2 Hz, 1H), 0.93 (q, J=3.5 Hz, 2H), 0.80-0.72 (m, 2H), 0.69 (q, J=5.1, 4.3 Hz, 2H). MS(ESI+): 526 (M+H).

### Example 40: 4-cyclopropyl-7,9-bis[4-(difluoromethoxy)phenyl]-8H-pyrido[1,2-a]pyrimidin-8-one

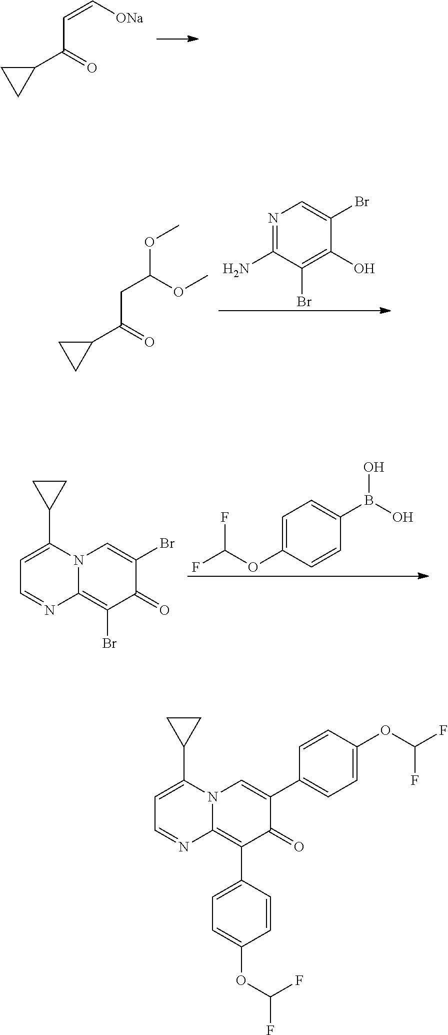

### a) Preparation of 1-cyclopropyl-3,3-dimethoxypropan-1-one

Sodium (Z)-3-cyclopropyl-3-oxoprop-1-en-1-ate (10 g), methanol (100 mL), and concentrated sulfuric acid (4.4 mL) were added to a reaction flask and stirred at 60° C. for 12 hours under nitrogen protection, and the reaction solution was cooled to 0° C. The reaction was quenched with saturated sodium bicarbonate aqueous solution, the reaction solution was extracted with ethyl acetate (3×200 mL), organic layers are combined, washed with saturated salt solution (1×300 mL), dried with anhydrous sodium sulfate, and filtered, and the filtrate was concentrated under reduced pressure to obtain 6 g of the title compound.

### b) Preparation of 7,9-dibromo-4-cyclopropyl-8H-pyrido[1,2-a]pyrimidin-8-one

The 1-cyclopropyl-3,3-dimethoxypropan-1-one (500 mg) and 2-amino-3,5-dibromopyridin-4-ol (564.50 mg) were added to a reaction flask, then a 1,4-dioxane (4 M, 10 mL) solution of hydrogen chloride was added, and the mixture was stirred overnight under room temperature nitrogen protection. The reaction mixture was concentrated under reduced pressure to obtain 720 mg of the title compound.

### c) Preparation of 4-cyclopropyl-7,9-bis[4-(difluoromethoxy)phenyl]-8H-pyrido[1,2-a]pyrimidin-8-one

The 7,9-dibromo-4-cyclopropyl-8H-pyridine[1,2-a]pyrimidin-8-one (500 mg), 1,4-dioxane (10 mL), water (2 mL), 4-(difluoromethoxy)phenyl boronic acid (273.16 mg), potassium carbonate (401.75 mg), and [1,1′-bis(diphenylphosphino)ferrocene]palladium(II)dichloride dichloromethane complex (47.36 mg) were added to a reaction flask and stirred at 80° C. for 2 hours under nitrogen protection, and the reaction solution was concentrated under reduced pressure to prepare and separate 2 mg of the title compound. Preparation and separation conditions: (chromatographic column: XBridge Prep OBD C18, 30*150 mm, 5 μm; mobile phase A: water (10 mmol/L ammonium formate), mobile phase B: acetonitrile; flow rate: 60 mL/min; gradient: 25% B to 65% B in 8 min, 65% B; test wavelength: 220 nm; retention time (min): 7.07; column temperature: 25° C.).

¹H NMR (400 MHz, DMSO-d₆) δ 8.78 (s, 1H), 8.40 (d, J=4.1 Hz, 1H), 7.91-7.78 (m, 2H), 7.42-7.37 (m, 2H), 7.32-7.25 (m, 2H), 7.29 (t, J=74.0 Hz, 1H), 7.31 (t, J=74.0 Hz, 1H), 7.20-7.14 (m, 2H), 6.76 (d, J=4.3 Hz, 1H), 2.49-2.42 (m, 1H), 1.27-1.17 (m, 2H), 1.03-0.95 (m, 2H).

LCMS m/z=471 [M+1]⁺.

### Example 41: 7,9-bis(4-(difluoromethoxy)phenyl)-4-methyl-2-((2,2,2-trifluoroethyl)amino)-8H-pyrido[1,2-a]pyrimidin-8-one

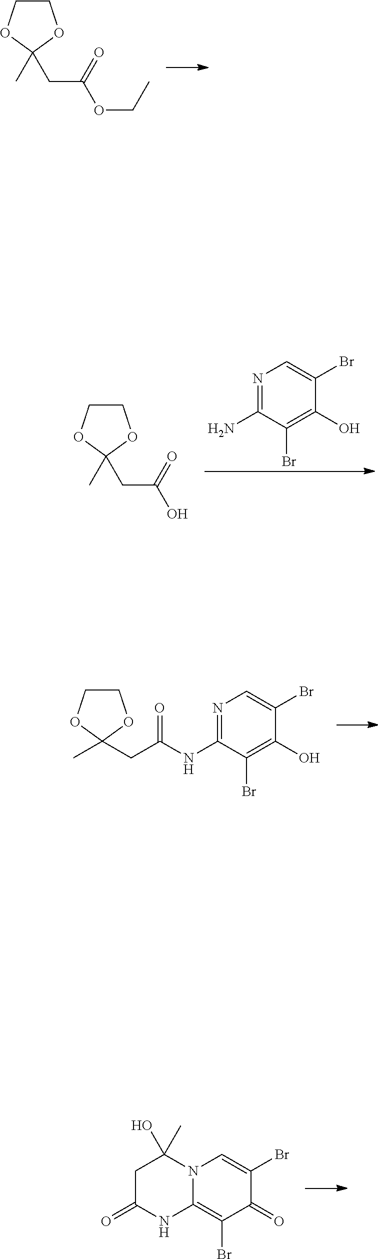

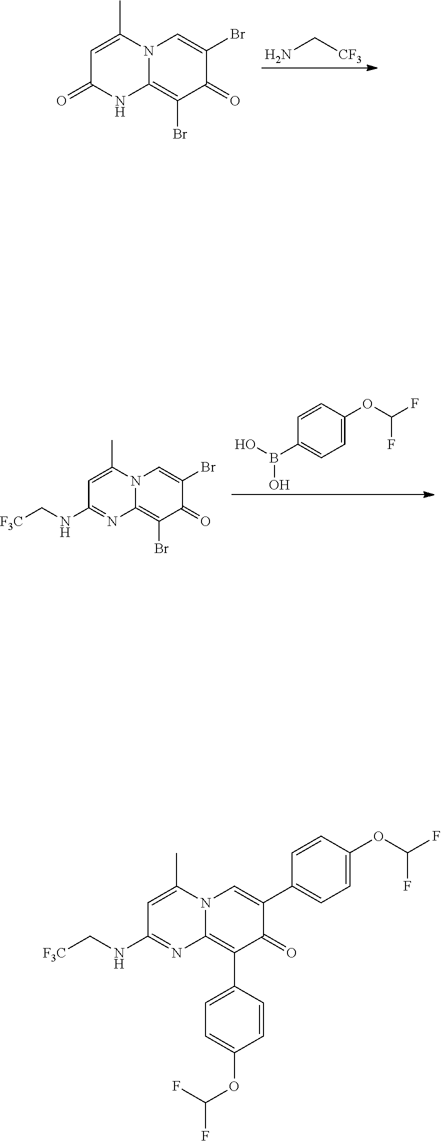 [SMILES: Cc1cc(=O)[nH]c2c(Br)c(=O)c(Br)cn12 ; NCC(F)(F)F ; Cc1cc(NCC(F)(F)F)nc2c(Br)c(=O)c(Br)cn12 ; OB(O)c1ccc(OC(F)F)cc1 ; Cc1cc(NCC(F)(F)F)nc2c(-c3ccc(OC(F)F)cc3)c(=O)c(-c3ccc(OC(F)F)cc3)cn12]

### a) Preparation of 2-(2-methyl-1,3-dioxolan-2-yl) acetic acid

2-(2-methyl-1,3-dioxolan-2-yl)ethyl acetate (16 g), water (80 mL), and ethanol (80 mL) were addedto a flask at room temperature and stirred, then potassium hydroxide (15.46 g) was added, and the mixture was stirred at room temperature for 3 hours under nitrogen protection. After the reaction, the reaction mixture was concentrated to 50 mL and acidified to pH 4 with 1 M HCl (aqueous solution). The obtained mixture was extracted with ethyl acetate (3×200 mL). Organic phases were combined, washed with saturated salt solution (1×100 mL), and dried with anhydrous sodium sulfate. After filtration, the filtrate was concentrated to obtain 12 g of the title compound.

### b) Preparation of N-(3,5-dibromo-4-hydroxypyridin-2-yl)-2-(2-methyl-1,3-dioxolan-2-yl) acetamide

1-hydroxybenzotriazole (1.51 g), 1-(3-dimethylaminopropyl)-3-ethylcarboimide (5.72 g), N,N-diisopropylethylethylamine (5.79 g), and 2-amino-3,5-dibromopyridin-4-ol (1.5 g) were added to an N,N-dimethylformamide (30 mL) solution of the 2-(2-methyl-1,3-dioxolan-2-yl) acetic acid (1.64 g) in stirring. The mixture was stirred at 80° C. for 3 hours under nitrogen protection. After the reaction, the reaction mixture was concentrated. The residue was roughly separated by silica gel column chromatography and eluted with ethyl acetate/petroleum ether (20%-100%) to obtain 700 mg of crude product. Subsequently, the crude product is further purified by silica gel column chromatography (ethyl acetate/petroleum ether-methanol/dichloromethane (20%-100%)-(0-10%)) to obtain 450 mg of the title compound.

### c) Preparation of 7,9-dibromo-4-hydroxy-4-methyl-3,4-dihydro-2H-pyrido[1,2-a]pyrimidin-2,8(1H)-dione

The N-(3,5-dibromo-4-hydroxypyridin-2-yl)-2-(2-methyl-1,3-dioxolan-2-yl) acetamide (1 g) and hydrochloric acid aqueous solution (8 M, 20 mL) were added to a flask and stirred at room temperature for 3 hours. After the reaction, the reaction mixture was concentrated under reduced pressure to 5 mL. The concentrate was filtered and washed with water (1×3 mL) to obtain 500 mg of the title compound.

### d) Preparation of 7,9-dibromo-4-methyl-2H-pyrido[1,2-a]pyrimidin-2,8(1H)-dione

4-methylphenylsulfonic acid (122 mg) was added to a toluene (20 mL) solution of the 7,9-dibromo-4-hydroxy-4-methyl-3,4-dihydro-2H-pyrido[1,2-a]pyrimidin-2,8(1H)-dione (500 mg). The mixture was stirred at 90° C. for 1 hour under nitrogen protection. After the reaction, the reaction mixture was concentrated. The concentrate was pulped with acetonitrile. The pulp was filtered to obtain 120 mg of the title compound.

### e) Preparation of 7,9-dibromo-4-methyl-2-((2,2,2-trifluoroethyl)amino)-8H-pyrido[1,2-a]pyrimidin-8-one

The 7,9-dibromo-4-methyl-2H-pyrido[1,2-a]pyrimidin-2,8(1H)-dione (80 mg), 2,2,2-trifluoroethylamine (48 mg), 2-(7-azobenzotriazole)-N,N,N′,N′-tetramethylurea hexafluorophosphate (137 mg) and N,N-dimethylformamide (3 mL) were added to a flask and stirred. Then N,N-diisopropylethylamine (92.9 mg) was added, and stirring was performed at room temperature for 2 hours. After the reaction, the obtained mixture was washed with water (1×5 mL). Filtration and washing with water (3×5 mL) were performed. 40 mg of the title compound was obtained.

### f) Preparation of 7,9-bis(4-(difluoromethoxy)phenyl)-4-methyl-2-((2,2,2-trifluoroethyl)amino)-8H-pyrido[1,2-a]pyrimidin-8-one

The 7,9-dibromo-4-methyl-2-((2,2,2-trifluoroethyl)amino)-8H-pyrido[1,2-a]pyrimidin-8-one (46 mg), 4-(difluoromethoxy)phenyl boronic acid (63 mg), potassium carbonate (61 mg), [1,1-bis(diphenylphosphino)ferrocene]palladium(II)dichloride (18 mg), water (0.4 mL), and 1,4-dioxane (2 mL) were added to a reaction flask. The mixture was stirred at 80° C. for 2 hours under nitrogen protection. After the reaction, the obtained mixture was concentrated under reduced pressure. The concentrate was purified by preparative HPLC (column: Xselect CSH C18 OBD column 30×150 mm 5 μm, n; mobile phase A: water (10 mmol/L ammonium bicarbonate), mobile phase B: acetonitrile; flow rate: 60 mL/min; gradient: 30% B to 68% B within 8 minutes, 68% B; wavelength: 220 nm; RT1 (min): 7.38; number of operations: 0; column temperature: 25° C.). 22.5 mg of the title compound was obtained.

¹H NMR (400 MHz, DMSO-d₆) δ 8.48 (t, J=6.3 Hz, 1H), 8.03 (s, 1H), 7.84-7.75 (m, 2H), 7.48-7.43 (m, 2H), 7.29 (t, J=74 Hz 1H), 7.23 (d, J=8.2 Hz, 3H), 7.09 (d, J=8.6 Hz, 2H), 6.26 (s, 1H), 4.00 (dq, J=15.9, 9.6, 7.4 Hz, 2H), 2.64 (s, 3H). LCMS m/z=542 [M+1]⁺.

### Example 42: 7,9-bis(4-(difluoromethoxy)phenyl)-3-methyl-2-((2,2,2-trifluoroethyl)amino)-8H-pyrido[1,2-a]pyrimidin-8-one

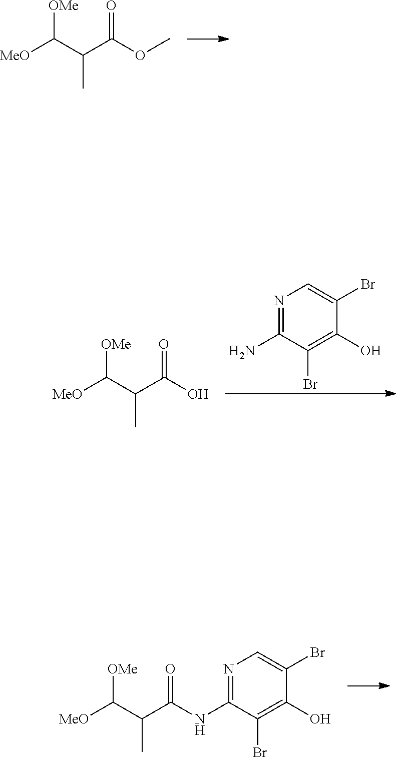

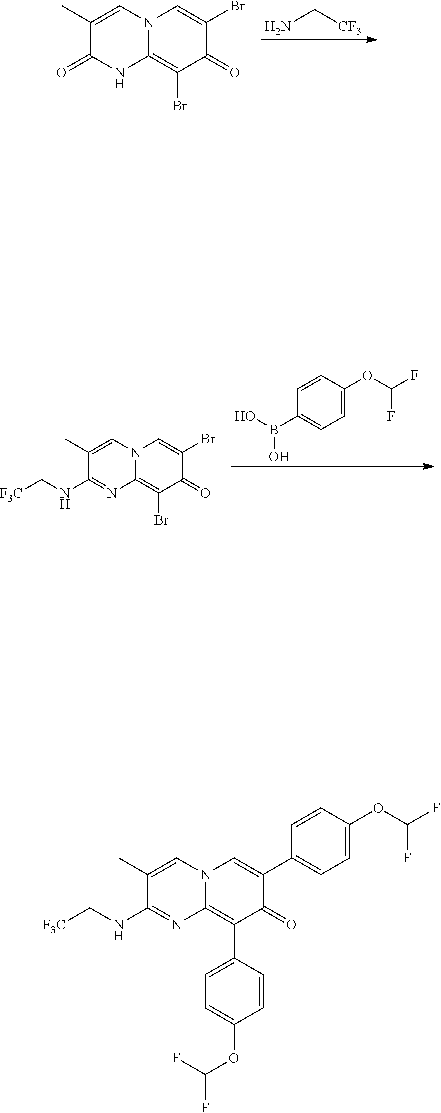 [SMILES: Cc1cn2cc(Br)c(=O)c(Br)c2[nH]c1=O ; NCC(F)(F)F ; Cc1cn2cc(Br)c(=O)c(Br)c2nc1NCC(F)(F)F ; OB(O)c1ccc(OC(F)F)cc1 ; Cc1cn2cc(-c3ccc(OC(F)F)cc3)c(=O)c(-c3ccc(OC(F)F)cc3)c2nc1NCC(F)(F)F]

### a) Preparation of 3,3-dimethoxy-2-methylpropionic acid

3,3-dimethoxy-2-methylpropionate (9.8 g) and water (40 mL) were added to a flask and stirred, and then a water (20 mL) solution of sodium hydroxide (2.9 g) was added at room temperature. The mixture was stirred at 60° C. for 4 hours. After the reaction, the mixture was cooled to room temperature and acidified with 1 M hydrochloric acid aqueous solution to a pH of 3. The obtained mixture was extracted with ethyl acetate (3×80 mL). Organic phases were combined, washed with saturated salt solution (30 mL), and dried with anhydrous sodium sulfate. After filtration, the filtrate was concentrated to obtain 6.9 g of the title compound.

### b) Preparation of N-3,5-dibromo-4-hydroxypyridin-2-yl)-3,3-dimethoxy-2-methylpropanamide

The 3,3-dimethoxy-2-methylpropionic acid (2 g), N,N-diisopropylethylamine (2.62 g), and N,N-dimethylformamide (60 mL) were added to a flask and stirred, followed by batch addition of dicyclohexylcarbodiimide (2.79 g) and 1-hydroxybenzotriazole (1.83 g). The obtained solution was stirred for 10 minutes, and then 2-amino-3,5-dibromopyridin-4-ol (1.81 g) was added. The mixture was stirred at 80° C. for 22 hours. After the reaction, the obtained mixture was filtered, and the filter cake was washed with ethyl acetate (3×15 mL). The filtrate was collected and concentrated to dryness, and the crude product was purified by column chromatography (mobile phase: ethyl acetate/petroleum ether (0%-100%) to obtain 510 mg of the title compound.

### c) Preparation of 7,9-dibromo-3-methyl-2H-pyrido[1,2-a]pyrimidine-2,8(1H)-dione

The N-3,5-dibromo-4-hydroxypyridin-2-yl)-3,3-dimethoxy-2-methylpropanamide (510 mg) and hydrochloric acid aqueous solution (8 M, 15 mL) were added to a flask and stirred at 50° C. for 26 hours. The reaction was monitored by LCMS. After the reaction, the reaction mixture was concentrated to obtain 200 mg of a target compound.

### d) Preparation of 7,9-dibromo-3-methyl-2-((2,2,2-trifluoroethyl)amino)-8H-pyrido[1,2-a]pyrimidin-8-one

The crude product of the 7,9-dibromo-3-methyl-2H-pyrido[1,2-a]pyrimidine-2,8(1H)-dione (200 mg), 2-(7-azobenzotriazole)-N,N,N′,N′-tetramethylurea hexafluorophosphate (342 mg), and N,N-dimethylformamide (6 mL) were added to a flask, and then 2,2,2-trifluoroethylamine (119 mg) and N,N-diisopropylethylamine (232 mg) were added dropwise. The mixture was stirred at room temperature for 4 hours. After the reaction, the obtained mixture was concentrated, and the residue was purified through a reversed-phase chromatographic column (column: C18; mobile phase: acetonitrile/water, gradient from 0% to 50% within 5 minutes; tester: UV 254 nm). 60 mg of the title compound was obtained.

### e) Preparation of 7,9-bis(4-(difluoromethoxy)phenyl)-3-methyl-2-((2,2,2-trifluoroethyl)amino)-8H-pyrido[1,2-a]pyrimidin-8-one

The 7,9-dibromo-3-methyl-2-((2,2,2-trifluoroethyl)amino)-8H-pyrido[1,2-a]pyrimidin-8-one (70 mg), 4-(difluoromethoxy)phenyl boronic acid (63 mg), potassium phosphate (286 mg), [1,1-bis(diphenylphosphino)ferrocene]palladium(II)dichloride (21 mg), water (0.2 mL), and 1,4-dioxane (1 mL) were added to a reaction flask. The mixture was stirred at 80° C. for 2 hours under nitrogen protection. After the reaction, the obtained mixture was concentrated under reduced pressure. The concentrate was purified by preparative HPLC (column: Xselect CSH C18 OBD column 30×150 mm 5 μm, n; mobile phase A: water (10 mmol/L ammonium bicarbonate), mobile phase B: acetonitrile; flow rate: 60 mL/min; gradient: 30% B to 60% B within 8 minutes, 60% B; wavelength: 220 nm; RT1 (min): 9.65; number of operations: 0; column temperature: room). 18.3 mg of the title compound was obtained.

¹H NMR (400 MHz, DMSO-d₆) δ 8.24-8.00 (m, 3H), 7.82-7.67 (m, 2H), 7.55-7.44 (m, 3H), 7.43-7.17 (m, 3H), 7.13-7.01 (m, 2H), 4.13-3.90 (m, 2H), 2.06 (s, 3H). LCMS m/z=542 [M+1]⁺.

### Bioactivity Test

### I. Enzymatic Testing Method for MAT2A

### 1. Experimental Steps

a) A 5×MAT2A test buffer (250 mM Tris-HCl, pH 8.0; 250 mM KCl; 75 mM MgCl₂; 0.025% BSA; 0.05% Brij35; 1.5 mM EDTA) was first prepared and partially diluted to 1× for later use;
b) Preparation and addition of MAT2A enzyme (BPS, 71401): the MAT2A enzyme was prepared into 3.674 ng/L by using the 1×MAT2A test buffer (1.67×, final concentration 2.20 ng/L), a BioTek (MultiFlo FX) automatic liquid separator was used, 15 μL of 1.67×MAT2A enzyme solution was added to compound test wells and negative control wells respectively, and 15 μL of 1×MAT2A test buffer was added to blank control wells;
c) Preparation and addition of a compound: a compound to be tested was diluted with DMSO from 10 mM stock solution to 100 μM, a positive drug AGI-24512 was diluted under the same conditions, and the diluted compound and positive drug were automatically sprayed into each well by using a Tecan compound titrator (D300e) according to preset concentration gradients, where the spray volume was little and negligible. The concentration gradient was initially 1 μM, the dilution was implemented by ½ log, and a total of 8 gradients were set. Centrifugation at 2500 rpm for 30 s and incubation at 25° C. for 30 min were performed;
d) Preparation of ATP: 10 mM ATP (Sigma, A7699) was diluted with 1×MAT2A test buffer to 700 μM for later use;
e) Preparation and addition of a substrate and ATP mixture: 5×MAT2A test buffer, 3 μL/well; 750 μM L-methionine (Adamas, 01100469), 2.5 μL/well; 700 μM ATP, 2.5 μL/well; and double distilled water, 2 μL/well. A total amount of required mixture was prepared according to the number of test wells, and 10 μL of the mixture was added to each well by using the BioTek (MultiFlo FX) automatic liquid separator, followed by centrifugation at 2500 rpm for 30 s and reaction at 25° C. for 150 min;
f) Addition of Biomol Green detection reagent: 50 μL of Biomol Green (Enzo, BML-AK111) was added to each well by using the BioTek (MultiFlo FX) automatic liquid separator, followed by centrifugation at 2500 rpm for 30 s and incubation at 25° C. for 20 minutes;
g) After the reaction, OD₆₂₀ values were read by using a Perkin Elmer (Envision 2105) multifunctional plate reader.

### 2. Data analysis

A formula for calculating an inhibition rate was as follows:

$$\% Inhibition = \frac{OD max - OD sample}{OD max - Od min} \times 100 \%$$

Wherein,
% Inhibition: inhibition rate %
ODsample: OD₆₂₀ value in a sample well;
ODmin: representing a mean OD₆₂₀ value of blank control wells without the enzyme or compound to be tested;
ODmax: representing a mean OD620 value of negative control wells with the enzyme and without the compound.

Then a dose-effect curve log(inhibitor) vs. response-Variable slope was fitted by usingGraphPad Prism 5 software to obtain an IC₅₀ value of compound for MAT2A enzyme inhibition.

### II. Cell Testing Method

### 1. Experimental Steps

HCT116 MTAP −/− cells (purchased from Horizon Discovery): human colorectal cancer cell lines with MTAP gene deletion were cultured in medium RPMI 1640+10% FBS (Fetal bovine serum). On day 0 of the experiment, the live cell density of the foregoing cells in logarithmic growth phase was adjusted to 5000 cells/ml, the cells were inoculated to a 96-well plate by an amount of 100 μl/well, and a blank group was set in parallel; and the inoculated cell plate was put into a 37° C., 5% CO₂ incubator for overnight culture.

On day 1 of the experiment, the overnight cultured cell plate was taken out, the supernatant was discarded, 80 μl of serum-free RPMI 1640 medium was added to each well, and place the cells in an incubator for starvation cultivation for 4 h. The compound to be tested was dissolved in DMSO (dimethyl sulfoxide) to prepare a 10 mM compound stock solution. After starvation, the cell plate was taken out, and 80 μl of RPMI 1640+20% FBS medium was added to each well; the cell plate was placed on an automatic liquid dispenser D300e (Tecan), and a dosing program was set as follows: a maximum concentration of the compound to be tested was 30 μM, the compound was diluted with DMSO by a 3 times concentration gradient, there was a total of 10 concentrations, two duplicate wells were set for each concentration, and the DMSO final concentration in each well of the 96-well plate was 0.3%, v/v. The pre-prepared 10 mM stock solution of the compound to be tested was taken out, and the above dosing program was run for dosing. After the dosing was completed, the cell plate was placed in incubator and culture for 120 h.

On day 6 of the experiment, the cell plate was taken out, 50 μl of CellTiter-Go® (purchased from Promega) was added to each well, and fluorescence signal was measured on Envision (PerkinElmer) according to operation procedures in the manual.

### 2. Data Analysis

A dose-effect curve: log(inhibitor) vs. response-Variable slope was fitted by using GraphPad Prism 5 software to obtain an IC₅₀ value of the compound for inhibiting cell proliferation inhibition. A formula for calculating an inhibition rate was:

$$Inhibition rate \% = \frac{1 - ( Signal value of tested compound - Signal value of blank group )}{Signal value of negative control group - Signal value of blank group} 100 \%$$

Wherein,
Signal value of tested compound: mean fluorescence signal of cells group+medium group+compound group;
Signal value of blank group: mean fluorescence signal of medium group;
Signal value of negative control group: mean fluorescence signal of cells group+medium group.

### 3. Experimental Results

According to the above experimental method, the IC₅₀ of AGI-24512 for inhibiting MAT2A was 23.2 nM, and the IC₅₀ value for inhibiting HCT116 MTAP−/− cells was 153.6 nM.

The following scoring levels are given based on the IC₅₀ value of the tested compounds for inhibiting MAT2A: (A) IC₅₀ value is less than 50 nM, (B) IC₅₀ value is between 50 nM and 200 nM, (C) IC₅₀ value is between 200 nM and 1000 nM, and (D) IC₅₀ value is greater than 1000 nM.

The following scoring levels are given based on the IC₅₀ value of the tested compounds for inhibiting HCT116 MTAP−/− cells: (A) IC₅₀ is less than 150 nM, (B) IC₅₀ is between 150 nM and 400 nM, (C) IC₅₀ is between 400 nM and 1000 nM, and (D) IC₅₀ is greater than 1000 nM.

The experimental results of the compounds of the present invention are shown in Table 1 below:

TABLE 1

| | MAT2A | HCT116 MTAP−/− |
|---|---|---|
| Example | inhibition | cell inhibition |
| number | score | score |
| 1 | B | — |
| 2 | A | — |
| 3 | A | A |
| 4 | A | A |
| 5 | D | — |
| 6 | A | B |
| 7 | A | C |
| 8 | D | — |
| 9 | A | A |
| 10 | A | A |
| 11 | A | A |
| 12 | A | D |
| 13 | D | — |
| 14 | A | — |
| 15 | A | C |
| 16 | A | C |
| 17 | — | — |
| 18 | A | — |
| 19 | A | — |
| 20 | A | A |
| 21 | D | — |
| 22 | A | A |
| 23 | A | A |
| 24 | A | A |
| 25 | A | A |
| 26 | A | A |
| 27 | B | D |
| 28 | A | A |
| 29 | D | — |
| 30 | D | — |
| 31 | D | — |
| 32 | A | A |
| 33 | D | — |
| 34 | D | — |
| 35 | A | — |
| 36 | — | — |
| 37 | B | — |
| 38 | A | A |
| 39 | A | A |
| 40 | D | — |
| 41 | A | A |
| 42 | A | B |

Note:
— ms untested

### III. In Vivo Effect Experiment of HCT116 MTAP^{-/-}Subcutaneous Xenograft Tumors in Nude Mice

### 1. Experimental Steps

Female Nu/Nu nude mice (6-8 weeks old, Beijing Vital River Laboratory Animal Technology Co., Ltd.) were raised in an SPF animal room at a temperature of 20° C.-25° C., relative humidity of 40%-70%, and light and dark lighting for 12 hours each; and animals can freely drink and eat. The animals were adaptively fed before experiment.

HCT116 MTAP^{-/-} cells (Horizon) were cultured in vitro for proliferation, cells in logarithmic growth phase were collected and resuspended in a serum-free RPMI-1640 medium, and the cell concentration was adjusted to 6.0×10⁷ cells/mL; and 100 μL of the cell suspension was injected subcutaneously into the right anterior armpit of each nude mouse by using a 1 mL syringe, the status of the animals was observed regularly, and the growth of transplanted tumors was monitored regularly.

When the tumor volume reached 100-300 mm³, animals with too large or too small tumor volume or uncertain tumor formation were eliminated, and tumor bearing mice with good health and similar tumor volume were selected and randomly grouped, 6 mice in each group; the administration group received daily intragastric administration (AG-270: 50 mg/kg, with a structure shown below; compound to be tested: 1-7.5 mg/kg); and the control group was intragastrically administrated with the same volume of blank solvent. In the administration period, the tumor diameter was measured twice a week, the tumor volume was calculated, the animals were weighed, and their weight was recorded.

SAM test in transplanted tumors: at the end of the experiment on day 15 of administration, the animals were euthanized by CO₂, and tumor tissues were stripped, cleaned with cold PBS, weighed, and frozen in liquid nitrogen at low temperature (−80° C.) for later use. The frozen tumor tissues were taken out and unfrozen in an ice bath, 80% methanol solution (containing 1 M formic acid) was added, a ratio of the tumor tissues to the 80% methanol solution (containing 1 M formic acid) was 1:10 (w/v), and the tissues were homogenized; the homogenate was collected and treated, and SAM (S-adenosylmethionine) was tested by LC-MS/MS.

### Ag-270 Structure:

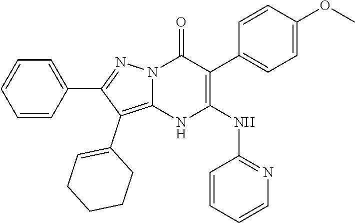 [SMILES: COc1ccc(-c2c(Nc3ccccn3)[nH]c3c(C4=CCCCC4)c(-c4ccccc4)nn3c2=O)cc1]

### 2. Data Analysis

A formula for calculating tumor volume (TV) is: Tv=½×a×b², wherein a represents long diameter of the tumor, and b represents short diameter of the tumor.

A formula for calculating relative tumor volume (RTV) is: RTV=TVₜ/TVᵢₙᵢₜᵢₐₗ, wherein TVᵢₙᵢₜᵢₐₗ is the tumor volume measured during group administration, and TVₜ is the tumor volume measured during each measurement in the administration period.

A formula for calculating relative tumor proliferation (T/C (%)) is: T/C %=(RTV_{T}/RTV_{C})×100%, wherein RTV_{T} represents relative tumor volume of the treatment group, and RTV_{C} represented relative tumor volume of the solvent control group.

A formula for calculating tumor growth inhibition (TGI) (%) is: TGI=[1−(TVₜ(T)−TVᵢₙᵢₜᵢₐₗ(T))/(TV_{t(C)}−TVᵢₙᵢₜᵢₐₗ(c))]×100%, wherein TVₜ(T) represented tumor volume measured each time in the treatment group, TVᵢₙᵢₜᵢₐₗ(T) represents tumor volume of the treatment group during group administration, TV_{t(C)} represents tumor volume measured each time in the solvent control group, and TV_{initial(C)} represents tumor volume of the solvent control group during group administration.

A formula for calculating animal weight loss is: animal weight loss=100%×(BWᵢₙᵢₜᵢₐₗ−BWₜ)/BWᵢₙᵢₜᵢₐₗ, wherein BWₜ represents animal weight measured each time in the administration period, and BWᵢₙᵢₜᵢₐₗ represents animal weight during group administration.

A formula for calculating tumor weight inhibition IR (%) is: IR=100%×(W_{C}−W_{T})/W_{C}, wherein W_{C} represents tumor weight of the control group, and W_{T} represents tumor weight of the treatment group.

Experimental data is calculated and statistically processed by using Microsoft Office Excel 2007 software. Unless otherwise specified, the data were expressed as mean±standard error (Mean SEM), and t-test is used for comparison between two groups.

### 3. Experimental Results

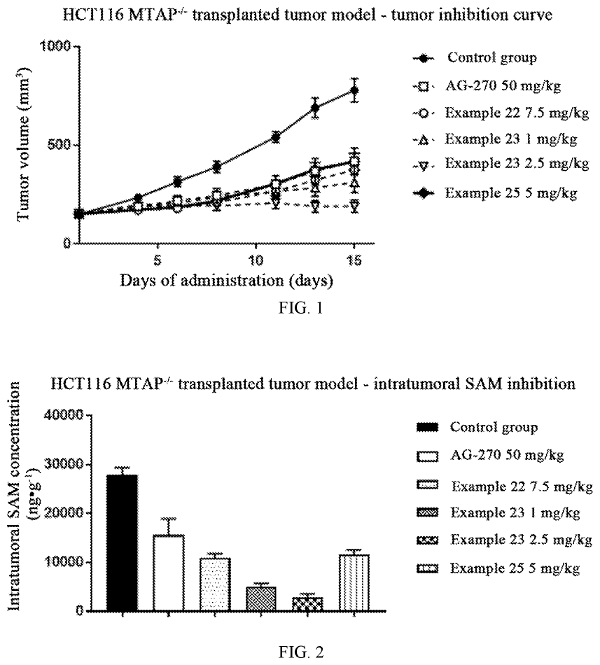

Growth curves of tumor volume in each group are shown in FIG. 1, results of SAM test in tumors of each group are shown in FIG. 2, and the mean tumor volume and SEM in each group are shown in Table 2 below:

TABLE 2
Tumor volume (mm³)

| | Control | | AG-270 | | Example 22 | | Example 23 | | Example 23 | | Example 25 | |
|---|---|---|---|---|---|---|---|---|---|---|---|---|
| Days of | group | | 50 mg/kg | | 7.5 mg/kg | | 1 mg/kg | | 2.5 mg/kg | | 5 mg/kg | |
| administration | Mean | | Mean | | Mean | | Mean | | Mean | | Mean | |
| (days) | volume | SEM | volume | SEM | volume | SEM | volume | SEM | volume | SEM | volume | SEM |
| 1 | 150 | 5 | 154 | 6 | 152 | 5 | 152 | 5 | 153 | 6 | 152 | 6 |
| 4 | 234 | 12 | 189 | 18 | 173 | 15 | 198 | 20 | 190 | 17 | 174 | 14 |
| 6 | 316 | 24 | 219 | 26 | 180 | 15 | 205 | 20 | 196 | 18 | 186 | 14 |
| 8 | 390 | 28 | 244 | 37 | 212 | 18 | 238 | 29 | 193 | 23 | 219 | 19 |
| 11 | 542 | 27 | 303 | 43 | 267 | 24 | 266 | 40 | 208 | 29 | 305 | 21 |
| 13 | 689 | 50 | 369 | 65 | 324 | 24 | 284 | 43 | 190 | 29 | 378 | 35 |
| 15 | 779 | 59 | 419 | 68 | 375 | 26 | 312 | 51 | 191 | 31 | 417 | 43 |

1. A compound of formula I or a pharmaceutically acceptable salt thereof, 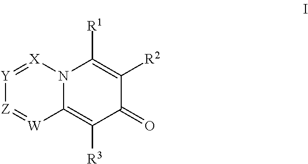
wherein X is selected from CR⁴ or N; Y is selected from CR⁵ or N; Z is selected from CR⁶ or N;
W is selected from CR⁷ or N;
wherein R¹, R⁴, R⁵, R⁶, and R⁷ are independently selected from hydrogen, cyano, C2-C6 alkynyl, C8-C10 cycloalkynyl, halogen, hydroxyl, NH₂, (C1-C6 alkyl)-NR⁸—, (C1-C6 alkyl)-O—, (C1-C6 alkyl)-S—, C1-C6 alkyl, C3-C6 cycloalkyl, 6-10-membered aryl, C2-C6 alkenyl, or C3-C6 cycloalkenyl; the C1-C6 alkyl, (C1-C6 alkyl)-NR⁸—, (C1-C6 alkyl)-O—, C2-C6 alkenyl, C3-C6 cycloalkyl, or C3-C6 cycloalkenyl is optionally substituted by halogen, cyano, hydroxyl, —NR⁸R⁹, C1-C3 alkyl, C1-C3 alkoxy, C2-C6 alkenyl, or C2-C6 alkynyl; the 6-10-membered aryl is optionally substituted by halogen, hydroxyl, cyano, —NR⁸R⁹, NO₂, C1-C3 alkyl, C1-C3 alkoxy, C2-C6 alkenyl, or C2-C6 alkynyl; or the 6-10-membered aryl is optionally substituted by halogen, hydroxyl, cyano, —NR⁸R⁹, or NO₂ substituted C1-C3 alkyl, C1-C3 alkoxy, C2-C6 alkenyl, or C2-C6 alkynyl;
R² and R³ are independently selected from 6-10-membered aryl or 9-18-membered benzoheterocyclyl; the 6-10-membered aryl or 9-18-membered benzoheterocyclyl is optionally substituted by halogen, hydroxyl, cyano, —NR⁸R⁹, NO₂, —NR¹⁰C(O)R¹¹, C1-C6 alkyl, (C1-C6 alkyl)-O—, —C(O)NR¹⁰R¹¹, or 5-7-membered heteroaryl; and the C1-C6 alkyl, (C1-C6 alkyl)-O—, or 5-7-membered heteroaryl is optionally substituted by halogen, cyano, hydroxyl, C1-C3 alkyl, (C1-C3 alkyl)-O—, or —NR⁸R⁹;
R⁸, R⁹, R¹⁰, and R¹¹ are independently selected from H or C1-C6 alkyl; and
provided that at most 2 of W, X, Y, and Z are simultaneously N.

2. The compound or a pharmaceutically acceptable salt thereof according to claim 1, wherein the compound has a structure of formula II or formula III below: 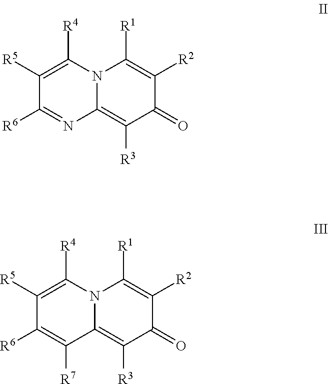
wherein R¹, R², R³, R⁴, R⁵, R⁶, R⁷, R⁸, R⁹, R¹⁰, and R¹¹ are defined as claim 1.

3. The compound or a pharmaceutically acceptable salt thereof according to claim 1, wherein R¹, R⁴, R⁵, R⁶, and R⁷ are independently selected from hydrogen, halogen, hydroxyl, NH₂, (C1-C6 alkyl)-NR⁸—, (C1-C6 alkyl)-O—, C1-C6 alkyl, C3-C6 cycloalkyl, or 6-10-membered aryl, wherein the C1-C6 alkyl, (C1-C6 alkyl)-NR⁸—, (C1-C6 alkyl)-O—, or C3-C6 cycloalkyl is optionally substituted by halogen, cyano, hydroxyl, or —NR⁸R⁹; the 6-10-membered aryl is optionally substituted by halogen substituted C1-C3 alkoxy;
preferably, R¹, R⁴, R⁵, R⁶, and R⁷ are independently selected from hydrogen, halogen, hydroxyl, NH₂, (C1-C6 alkyl)-NR⁸—, (C1-C6 alkyl)-O—, C1-C6 alkyl, C3-C6 cycloalkyl, or 6-10-membered aryl, wherein the C1-C6 alkyl, (C1-C6 alkyl)-NR⁸—, (C1-C6 alkyl)-O—, or C3-C6 cycloalkyl is optionally substituted by halogen; the 6-10-membered aryl is optionally substituted by halogen substituted C1-C3 alkoxy;
more preferably, R¹, R⁴, R⁵, R⁶, and R⁷ are independently selected from hydrogen, halogen, hydroxyl, NH₂, (C1-C6 alkyl)-NR⁸—, (C1-C6 alkyl)-O—, C1-C6 alkyl, C3-C6 cycloalkyl, or 6-10-membered aryl, wherein the C1-C6 alkyl, (C1-C6 alkyl)-NR⁸—, (C1-C6 alkyl)-O—, or C3-C6 cycloalkyl is optionally substituted by fluorine; the 6-10-membered aryl is optionally substituted by fluorine substituted C1-C3 alkoxy;
preferably, R¹ is hydrogen; and
preferably, R⁷ is hydrogen.

4. The compound or a pharmaceutically acceptable salt thereof according to claim 1, wherein R⁴ is selected from hydrogen, C1-C6 alkyl, or C3-C6 cycloalkyl;
preferably, R⁴ is selected from hydrogen, cyclopropyl, or C1-C6 alkyl; preferably, R⁴ is selected from hydrogen or C1-C6 alkyl; more preferably, R⁴ is selected from hydrogen, methyl, or cyclopropyl; further preferably, R⁴ is selected from hydrogen or methyl;
preferably, R⁵ is selected from hydrogen or C1-C6 alkyl; preferably, R⁵ is selected from hydrogen or methyl; more preferably, R⁵ is selected from hydrogen; and
preferably, R⁶ is selected from hydrogen, chlorine, hydroxyl, cyclopropyl, CF₃CH₂O—, CHF₂O—, CF₃CH₂NH—, 4-difluoromethoxylphenyl, or CH₃CH₂O—; preferably, R⁶ is selected from cyclopropyl, CF₃CH₂O—, CF₃CH₂NH—, or CH₃CH₂O—; and more preferably, R⁶ is selected from cyclopropyl, CF₃CH₂O—, or CF₃CH₂NH—.

5. The compound or a pharmaceutically acceptable salt thereof according to claim 1, wherein R² and R³ are independently selected from phenyl, naphthyl, 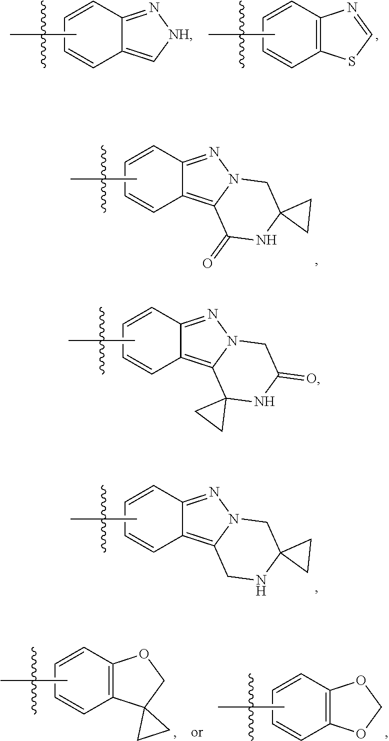
wherein the group is optionally substituted by halogen, hydroxyl, cyano, —NR⁸R⁹, NO₂, —NR¹⁰C(O)R¹¹, C1-C6 alkyl, (C1-C6 alkyl)-O—, —C(O)NR¹⁰R¹¹, or 5-7-membered heteroaryl; the C1-C6 alkyl, (C1-C6 alkyl)-O—, or 5-7-membered heteroaryl is optionally substituted by halogen, cyano, hydroxyl, C1-C3 alkyl, (C1-C3 alkyl)-O—, or —NR⁸R⁹;
preferably, R² and R³ are independently selected from phenyl, naphthyl, 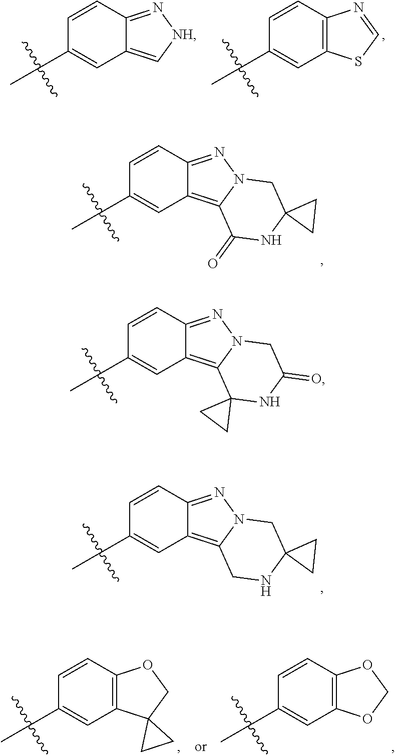
wherein the group is optionally substituted by halogen, hydroxyl, cyano, —NR⁸R⁹, NO₂, —NR¹⁰C(O)R¹¹, C1-C6 alkyl, (C1-C6 alkyl)-O—, —C(O)NR¹⁰R¹¹, or 5-7-membered heteroaryl; the C1-C6 alkyl, (C1-C6 alkyl)-O—, or 5-7-membered heteroaryl is optionally substituted by halogen, cyano, hydroxyl, C1-C3 alkyl, (C1-C3 alkyl)-O—, or —NR⁸R⁹;
more preferably, R² and R³ are independently selected from phenyl, naphthyl, 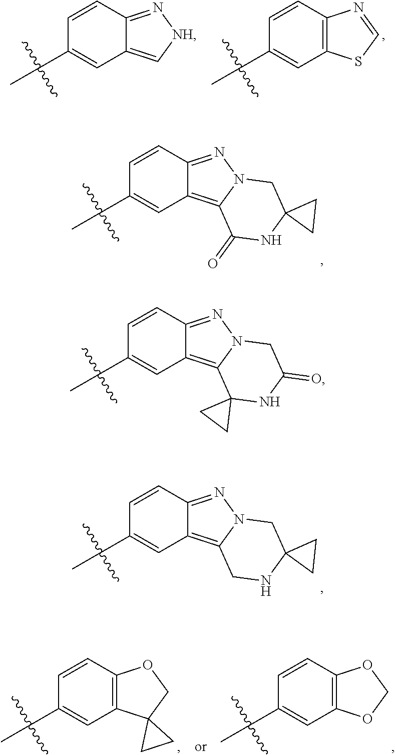
wherein the group is optionally substituted by halogen, cyano, —NR⁸R⁹, —NR¹⁰C(O)R¹¹, C1-C6 alkyl, (C1-C6 alkyl)-O—, or 5-7-membered heteroaryl; and the C1-C6 alkyl, (C1-C6 alkyl)-O—, or 5-7-membered heteroaryl is optionally substituted by methyl, halogen, or cyano;
wherein R⁸, R⁹, R¹⁰, and R¹¹ are independently selected from H or C1-C6 alkyl;
preferably, R⁸, R⁹, and R¹⁰ are independently H; and
preferably, R¹¹ is selected from C1-C6 alkyl; and preferably, R¹¹ is methyl.

6. The compound or a pharmaceutically acceptable salt thereof according to claim 1, wherein R² is selected from phenyl, 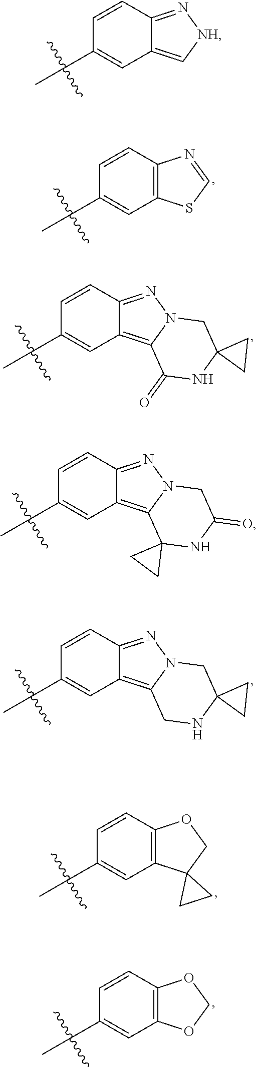
wherein the group is optionally substituted by difluoromethoxy, methyl, NH₂, methoxy, fluorine, cyanomethyl, CH₃CONH—, 1-methyl-1H-imidazol-4-yl, or 1-methyl-1H-pyrazol-5-yl; preferably, the group is optionally substituted by difluoromethoxy, methyl, NH₂, methoxy, fluorine, cyanomethyl, or 1-methyl-1H-pyrazol-5-yl;
preferably, R² is selected from 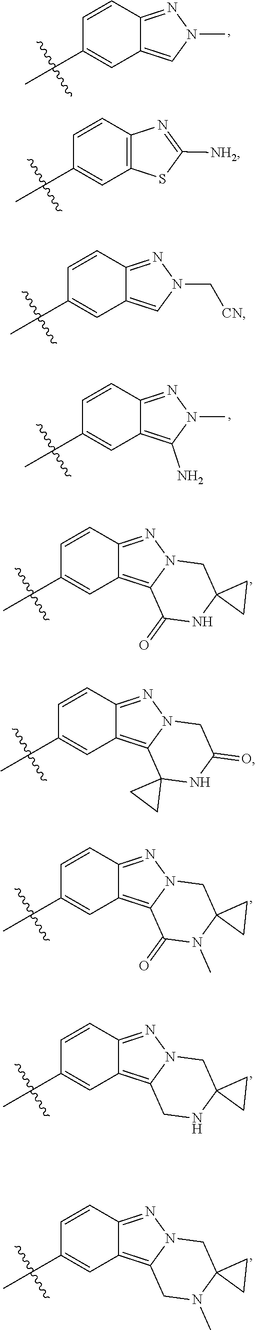 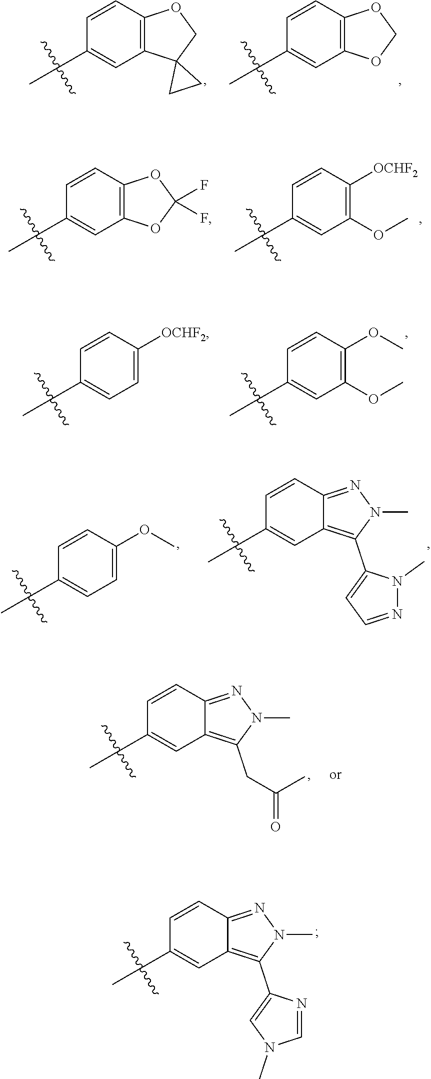
preferably, R² is selected from 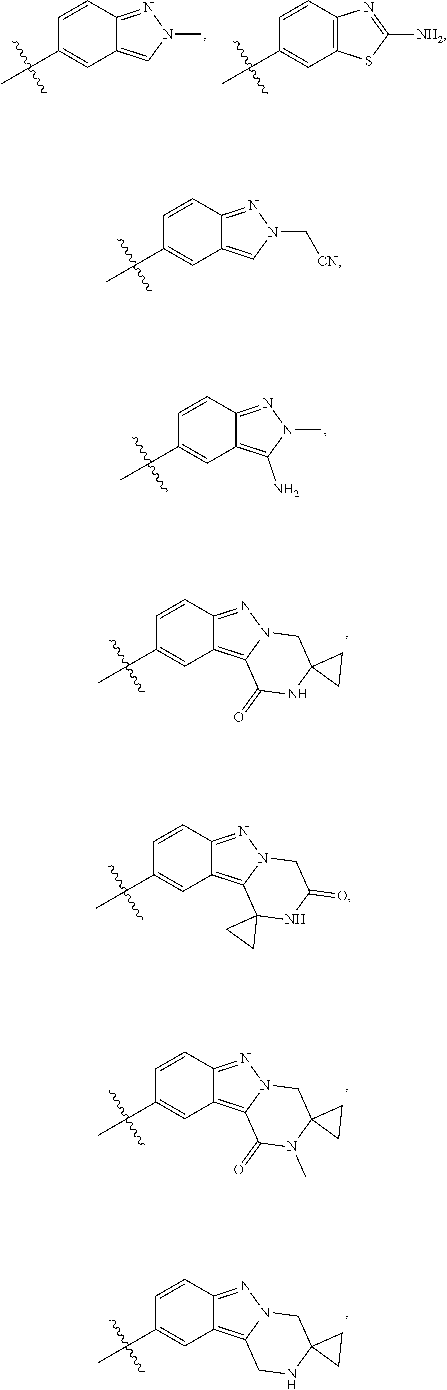 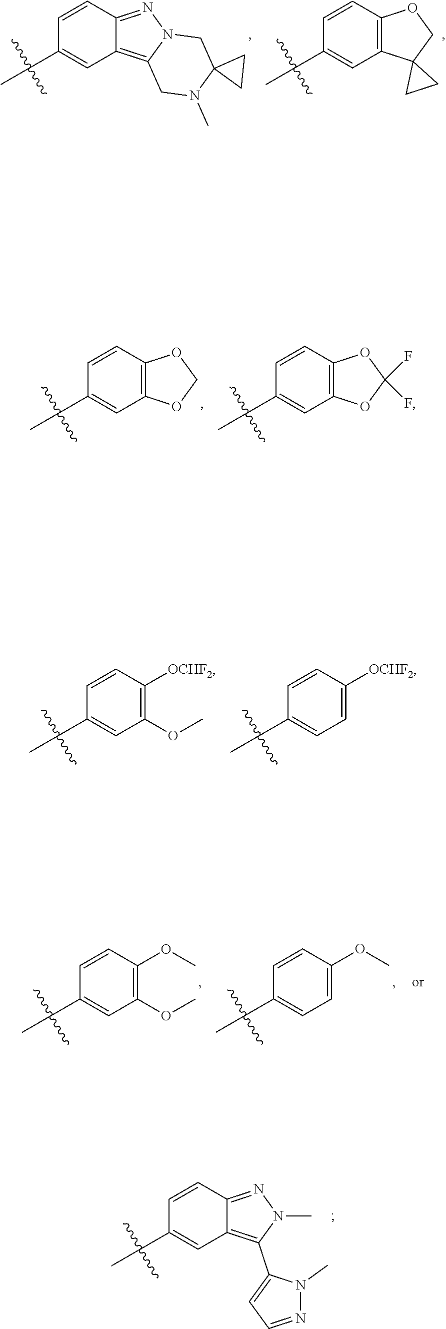
more preferably, R² is selected from 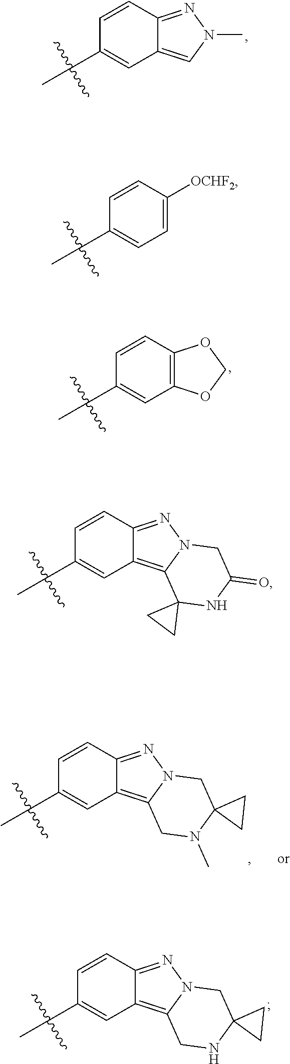 [SMILES: *c1ccc2nn(C)cc2c1 ; *c1ccc(OC(F)F)cc1 ; *c1ccc2c(c1)OCO2 ; *c1ccc2nn3c(c2c1)C1(CC1)NC(=O)C3 ; *c1ccc2nn3c(c2c1)CN(C)C1(CC1)C3 ; *c1ccc2nn3c(c2c1)CNC1(CC1)C3]
further preferably, R² is selected from 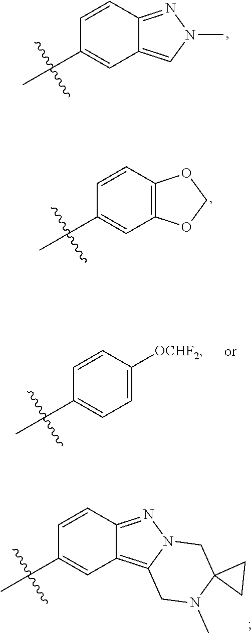 [SMILES: *c1ccc2nn(C)cc2c1 ; *c1ccc2c(c1)OCO2 ; *c1ccc(OC(F)F)cc1 ; *c1ccc2nn3c(c2c1)CN(C)C1(CC1)C3]
preferably, R³ is selected from phenyl, 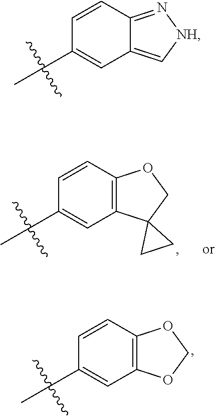 [SMILES: *c1ccc2n[nH]cc2c1 ; *c1ccc2c(c1)C1(CC1)CO2 ; *c1ccc2c(c1)OCO2]
wherein the group is optionally substituted by difluoromethoxy, methyl, NH₂, fluorine, methoxy, CH₃CONH—, 1-methyl-1H-imidazol-4-yl, or 1-methyl-1H-pyrazol-5-yl; preferably, the group is optionally substituted by difluoromethoxy, methyl, NH₂, fluorine, methoxy, or 1-methyl-1H-pyrazol-5-yl;
preferably, R³ is selected from 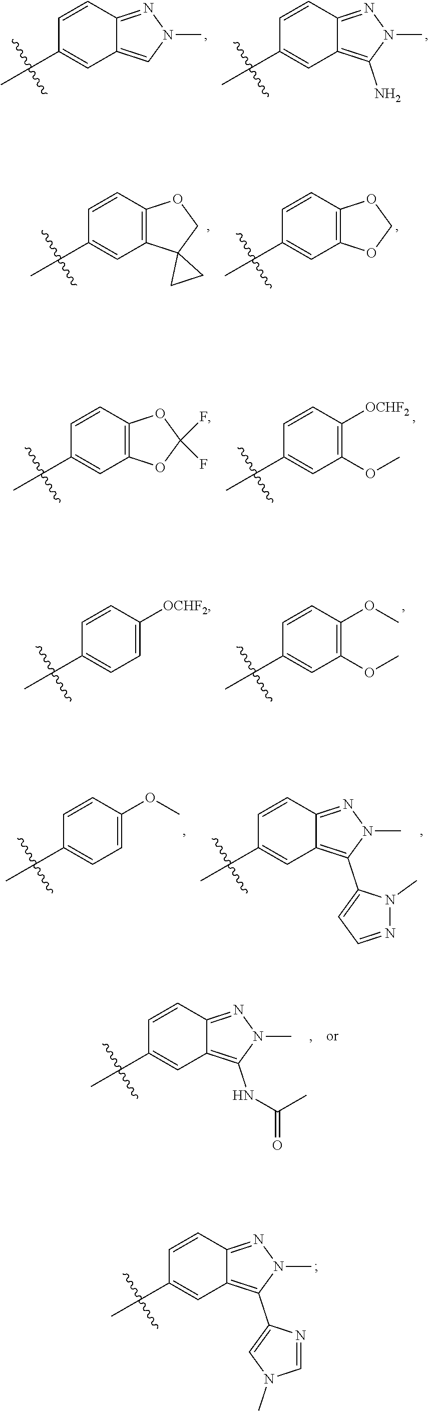
preferably, R³ is selected from 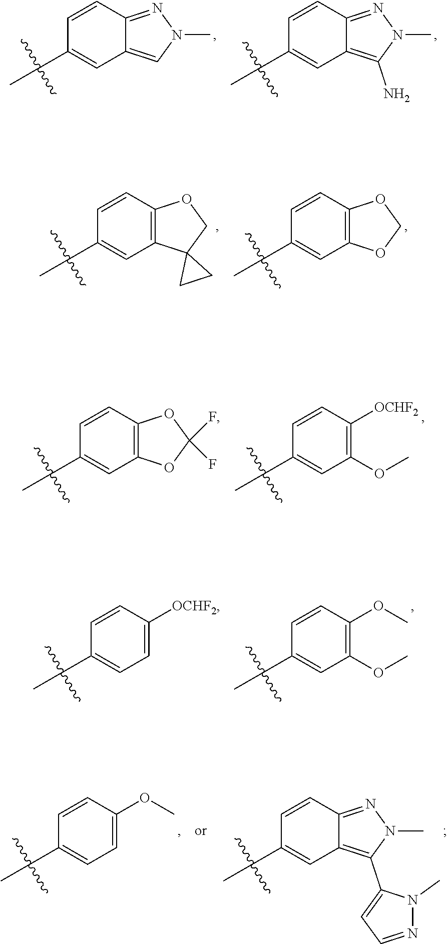
more preferably R³ is selected from 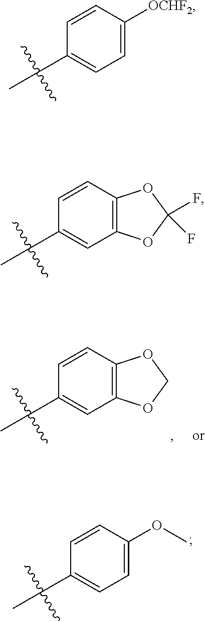
and
further preferably R³ is selected from

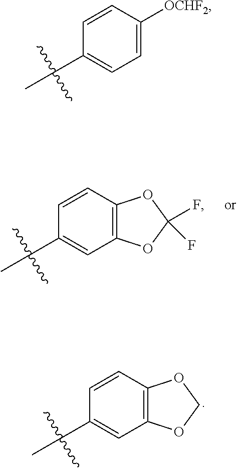 [SMILES: *c1ccc(OC(F)F)cc1 ; *c1ccc2c(c1)OC(F)(F)O2 ; *c1ccc2c(c1)OCO2]

7. A compound selected from the following group, or a pharmaceutically acceptable salt thereof,

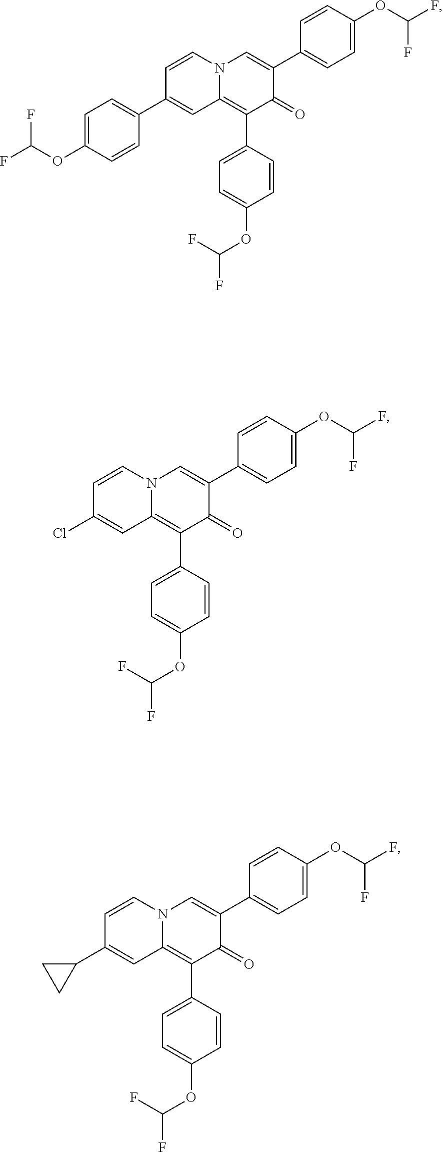 [SMILES: O=c1c(-c2ccc(OC(F)F)cc2)cn2ccc(-c3ccc(OC(F)F)cc3)cc2c1-c1ccc(OC(F)F)cc1 ; O=c1c(-c2ccc(OC(F)F)cc2)cn2ccc(Cl)cc2c1-c1ccc(OC(F)F)cc1 ; O=c1c(-c2ccc(OC(F)F)cc2)cn2ccc(C3CC3)cc2c1-c1ccc(OC(F)F)cc1]

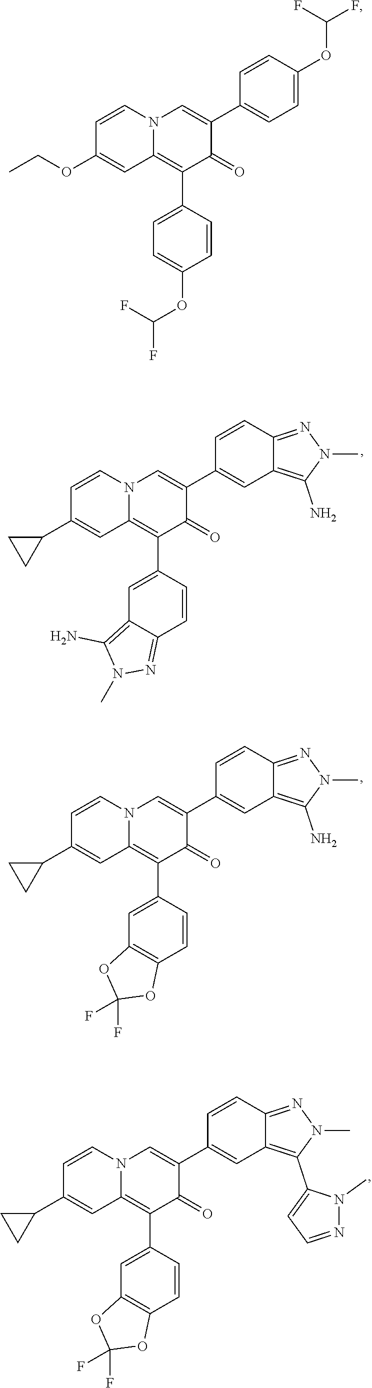 [SMILES: CCOc1ccn2cc(-c3ccc(OC(F)F)cc3)c(=O)c(-c3ccc(OC(F)F)cc3)c2c1 ; Cn1nc2ccc(-c3cn4ccc(C5CC5)cc4c(-c4ccc5nn(C)c(N)c5c4)c3=O)cc2c1N ; Cn1nc2ccc(-c3cn4ccc(C5CC5)cc4c(-c4ccc5c(c4)OC(F)(F)O5)c3=O)cc2c1N ; Cn1nccc1-c1c2cc(-c3cn4ccc(C5CC5)cc4c(-c4ccc5c(c4)OC(F)(F)O5)c3=O)ccc2nn1C]

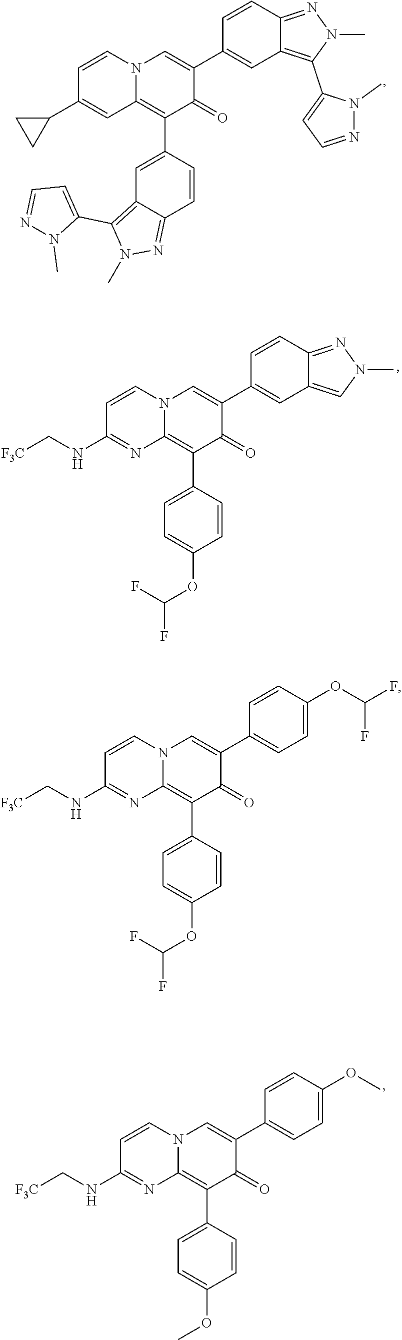 [SMILES: Cn1nccc1-c1c2cc(-c3cn4ccc(C5CC5)cc4c(-c4ccc5nn(C)c(-c6ccnn6C)c5c4)c3=O)ccc2nn1C ; Cn1cc2cc(-c3cn4ccc(NCC(F)(F)F)nc4c(-c4ccc(OC(F)F)cc4)c3=O)ccc2n1 ; O=c1c(-c2ccc(OC(F)F)cc2)cn2ccc(NCC(F)(F)F)nc2c1-c1ccc(OC(F)F)cc1 ; COc1ccc(-c2cn3ccc(NCC(F)(F)F)nc3c(-c3ccc(OC)cc3)c2=O)cc1]

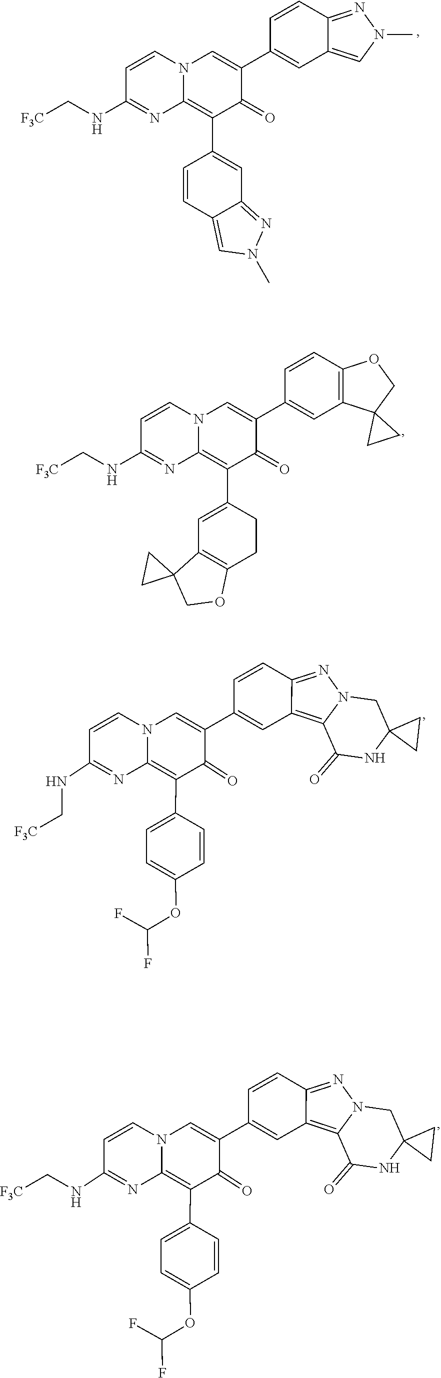 [SMILES: Cn1cc2cc(-c3cn4ccc(NCC(F)(F)F)nc4c(-c4ccc5cn(C)nc5c4)c3=O)ccc2n1 ; O=c1c(-c2ccc3c(c2)C2(CC2)CO3)cn2ccc(NCC(F)(F)F)nc2c1C1=CC2=C(CC1)OCC21CC1 ; O=C1NC2(CC2)Cn2nc3ccc(-c4cn5ccc(NCC(F)(F)F)nc5c(-c5ccc(OC(F)F)cc5)c4=O)cc3c21 ; O=C1NC2(CC2)Cn2nc3ccc(-c4cn5ccc(NCC(F)(F)F)nc5c(-c5ccc(OC(F)F)cc5)c4=O)cc3c21]

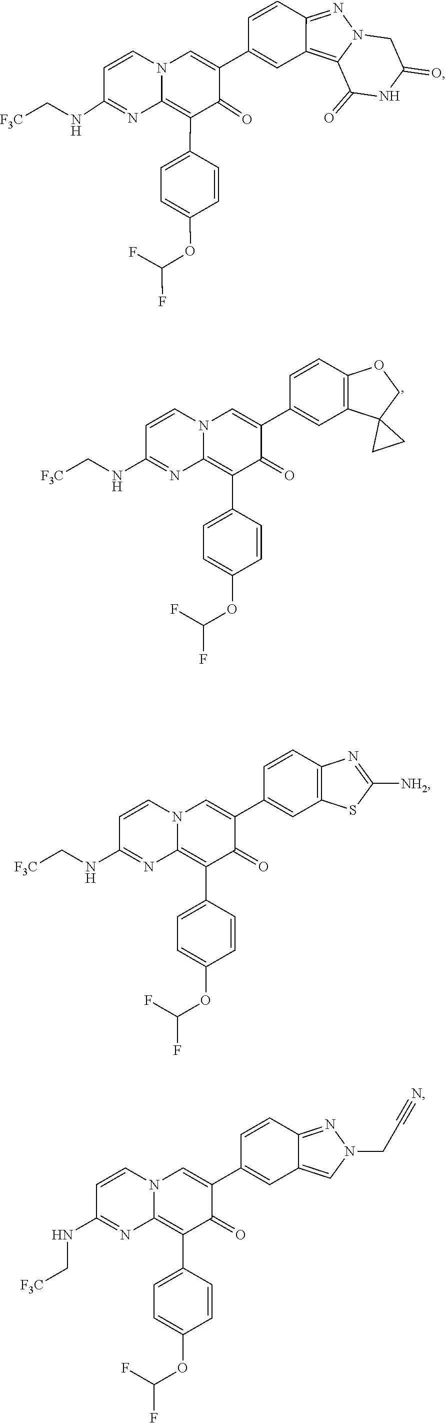 [SMILES: O=C1Cn2nc3ccc(-c4cn5ccc(NCC(F)(F)F)nc5c(-c5ccc(OC(F)F)cc5)c4=O)cc3c2C(=O)N1 ; O=c1c(-c2ccc3c(c2)C2(CC2)CO3)cn2ccc(NCC(F)(F)F)nc2c1-c1ccc(OC(F)F)cc1 ; Nc1nc2ccc(-c3cn4ccc(NCC(F)(F)F)nc4c(-c4ccc(OC(F)F)cc4)c3=O)cc2s1 ; N#CCn1cc2cc(-c3cn4ccc(NCC(F)(F)F)nc4c(-c4ccc(OC(F)F)cc4)c3=O)ccc2n1]

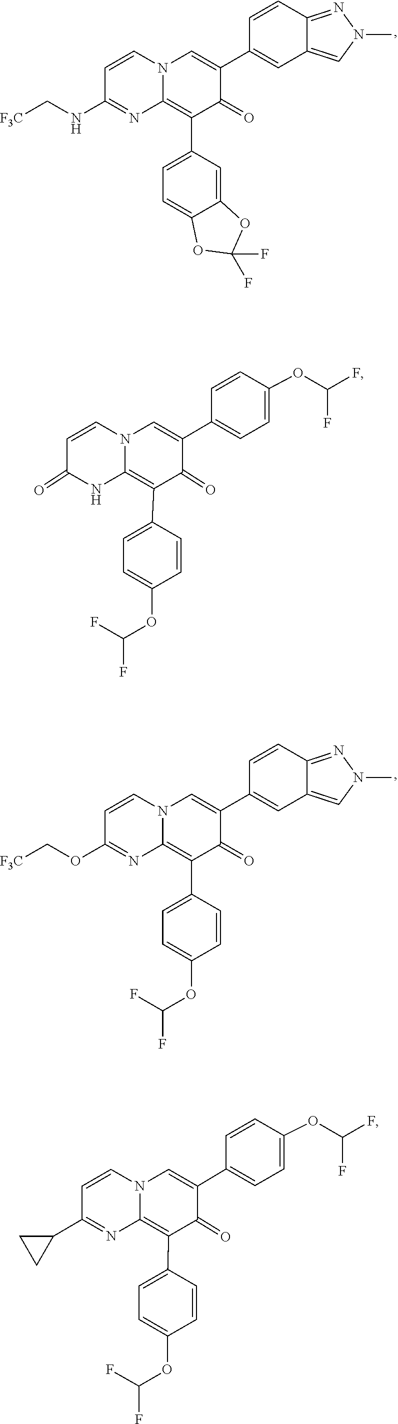 [SMILES: Cn1cc2cc(-c3cn4ccc(NCC(F)(F)F)nc4c(-c4ccc5c(c4)OC(F)(F)O5)c3=O)ccc2n1 ; O=c1ccn2cc(-c3ccc(OC(F)F)cc3)c(=O)c(-c3ccc(OC(F)F)cc3)c2[nH]1 ; Cn1cc2cc(-c3cn4ccc(OCC(F)(F)F)nc4c(-c4ccc(OC(F)F)cc4)c3=O)ccc2n1 ; O=c1c(-c2ccc(OC(F)F)cc2)cn2ccc(C3CC3)nc2c1-c1ccc(OC(F)F)cc1]

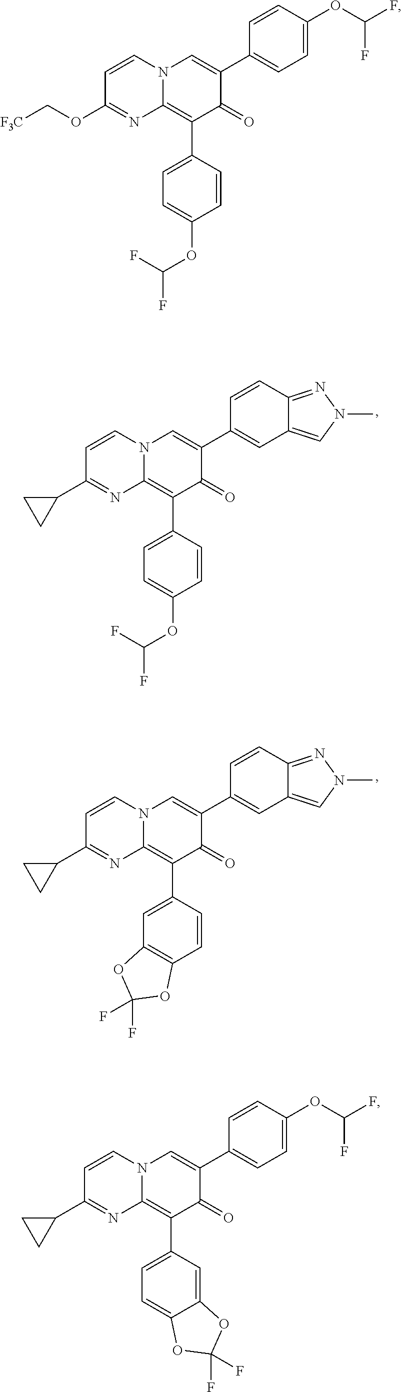 [SMILES: O=c1c(-c2ccc(OC(F)F)cc2)cn2ccc(OCC(F)(F)F)nc2c1-c1ccc(OC(F)F)cc1 ; Cn1cc2cc(-c3cn4ccc(C5CC5)nc4c(-c4ccc(OC(F)F)cc4)c3=O)ccc2n1 ; Cn1cc2cc(-c3cn4ccc(C5CC5)nc4c(-c4ccc5c(c4)OC(F)(F)O5)c3=O)ccc2n1 ; O=c1c(-c2ccc(OC(F)F)cc2)cn2ccc(C3CC3)nc2c1-c1ccc2c(c1)OC(F)(F)O2]

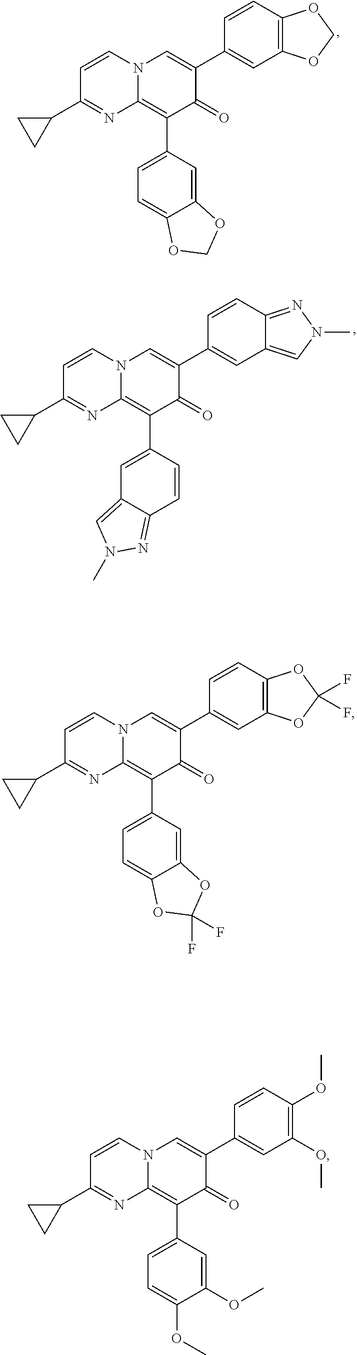 [SMILES: O=c1c(-c2ccc3c(c2)OCO3)cn2ccc(C3CC3)nc2c1-c1ccc2c(c1)OCO2 ; Cn1cc2cc(-c3cn4ccc(C5CC5)nc4c(-c4ccc5nn(C)cc5c4)c3=O)ccc2n1 ; O=c1c(-c2ccc3c(c2)OC(F)(F)O3)cn2ccc(C3CC3)nc2c1-c1ccc2c(c1)OC(F)(F)O2 ; COc1ccc(-c2cn3ccc(C4CC4)nc3c(-c3ccc(OC)c(OC)c3)c2=O)cc1OC]

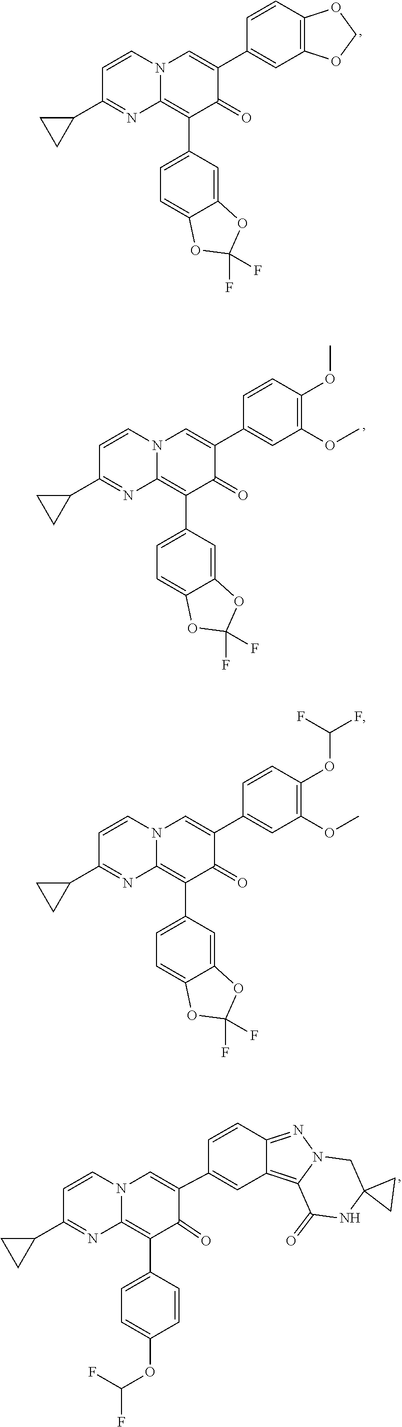 [SMILES: O=c1c(-c2ccc3c(c2)OCO3)cn2ccc(C3CC3)nc2c1-c1ccc2c(c1)OC(F)(F)O2 ; COc1ccc(-c2cn3ccc(C4CC4)nc3c(-c3ccc4c(c3)OC(F)(F)O4)c2=O)cc1OC ; COc1cc(-c2cn3ccc(C4CC4)nc3c(-c3ccc4c(c3)OC(F)(F)O4)c2=O)ccc1OC(F)F ; O=C1NC2(CC2)Cn2nc3ccc(-c4cn5ccc(C6CC6)nc5c(-c5ccc(OC(F)F)cc5)c4=O)cc3c21]

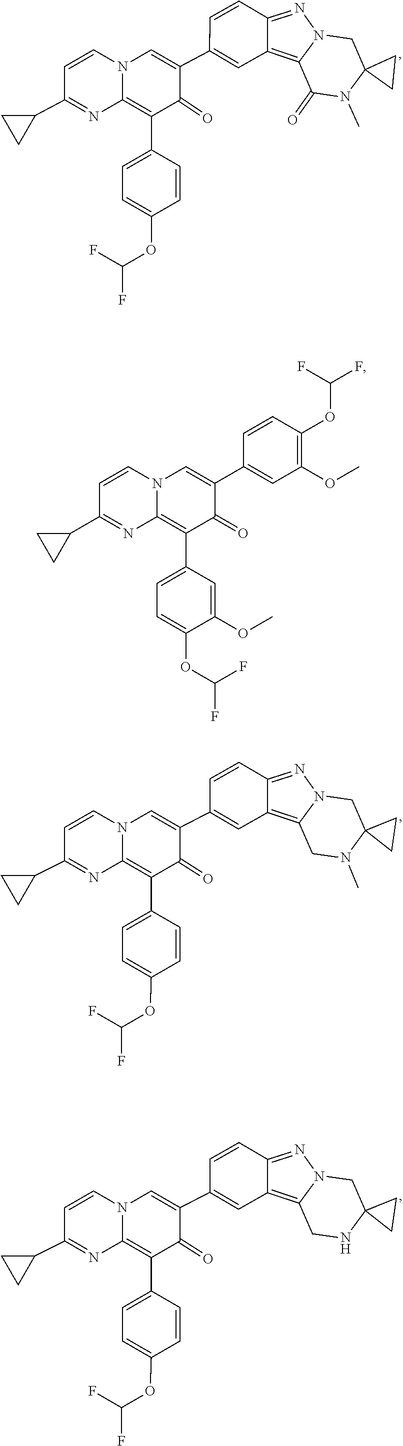 [SMILES: CN1C(=O)c2c3cc(-c4cn5ccc(C6CC6)nc5c(-c5ccc(OC(F)F)cc5)c4=O)ccc3nn2CC12CC2 ; COc1cc(-c2cn3ccc(C4CC4)nc3c(-c3ccc(OC(F)F)c(OC)c3)c2=O)ccc1OC(F)F ; CN1Cc2c3cc(-c4cn5ccc(C6CC6)nc5c(-c5ccc(OC(F)F)cc5)c4=O)ccc3nn2CC12CC2 ; O=c1c(-c2ccc3nn4c(c3c2)CNC2(CC2)C4)cn2ccc(C3CC3)nc2c1-c1ccc(OC(F)F)cc1]

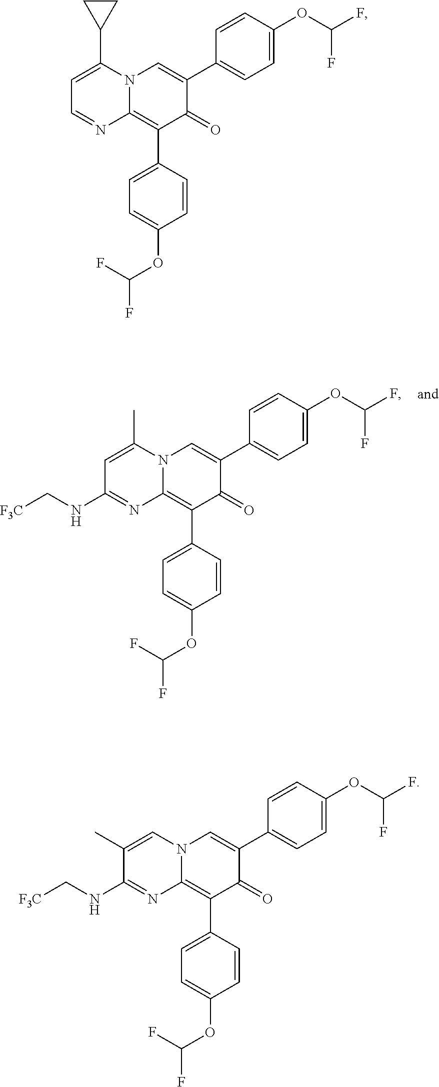 [SMILES: O=c1c(-c2ccc(OC(F)F)cc2)cn2c(C3CC3)ccnc2c1-c1ccc(OC(F)F)cc1 ; Cc1cc(NCC(F)(F)F)nc2c(-c3ccc(OC(F)F)cc3)c(=O)c(-c3ccc(OC(F)F)cc3)cn12 ; Cc1cn2cc(-c3ccc(OC(F)F)cc3)c(=O)c(-c3ccc(OC(F)F)cc3)c2nc1NCC(F)(F)F]

8. A pharmaceutical composition, comprising the compound or a pharmaceutically acceptable salt thereof according to claim 1 and a pharmaceutically acceptable carrier.

9. A method of preventing and/or treating a disease or condition mediated by overexpression of MAT2A in a subject in need, comprising administering the compound or a pharmaceutically acceptable salt thereof according to claim 1 or a pharmaceutical composition, the pharmaceutical composition comprising the compound or a pharmaceutically acceptable salt thereof.

10. A preparation method for the compound or a pharmaceutically acceptable salt thereof according to claim 1, wherein the method is selected from the following synthesis schemes: 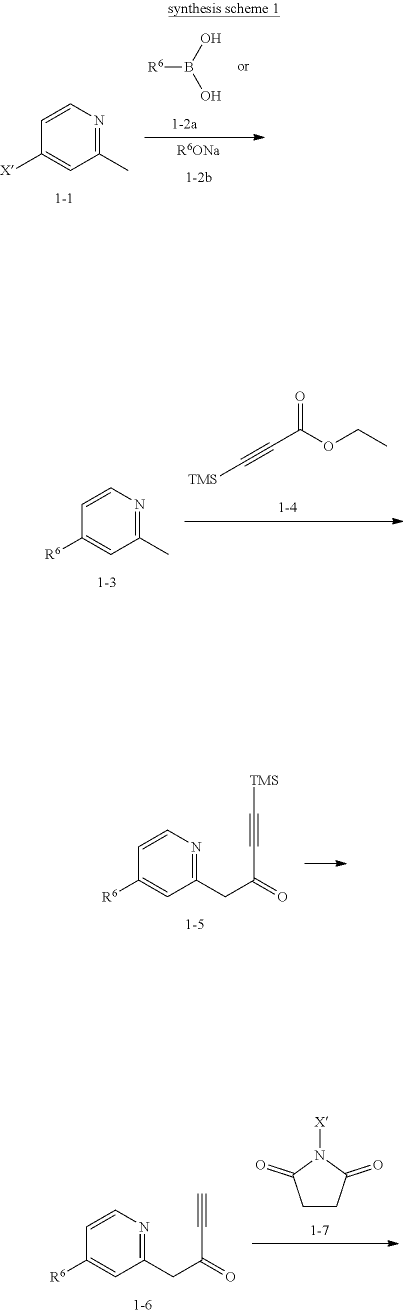 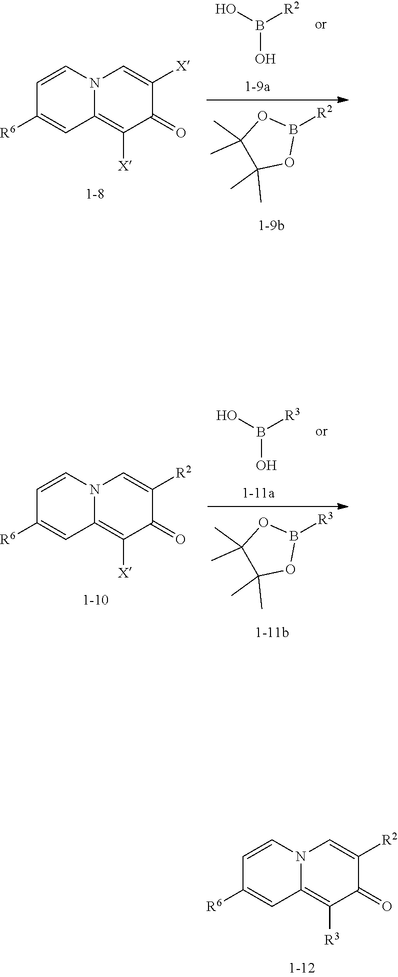
wherein X′ is selected from chlorine, bromine, or iodine; R⁶, R², and R³ are defined as in the compound of formula I;
a compound of formula 1-1 is condensed with a compound of formula 1-2a or 1-2b under alkaline conditions to obtain a compound of formula 1-3, the compound of formula 1-3 reacts with a compound of formula 1-4 to obtain a compound of formula 1-5, trimethylsilyl is removed from the compound of formula 1-5 to obtain a compound of formula 1-6, the compound of formula 1-6 is halogenated under the action of a compound of formula 1-7 as a halogenation reagent to obtain a compound of formula 1-8, the compound of formula 1-8 is condensed with a compound of formula 1-9a or 1-9b under alkaline conditions to obtain a compound of formula 1-10, and the compound of formula 1-10 is condensed with a compound of formula 1-11a or 1-11b under alkaline conditions to obtain a compound of formula 1-12;
the compound of formula 1-4 is ethyl 3-(trimethylsilyl) propiolate; 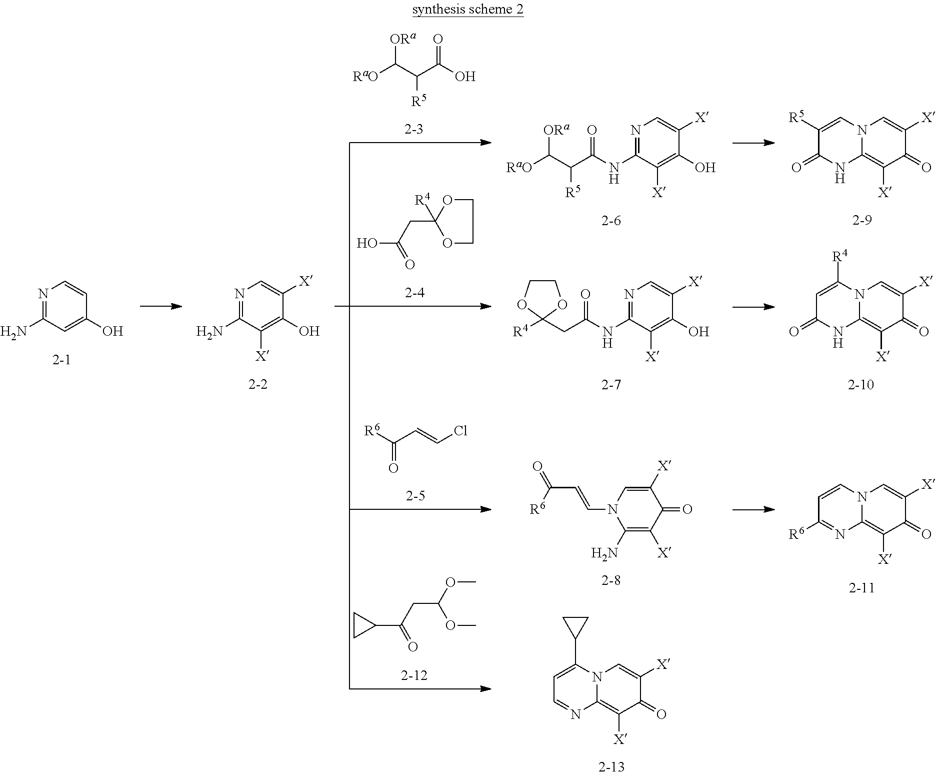
wherein X′ is selected from chlorine, bromine, or iodine; R^{a} is selected from C1-C3 alkyl; R⁴, R⁵, and R⁶ are defined as in the compound of formula I;
a compound of formula 2-1 is halogenated under the action of a halogenation reagent to obtain a compound of formula 2-2, the compound of formula 2-2 is condensed with a compound of formula 2-3 to obtain a compound of formula 2-6, and a compound of formula 2-6 is cyclized under acidic conditions to obtain a compound of formula 2-9; or
a compound of formula 2-2 is condensed with a compound of formula 2-4 to obtain a compound of formula 2-7, the compound of formula 2-7 is cyclized under acidic conditions, and then hydroxyl is removed under acidic conditions to obtain a compound of formula 2-10; or
a compound of formula 2-2 is condensed with a compound of formula 2-5 under alkaline conditions to obtain a compound of formula 2-8, and the compound of formula 2-8 is cyclized under acidic conditions to obtain a compound of formula 2-11; or
a compound of formula 2-2 is cyclized with a compound of formula 2-12 to obtain a compound of formula 2-13; 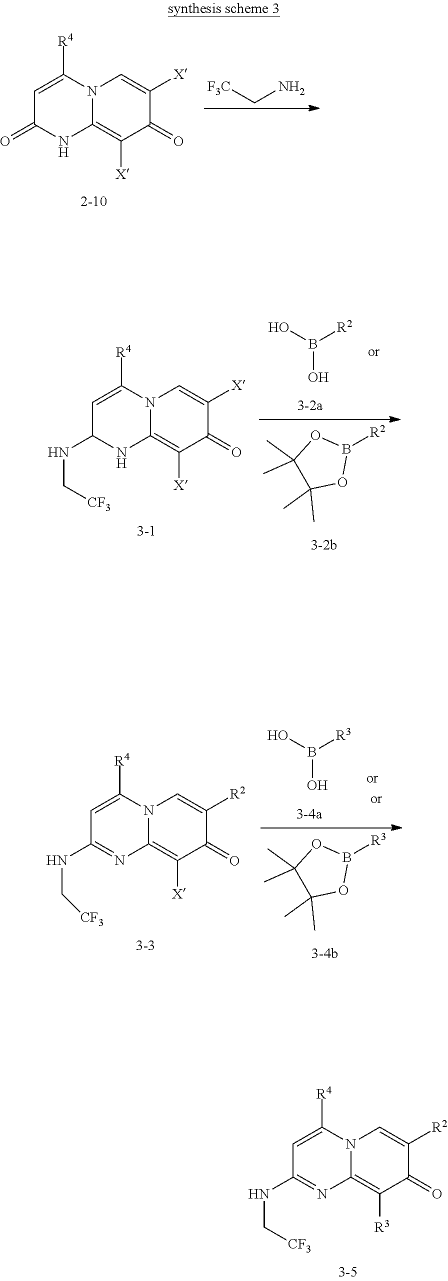
wherein X′ is selected from chlorine, bromine, or iodine; R⁴, R², and R³ are defined as in the compound of formula I;
a compound of formula 2-10 reacts with trifluoroethylamine to obtain a compound of formula 3-1, the compound of formula 3-1 is condensed with a compound of formula 3-2a or 3-2b under alkaline conditions to obtain a compound of formula 3-3, and the compound of formula 3-3 is condensed with a compound of formula 3-4a or 3-4b under alkaline conditions to obtain a compound of formula 3-5;
synthesis scheme 4: 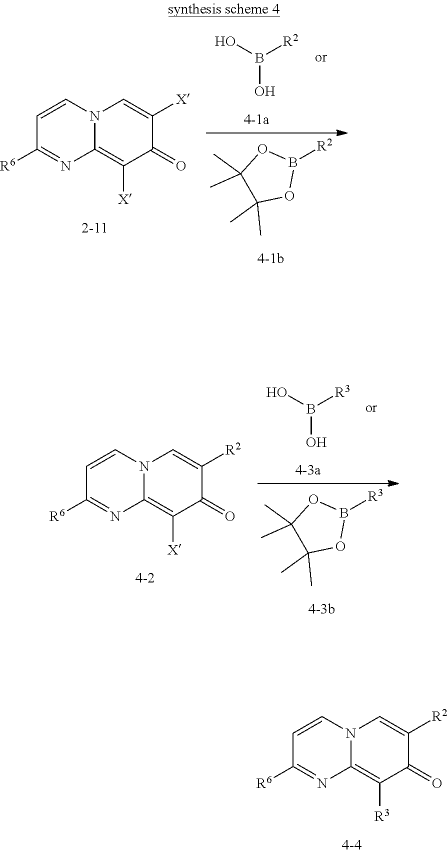
wherein X′ is selected from chlorine, bromine, or iodine; R⁶, R², and R³ are defined as in the compound of formula I;
a compound of formula 2-11 is condensed with a compound of formula 4-1a or 4-1b under alkaline conditions to obtain a compound of formula 4-2, and the compound of formula 4-2 is condensed with a compound of formula 4-3a or 3-3b under alkaline conditions to obtain a compound of formula 4-4; 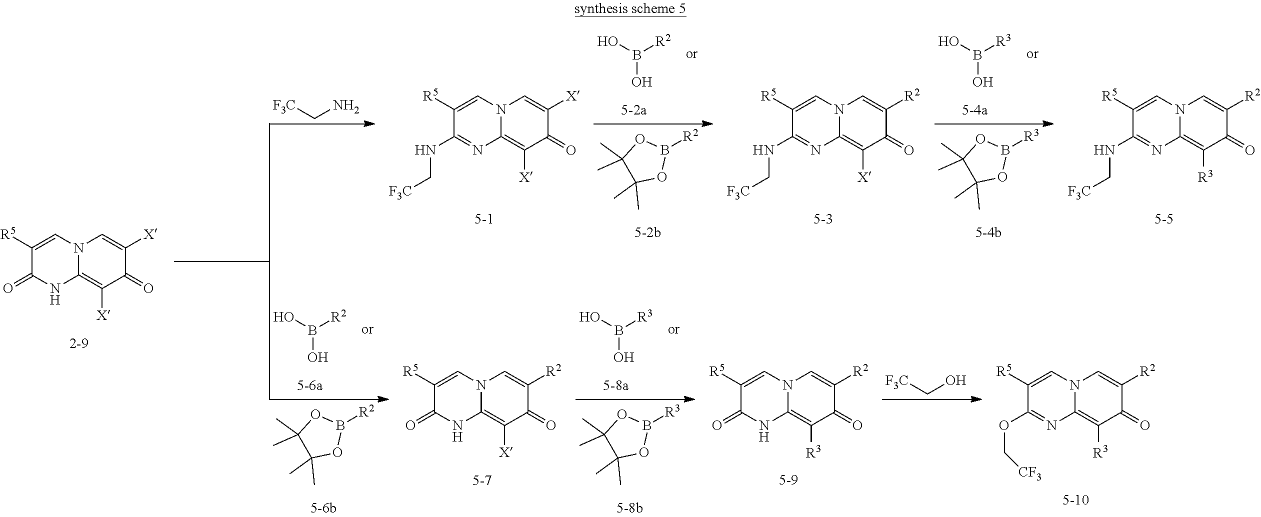
wherein X′ is selected from chlorine, bromine, or iodine; R⁵, R², and R³ are defined as in the compound of formula I;
a compound of formula 2-9 reacts with trifluoroethylamine under the action of a condensing agent to obtain a compound of formula 5-1, the compound of formula 5-1 is condensed with a compound of formula 5-2a or 5-2b under alkaline conditions to obtain a compound of formula 5-3, and the compound of formula 5-3 is condensed with a compound of formula 5-4a or 5-4b under alkaline conditions to obtain a compound of formula 5-5; or
a compound of formula 2-9 is condensed with a compound of formula 5-6a or 5-6b under alkaline conditions to obtain a compound of formula 5-7, the compound of formula 5-7 is condensed with a compound of formula 5-8a or 5-8b under alkaline conditions to obtain a compound of formula 5-9, and the compound of formula 5-9 reacts with trifluoroethanol under the action of a condensing agent to obtain a compound of formula 5-10; 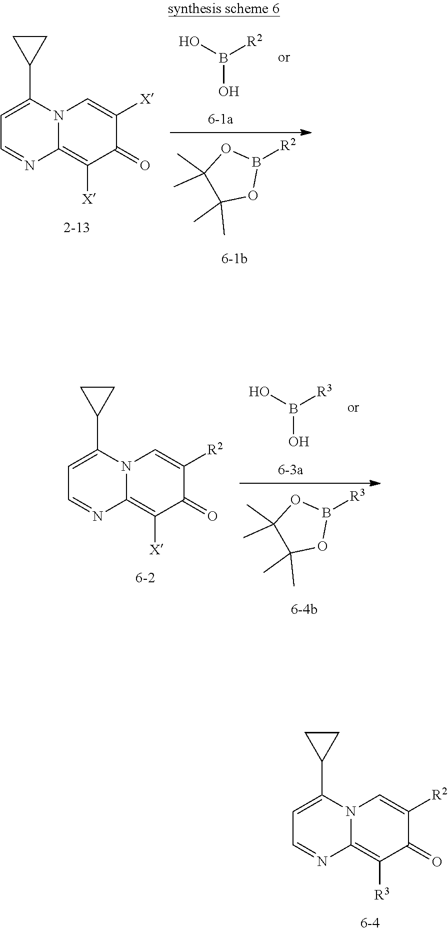
wherein X′ is selected from chlorine, bromine, or iodine; R² and R³ are defined as in the compound of formula I; and
a compound of formula 2-13 is condensed with a compound of formula 6-1a or 6-1b under alkaline conditions to obtain a compound of formula 6-2, and the compound of formula 6-2 is condensed with a compound of formula 6-3a or 6-3b under alkaline conditions to obtain a compound of formula 6-4.

11. The compound or a pharmaceutically acceptable salt thereof according to claim 2, wherein R¹, R⁴, R⁵, R⁶, and R⁷ are independently selected from hydrogen, halogen, hydroxyl, NH₂, (C1-C6 alkyl)-NR⁸—, (C1-C6 alkyl)-O—, C1-C6 alkyl, C3-C6 cycloalkyl, or 6-10-membered aryl, wherein the C1-C6 alkyl, (C1-C6 alkyl)-NR⁸—, (C1-C6 alkyl)-O—, or C3-C6 cycloalkyl is optionally substituted by halogen, cyano, hydroxyl, or —NR⁸R⁹; the 6-10-membered aryl is optionally substituted by halogen substituted C1-C3 alkoxy;
preferably, R¹, R⁴, R⁵, R⁶, and R⁷ are independently selected from hydrogen, halogen, hydroxyl, NH₂, (C1-C6 alkyl)-NR⁸—, (C1-C6 alkyl)-O—, C1-C6 alkyl, C3-C6 cycloalkyl, or 6-10-membered aryl, wherein the C1-C6 alkyl, (C1-C6 alkyl)-NR⁸—, (C1-C6 alkyl)-O—, or C3-C6 cycloalkyl is optionally substituted by halogen; the 6-10-membered aryl is optionally substituted by halogen substituted C1-C3 alkoxy;
more preferably, R¹, R⁴, R⁵, R⁶, and R⁷ are independently selected from hydrogen, halogen, hydroxyl, NH₂, (C1-C6 alkyl)-NR⁸—, (C1-C6 alkyl)-O—, C1-C6 alkyl, C3-C6 cycloalkyl, or 6-10-membered aryl, wherein the C1-C6 alkyl, (C1-C6 alkyl)-NR⁸—, (C1-C6 alkyl)-O—, or C3-C6 cycloalkyl is optionally substituted by fluorine; the 6-10-membered aryl is optionally substituted by fluorine substituted C1-C3 alkoxy;
preferably, R¹ is hydrogen; and
preferably, R⁷ is hydrogen.

12. The compound or a pharmaceutically acceptable salt thereof according to claim 2, wherein R⁴ is selected from hydrogen, C1-C6 alkyl, or C3-C6 cycloalkyl; preferably, R⁴ is selected from hydrogen, cyclopropyl, or C1-C6 alkyl; preferably, R⁴ is selected from hydrogen or C1-C6 alkyl; more preferably, R⁴ is selected from hydrogen, methyl, or cyclopropyl; further preferably, R⁴ is selected from hydrogen or methyl;
preferably, R⁵ is selected from hydrogen or C1-C6 alkyl; preferably, R⁵ is selected from hydrogen or methyl; more preferably, R⁵ is selected from hydrogen; and
preferably, R⁶ is selected from hydrogen, chlorine, hydroxyl, cyclopropyl, CF₃CH₂O—, CHF₂O—, CF₃CH₂NH—, 4-difluoromethoxylphenyl, or CH₃CH₂O—; preferably, R⁶ is selected from cyclopropyl, CF₃CH₂O—, CF₃CH₂NH—, or CH₃CH₂O—; and more preferably, R⁶ is selected from cyclopropyl, CF₃CH₂O—, or CF₃CH₂NH—.

13. The compound or a pharmaceutically acceptable salt thereof according to claim 2, wherein R² and R³ are independently selected from phenyl, naphthyl, 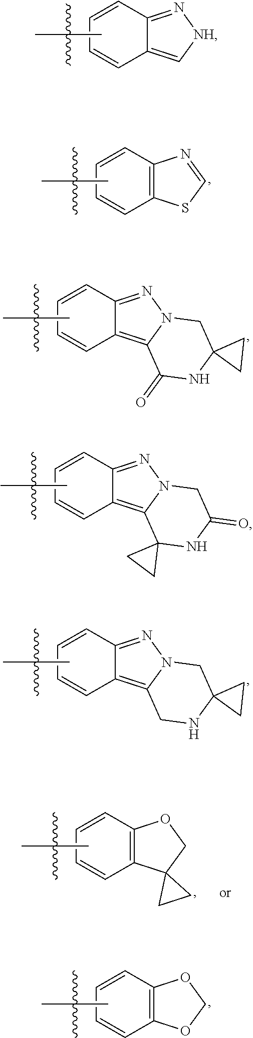
wherein the group is optionally substituted by halogen, hydroxyl, cyano, —NR⁸R⁹, NO₂, —NR¹⁰C(O)R¹¹, C1-C6 alkyl, (C1-C6 alkyl)-O—, —C(O)NR¹⁰R¹¹, or 5-7-membered heteroaryl; the C1-C6 alkyl, (C1-C6 alkyl)-O—, or 5-7-membered heteroaryl is optionally substituted by halogen, cyano, hydroxyl, C1-C3 alkyl, (C1-C3 alkyl)-O—, or —NR⁸R⁹;
preferably, R² and R³ are independently selected from phenyl, naphthyl, 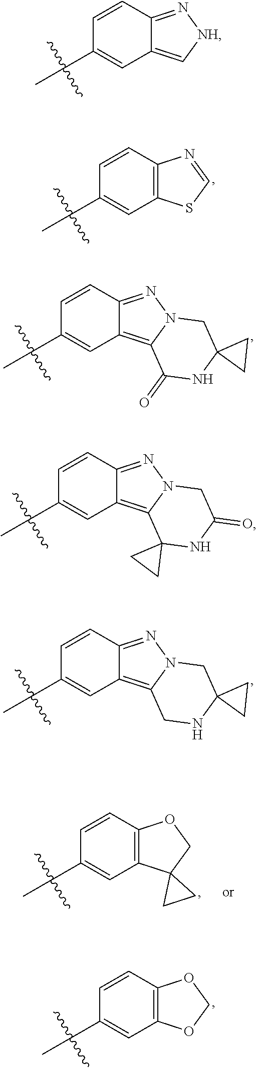
wherein the group is optionally substituted by halogen, hydroxyl, cyano, —NR⁸R⁹, NO₂, —NR¹⁰C(O)R¹¹, C1-C6 alkyl, (C1-C6 alkyl)-O—, —C(O)NR¹⁰R¹¹, or 5-7-membered heteroaryl; the C1-C6 alkyl, (C1-C6 alkyl)-O—, or 5-7-membered heteroaryl is optionally substituted by halogen, cyano, hydroxyl, C1-C3 alkyl, (C1-C3 alkyl)-O—, or —NR⁸R⁹;
more preferably, R² and R³ are independently selected from phenyl, naphthyl, 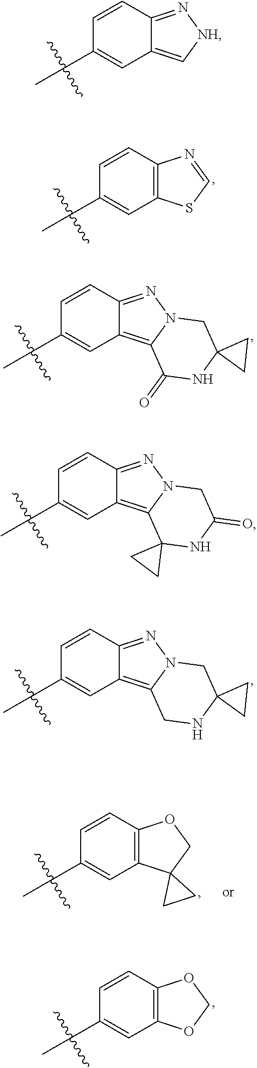
wherein the group is optionally substituted by halogen, cyano, —NR⁸R⁹, —NR¹⁰C(O)R¹¹, C1-C6 alkyl, (C1-C6 alkyl)-O—, or 5-7-membered heteroaryl; and the C1-C6 alkyl, (C1-C6 alkyl)-O—, or 5-7-membered heteroaryl is optionally substituted by methyl, halogen, or cyano;
wherein R⁸, R⁹, R¹⁰, and R¹¹ are independently selected from H or C1-C6 alkyl;
preferably, R⁸, R⁹, and R¹⁰ are independently H; and
preferably, R¹¹ is selected from C1-C6 alkyl; and preferably, R¹¹ is methyl.

14. The compound or a pharmaceutically acceptable salt thereof according to claim 2, wherein R² is selected from phenyl, 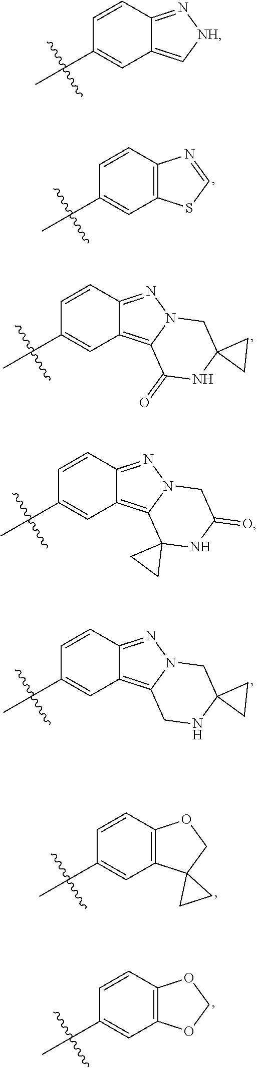
wherein the group is optionally substituted by difluoromethoxy, methyl, NH₂, methoxy, fluorine, cyanomethyl, CH₃CONH—, 1-methyl-1H-imidazol-4-yl, or 1-methyl-1H-pyrazol-5-yl; preferably, the group is optionally substituted by difluoromethoxy, methyl, NH₂, methoxy, fluorine, cyanomethyl, or 1-methyl-1H-pyrazol-5-yl;
preferably, R² is selected from 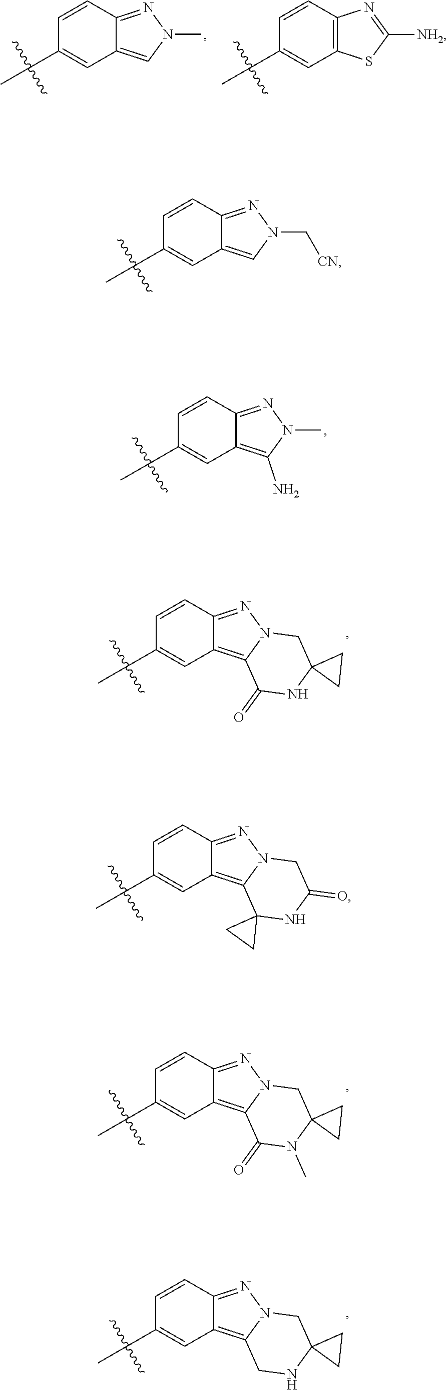 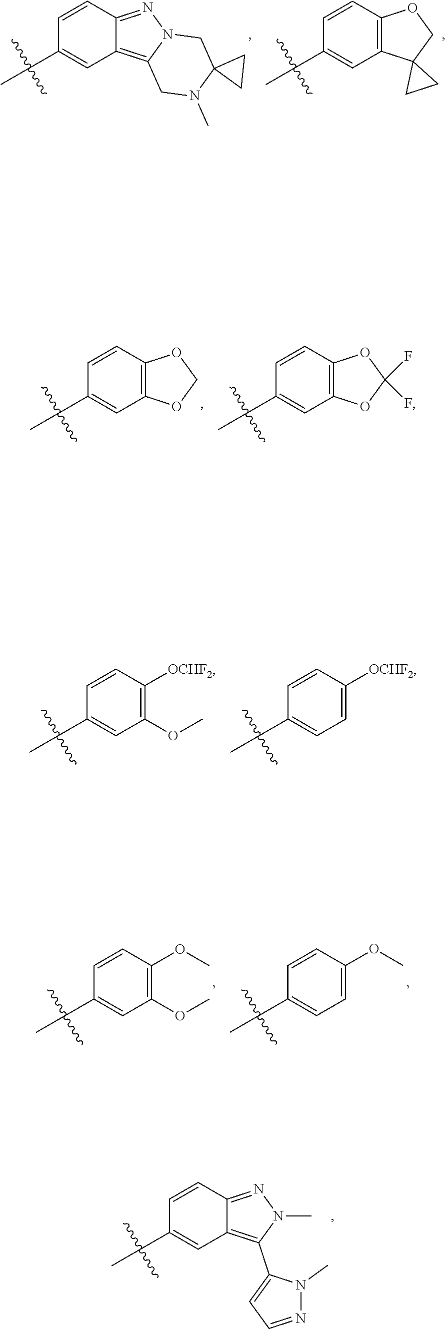 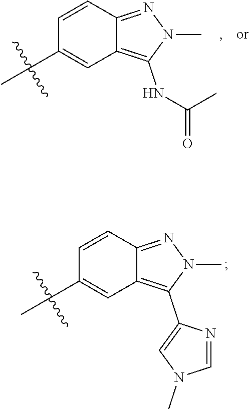 [SMILES: *c1ccc2nn(C)c(NC(C)=O)c2c1 ; *c1ccc2nn(C)c(-c3cn(C)cn3)c2c1]
preferably, R² is selected from 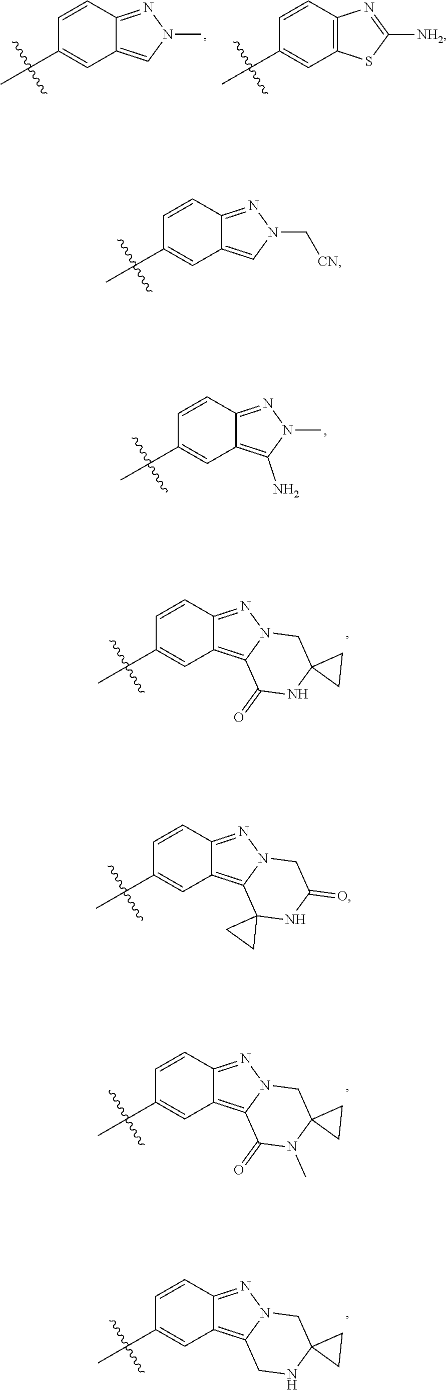 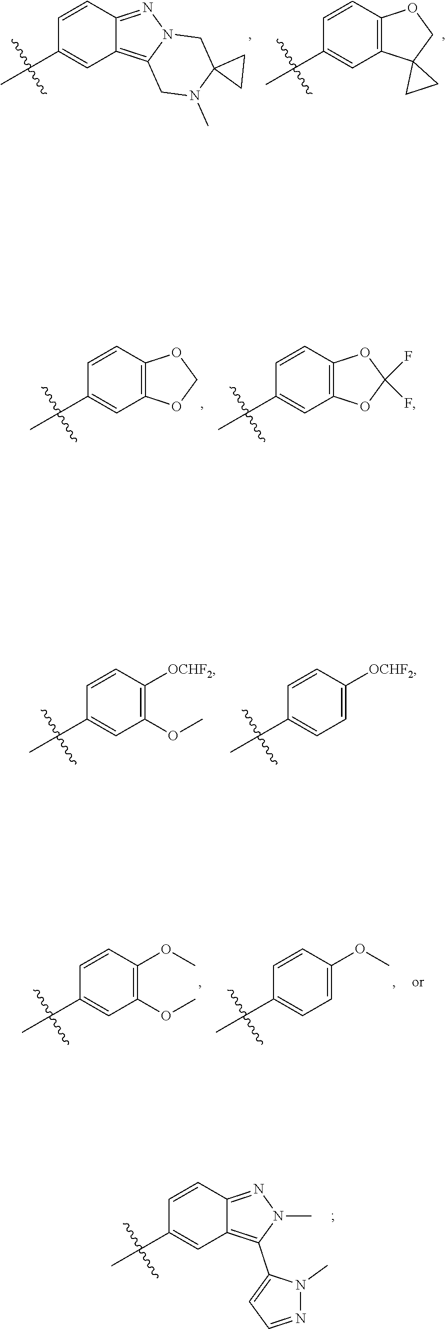
more preferably, R² is selected from 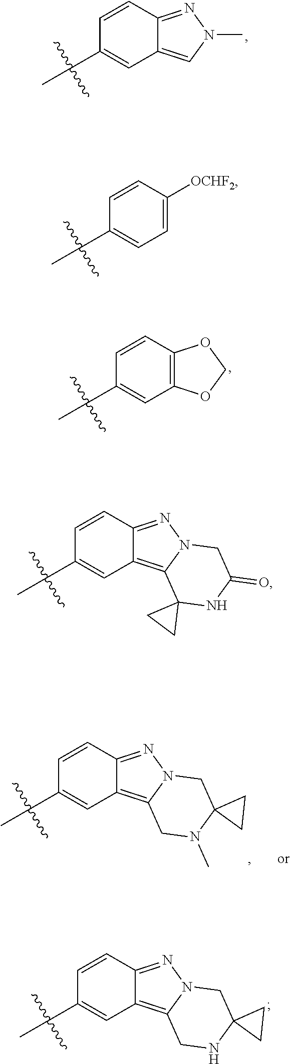 [SMILES: *c1ccc2nn(C)cc2c1 ; *c1ccc(OC(F)F)cc1 ; *c1ccc2c(c1)OCO2 ; *c1ccc2nn3c(c2c1)C1(CC1)NC(=O)C3 ; *c1ccc2nn3c(c2c1)CN(C)C1(CC1)C3 ; *c1ccc2nn3c(c2c1)CNC1(CC1)C3]
further referably R² is selected from 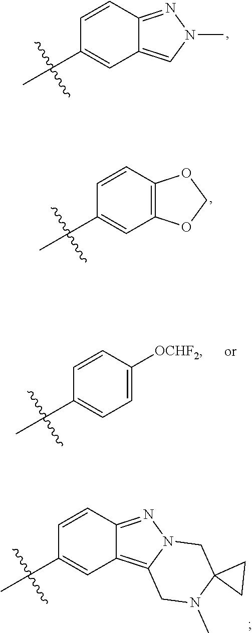 [SMILES: *c1ccc2nn(C)cc2c1 ; *c1ccc2c(c1)OCO2 ; *c1ccc(OC(F)F)cc1 ; *c1ccc2nn3c(c2c1)CN(C)C1(CC1)C3]
preferably, R³ is selected from phenyl, 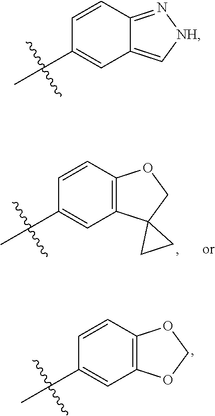 [SMILES: *c1ccc2n[nH]cc2c1 ; *c1ccc2c(c1)C1(CC1)CO2 ; *c1ccc2c(c1)OCO2]
wherein the group is optionally substituted by difluoromethoxy, methyl, NH₂, fluorine, methoxy, CH₃CONH-1-methyl-1H-imidazol-4-yl, or 1-methyl-1H-pyrazol-5-yl; preferably, the group is optionally substituted by difluoromethoxy, methyl, NH₂, fluorine, methoxy, or 1-methyl-1H-pyrazol-5-yl;
preferably, R³ is selected from 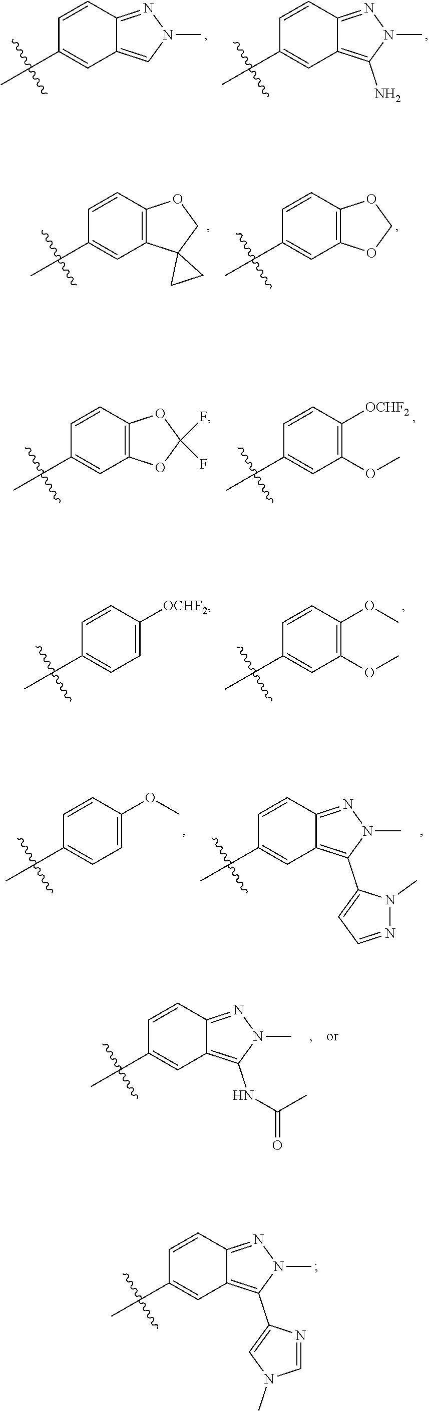
preferably, R³ is selected from 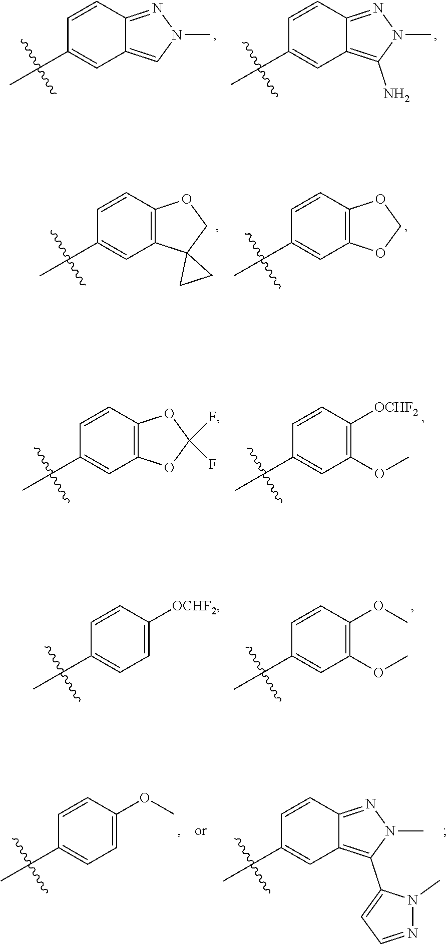
more preferably, R³ is selected from 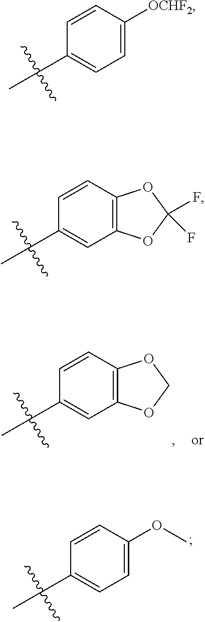
and and
further preferably, R³ is selected from 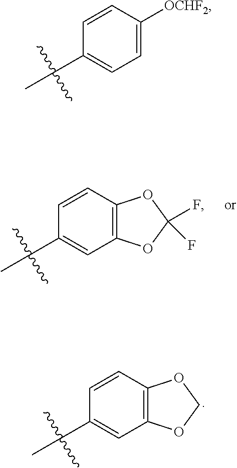 [SMILES: *c1ccc(OC(F)F)cc1 ; *c1ccc2c(c1)OC(F)(F)O2 ; *c1ccc2c(c1)OCO2]
